(19) United States
(12) Patent Application Publication
(10) Pub. No.: US 2012/0214808 A1
(43) Pub. Date: Aug. 23, 2012

(54) Aryl Urea Derivatives for Treating Obesity

(75) Inventors: Jason Bloxham, Oxford (GB); Matthew Colin Thor Fyfe, Oxford (GB); James Horswill, Oxford (GB); Revathy Perpetua Jeevaratnam, Oxford (GB); John Keily, Oxford (GB); Martin James Procter, Oxford (GB); Karen Lesley Schofield, Oxford (GB); Salam Shaaban, Westborough, MA (US); Simon Andrew Swain, Oxford (GB); Philippe Wong-Kai-In, Oxford (GB)

(73) Assignee: Prosidion Limited, Oxford (GB)

(21) Appl. No.: 13/399,517

(22) Filed: Feb. 17, 2012

Related U.S. Application Data
(63) Continuation of application No. 11/660,405, filed on May 5, 2008, now abandoned.
(60) Provisional application No. 60/602,268, filed on Aug. 16, 2004.

(51) Int. Cl. A61K 31/5377; C07D 277/38; A61K 31/381; C07D 333/50; C07D 333/36; A61K 31/17; C07C 275/42; A61K 31/4164; C07D 233/56; A61K 31/428; C07D 277/82; A61K 31/44; C07D 213/72; A61K 31/5375; C07D 295/12; A61K 31/505; C07D 239/02; C07D 413/04; A61P 3/04; A61P 25/18; A61P 25/24; A61P 3/00; A61P 25/08; A61P 25/00; A61K 31/426

(52) U.S. Cl. 514/235.5; 514/371; 548/196; 514/443; 549/57; 514/447; 549/69; 514/598; 564/52; 514/399; 548/343.5; 514/367; 548/163; 514/349; 546/297; 514/231.2; 544/166; 514/256; 544/335; 544/131

(57) ABSTRACT

A method of treating a condition associated with the CB-1 receptor, in particular obesity, by administering an effective amount of an aryl urea CB-1 receptor modulating compound to a subject in need of such treatment.

### BACKGROUND OF THE INVENTION

The present invention is directed to aryl urea derivatives. In particular, the present invention is directed to aryl urea derivatives useful in the treatment of conditions associated with the cannabinoid 1 receptor, in particular obesity.

Obesity, defined as a high ratio of body fat to lean body mass, is understood to be a risk factor for several potentially life-threatening diseases including atherosclerosis, hypertension, type II diabetes, stroke, pulmonary embolism, gallbladder disease, sleep apnea, and colon and postmenopausal breast cancer. Thus, the number of people suffering from such diseases can be lowered if obesity can be minimized without increasing other risk factors.

Presently, obesity treatments include diets to lower the caloric intake, and exercises to increase the caloric outflow. As the continuing onslaught of new diet and exercise regimes show, such programs are often ineffective because many patients have difficulty following such programs long-term. Surgery to physically remove fat or surgery, such as gastric partitioning, jejunoileal bypass, and bagotomy, to reduce stomach capacity, entail considerable risk. Thus, there remains a need for new procedures to treat obesity.

Obesity treatments also include administering drugs. As described in D. Spanswick and K. Lee, Expert Opinion, 8(1):217-237 (2003), such drugs include appetite suppressants, inhibitors of fat absorption, enhancers of energy expenditure, and stimulators of fat mobilization. Among the various central nervous system (CNS) sites susceptible as therapeutic targets for anti-obesity drugs is the cannabinoid 1 (CB1, CB-1 or CB₁) receptor. Inhibition of the CB-1 receptor by, for example, administering a CB-1 antagonist acts to suppress appetite. Further, inhibition of CB-1 is useful for the prophylactic use to prevent overweight, to assist in regulating food intake, and to assist as a diet aid. Compounds that target the CB-1 receptor include SR-141716, a selective CB-1 receptor antagonist (see ibid. at 230). Nevertheless, it would be desirable to develop other compounds that inhibit CB-1 for the treatment of obesity.

As described above, inhibition of the CB-1 receptor is useful to suppress appetite, to prophylactically prevent overweight, to assist in regulating food intake, to assist as a diet aid, and to treat obesity. Such inhibition includes modulating the CB-1 receptor by applying an antagonist or by applying an inverse agonist. Thus, there is a need for novel compounds and novel administration of CB-1 modulators, e.g. antagonist or inverse agonist compounds, to suppress appetite, to prophylactically prevent overweight, to assist in regulating food intake, to assist as a diet aid, and to treat obesity.

As the CB-1 receptor seems to be involved in the brain's reward system, CB-1 modulator compounds may also find use in the treatment of addictive disorders such as tobacco smoking, heroin addiction (see Solinas M et al, J. Pharmacol. Exp. Ther., 2003 July; 306(1):93-102); relapse to cocaine-seeking (see De Vries T J et al, Nat. Med., 2001 October; 7(10):1151-4); and alcoholism (see Hungund B L et al, J. Neurochem., 2003 February; 84(4):698-704). CB-1 is also involved in other central functions besides the rewards system. CB-1 receptor activation by cannabis or other CB-1 agonists leads to memory impairment. CB-1 antagonists are therefore good candidate agents for memory enhancement (see Reibaud M et al, Eur. J. Pharmacol., 1999 Aug. 20; 379(1):R1-2, and Terranova J P et al, Psychopharmacology (Berl)., 1996 July; 126(2):165-72). CB-1 activation can also lead to impairment in movement and movement disorders like Parkinson's disease have been associated with elevated brain endocannabinoids. CB-1 antagonism would therefore be a good candidate treatment for Parkinson's disease (see Di Marzo V et al, FASEB J., 2000 July; 14(10): 1432-8).

Central CB-1 receptor signaling is functionally linked to monoaminergic neurotransmission. This makes CB-1 antagonists candidates for the treatment of psychosis, affective and cognitive disorders brought about by disturbances in any of the central monoaminergic systems.

In addition to its strong central expression, CB-1 is expressed in some peripheral tissues. CB-1 receptors expressed on nerve endings in the gastrointestinal tract depress gastrointestinal motility, mainly by inhibiting ongoing contractile transmitter release. Antagonists of CB-1 receptor could thus find use in pathological states consisting of decreased intestinal motility such as Paralytic ileus caused by peritonitis, surgery, or other noxious situations (see Mascolo N et al, FASEB J., 2002 December; 16(14):1973-5).

CB-1 receptors also play a role in vascular endothelial cells where they mediate the hypotensive effects of platelet and macrophage-derived endocannabinoids. CB-1 antagonists would be useful agents in inhibiting endotoxin-induced or cirrhotic hypotension (see Batkai S et al, Nat. Med., 2001 July; 7(7):827-32) both of which are characterized by elevated levels of endocannabinoids.

Various aryl urea derivatives are known, however the use of such compounds as CB-1 receptor modulators has not previously been described or suggested.

### SUMMARY OF THE INVENTION

A method of treating a condition, e.g. obesity, associated with the CB-1 receptor, by administering an effective amount of an aryl urea CB-1 receptor modulator compound to a subject in need of such treatment.

### DETAILED DESCRIPTION OF THE INVENTION

The present invention provides a method of treating a condition associated with the CB-1 receptor by administering to a subject in need of such treatment a compound of formula (I):

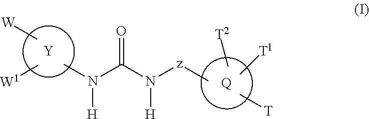

or a pharmaceutically acceptable salt thereof, wherein:

Y is phenyl, a 5- or 6-membered heteroaryl group, or a 9-membered bicyclic heteroaryl group attached to the urea through the 5-membered ring;

W is COOR¹, COR¹, C₁₋₆alkyl, C₁₋₃-fluoroalkyl, C₁₋₆alkoxy, phenoxy, C₁₋₃-fluoroalkoxy, C₁₋₃alkoxyC₁₋₃alkoxy, C₁₋₆alkylthio, C₃₋₆cycloalkyl, chloro, fluoro, nitrile, —(CH₂)ₘ—NR²R³, —O(CH₂)ₙ—NR²R³, or 5- or 6-membered heteroaryl optionally substituted by 1 or 2 groups independently selected from C₁₋₃alkyl, C₁₋₃-fluoroalkyl, C₁₋₃alkoxy, C₁₋₃-fluoroalkoxy, C₁₋₃alkoxyC₁₋₃alkyl, chloro, fluoro and —(CH₂)ₘ—NR²R³; or when Y is a 9-membered bicyclic heteroaryl group attached to the urea through the 5-membered ring, or when Z is C₁₋₃alkylene or C₂₋₃alkenylene, then W may be hydrogen;

W¹ is hydrogen, halogen, C₁₋₃alkyl, hydroxy or C₁₋₃alkoxy;

or W and W¹, when attached to adjacent carbon atoms on Y, together form a group —O—(CH₂)ₚ—O—, wherein p is 1, 2 or 3;

or the group formed from —Y, -(W) and -(W¹) is:

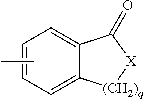

wherein X is O or CH₂ and q is 1 or 2;

Z is C₁₋₃alkylene, C₂₋₃alkenylene or a bond;

Q is phenyl, or a 5- to 10-membered mono- or bicyclic heteroaryl group;

T is hydrogen, halogen, nitro, nitrile, COOR¹, COR¹, —(CH₂)ₘ—NR²R³, CONHCH₂COOH, C₁₋₆alkyl optionally substituted by COOR⁴ or OR⁴, C₁₋₃-fluoroalkyl, C₁₋₆alkoxy, C₁₋₃-fluoroalkoxy, C₁₋₆alkylthio, SOR⁵, SO₂R⁵; or a C₃₋₆cycloalkyl group, 5- to 7-membered heterocyclyl group or 5- to 10-membered heteroaryl group any one of which is optionally substituted by 1 or 2 groups independently selected from C₁₋₃alkyl, C₁₋₃-fluoroalkyl, C₁₋₃alkoxy, C₁₋₃-fluoroalkoxy, C₁₋₃alkoxyC₁₋₃alkyl, chloro, fluoro, hydroxy and —(CH₂)ₘ—NR²R³;

T¹ and T² are independently selected from hydrogen, halogen, hydroxy, C₁₋₃alkyl and C₁₋₃alkoxy;

or T and T¹, when attached to adjacent carbon atoms on Q, together form a group —O—(CH₂)ₚ—O—, wherein p is 1, 2 or 3;

m is 0, 1, 2 or 3;

n is 2 or 3;

R¹ is C₁₋₆alkyl, C₃₋₆cycloalkyl, phenyl or a 5- or 6-membered heteroaryl or heterocyclyl group;

R² and R³ are independently selected from hydrogen, C₁₋₆alkyl and C₃₋₆cycloalkyl, or R² and R³ together with the nitrogen to which they are attached form a 5- to 7-membered heterocyclic ring optionally containing an additional heteroatom selected from O, S and NR⁴, and optionally substituted by 1 or 2 groups independently selected from C₁₋₃alkyl, fluoro and hydroxy;

R⁴ is hydrogen or C₁₋₃alkyl; and

R⁵ is C₃₋₆alkyl or C₃₋₆cycloalkyl.

The invention also provides the use of a compound of formula (I), or a pharmaceutically acceptable salt thereof, in the treatment of a condition associated with the CB-1 receptor.

The invention also provides the use of a compound of formula (I), or a pharmaceutically acceptable salt thereof, in the manufacture of a medicament for the treatment of a condition associated with the CB-1 receptor.

The molecular weight of the compounds of formula (I) is preferably less than 800, more preferably less than 600.

Particular examples of 5- or 6-membered heteroaryl groups that Y may represent include thienyl, thiazolyl and thiadiazolyl.

Particular examples of 9-membered bicyclic heteroaryl groups that Y may represent include benzothienyl and benzothiazolyl, especially benzothien-2-yl and benzothiazol-2-yl.

A specific group of compounds of formula (I) which may be mentioned are those where Y is phenyl.

When Y is phenyl, W is preferably COOR¹ especially COOEt, or COR¹, C₁₋₆alkoxy, C₁₋₆alkylthio, fluoro, chloro, C₁₋₃alkoxyC₁₋₃alkoxy, —(CH₂)ₘ—NR²R³, —O(CH₂)ₙ—NR²R³, or 5- or 6-membered heteroaryl optionally substituted by C₁₋₃alkyl. Particular W groups which may be mentioned are chloro, C₁₋₃alkoxyC₁₋₃alkoxy, —(CH₂)ₘ—NR²R³ and —O(CH₂)ₙ—NR²R³ where —NR²R³, is preferably morpholinyl.

Heteroaryl groups which W may represent include 5- or 6-membered heteroaryl groups containing 1 or 2 nitrogen atoms such as pyrazole, pyrrole, imidazole, pyrimidine or pyridine.

W¹ is preferably hydrogen, halogen or C₁₋₃alkoxy, more preferably hydrogen.

Z is preferably C₂alkylene, C₂alkenylene or a bond, more preferably C₂alkylene or a bond, especially a bond.

Q is preferably phenyl, pyridyl or a 9-membered bicyclic heteroaryl group such as benzothienyl, benzothiazolyl, or indazole, especially benzothien-2-yl, benzothiazol-2-yl, or indazol-5-yl.

Q is more preferably phenyl, or a 9-membered bicyclic heteroaryl group such as benzothienyl or benzothiazolyl, especially benzothien-2-yl or benzothiazol-2-yl.

A specific group of compounds of formula (I) which may be mentioned are those where Q is phenyl or pyridyl, especially phenyl.

A group of compounds which may be mentioned are those where T is hydrogen, halogen, nitro, nitrile, COOR¹, COR¹, —(CH₂)ₘ—NR²R³, CONHCH₂COOH, C₁₋₆alkyl optionally substituted by COOR⁴ or OR⁴, C₁₋₃-fluoroalkyl, C₁₋₆alkoxy, C₁₋₃-fluoroalkoxy, C₁₋₆alkylthio, SOR⁵, SO₂R⁵; or a C₃₋₆cycloalkyl group, or a 5- or 6-membered heterocyclyl or heteroaryl group any one of which is optionally substituted by 1 or 2 groups independently selected from C₁₋₃alkyl, C₁₋₃-fluoroalkyl, C₁₋₃alkoxy, C₁₋₃-fluoroalkoxy, C₁₋₃alkoxyC₁₋₃alkyl, chloro, fluoro and —(CH₂)ₘ—NR²R³, wherein m is 0, 1, 2 or 3.

T is preferably halogen, COOR¹, COR¹, —(CH₂)ₘ—NR²R³ optionally substituted by 1 or 2 groups independently selected from C₁₋₃alkyl, fluoro and hydroxy, or a 5- to 10-membered heteroaryl group optionally substituted by C₁₋₃alkyl, e.g. a 5- or 6-membered heteroaryl group containing 1 or 2 nitrogen atoms such as pyrazole, pyrrole, imidazole, pyrimidine or pyridine, or thiazole, thiadiazole, oxazole or 3,4-dihydro-1H-isoquinolin-2-yl.

T¹ and T² are preferably hydrogen, halogen or hydroxy, more preferably hydrogen or halogen.

T² is preferably hydrogen.

A specific group of compounds which may be mentioned are those where T is —(CH₂)ₘ—NR²R³, T′ is halogen, e.g. fluoro, and T² is hydrogen.

When T is —(CH₂)ₘ—NR²R³, m is preferably 0 and R² and R³ together with the nitrogen to which they are attached preferably form a 5- to 7-membered heterocyclic ring, e.g. a piperidine ring, optionally substituted by 1 or 2 groups independently selected from C₁₋₃alkyl, fluoro and hydroxy, e.g. methyl.

W and T are preferably different.

Preferably at least one of Y and Q is phenyl.

Substituents on the groups Y and Q are preferably in the meta and/or para positions relative to the urea, more preferably the para position.

A group of compounds which may be mentioned are those where R¹ is C₁₋₆alkyl, C₃₋₆cycloalkyl, phenyl or a 5- or 6-membered heteroaryl group.

While the preferred groups for each variable have generally been listed above separately for each variable, preferred compounds of this invention include those in which several or each variable in formula (I) is selected from the preferred, more preferred or particularly listed groups for each variable. Therefore, this invention is intended to include all combinations of preferred, more preferred and particularly listed groups. The preferences listed above also apply, where applicable, to the compounds of formula (Ia) below.

Specific compounds which may be used in the method of the invention include:
2-[3-(4-Fluorophenyl)ureido]-4-methylthiazole-5-carboxylic acid ethyl ester
2-[3-(3-Fluorophenyl)ureido]-4-methylthiazole-5-carboxylic acid ethyl ester
2-[3-(4-Ethoxycarbonylphenyl)ureido]-4-methylthiazole-5-carboxylic acid ethyl ester
4-Methyl-2-[3-(4-methylsulfanylphenyl)ureido]thiazole-5-carboxylic acid ethyl ester
4-Methyl-2-[3-phenylureido]thiazole-5-carboxylic acid ethyl ester
1-(4-Acetylphenyl)-3-benzo[b]thiophen-2-ylurea
1-Benzo[b]thiophen-2-yl-3-(4-methanesulfonylphenyl)urea
1-Benzo[b]thiophen-2-yl-3-(2-methylphenyl)urea
1-Benzo[b]thiophen-2-yl-3-(3,4-dihydro-2H-benzo[b][1,4]dioxepin-7-yl)urea
1-Benzo[b]thiophen-2-yl-3-phenylurea
1-Benzo[b]thiophen-2-yl-3-(2,4-difluorophenyl)urea
1-Benzo[b]thiophen-2-yl-3-(4-fluorophenyl)urea
1-(4-Fluorophenyl)-3-(4-methylthiophen-2-yl)urea
1-Phenyl-3-(2-thiophen-2-ylvinyl)urea
1-(2-Chlorophenyl)-3-(2-thiophen-2-ylvinyl)urea
4-[3-(4-Fluoro-2-methylphenyl)ureido]benzoic acid ethyl ester
4-[3-(2,4,6-Trifluorophenyl)ureido]benzoic acid ethyl ester
4-[3-(2,4-Difluorophenyl)ureido]benzoic acid ethyl ester
4-[3-(3,4-Difluorophenyl)ureido]benzoic acid ethyl ester
4-[3-(2-Chloro-4-fluorophenyl)ureido]benzoic acid ethyl ester
4-[3-(4-Fluoro-3-methylphenyl)ureido]benzoic acid ethyl ester
4-[3-(3-Chloro-4-fluorophenyl)ureido]benzoic acid ethyl ester
4-[3-(4-Fluoro-3-methoxyphenyl)ureido]benzoic acid ethyl ester
4-[3-(3-Fluorophenyl)ureido]benzoic acid ethyl ester
4-[3-(2-Fluorophenyl)ureido]benzoic acid ethyl ester
1-(4-Ethoxyphenyl)-3-(4-fluorophenyl)urea
4-[3-(4-Fluorophenyl)ureido]-3-methylbenzoic acid methyl ester
4-[3-(4-Fluorophenyl)ureido]-3-hydroxybenzoic acid methyl ester
1-(3-Ethoxyphenyl)-3-(4-fluorophenyl)urea
1-(4-Fluorophenyl)-3-(4-methoxyphenyl)urea
1-(4-Cyanophenyl)-3-(4-fluorophenyl)urea
1-(4-Acetylphenyl)-3-(4-fluorophenyl)urea
4-[3-(4-Fluoro-3-nitrophenyl)ureido]benzoic acid ethyl ester
4-[3-(4-Fluorophenyl)ureido]benzoic acid methyl ester
1-(4-Chlorophenyl)-3-(4-ethoxyphenyl)urea
1,3-Bis(4-acetylphenyl)urea
1-(4-Acetylphenyl)-3-(3-chlorophenyl)urea
1-(4-Acetylphenyl)-3-(4-chlorophenyl)urea
4-(3-Phenylureido)benzoic acid ethyl ester
1-(2-Thiophen-2-ylethyl)-3-(4-methylphenyl)urea
1-(4-Methoxyphenyl)-3-(2-thiophen-2-ylethyl)urea
1-(2-Thiophen-2-ylethyl)-3-(4-trifluoromethoxyphenyl)urea
1-(4-Difluoromethoxyphenyl)-3-(2-thiophen-2-ylethyl)urea
1-(4-Ethylphenyl)-3-(2-thiophen-2-ylethyl)urea
1-(2-Thiophen-2-ylethyl)-3-(4-trifluoromethylphenyl)urea
1-(3-Chlorophenyl)-3-(2-thiophen-2-ylethyl)urea
1-(4-Butylphenyl)-3-(2-thiophen-2-ylethyl)urea
1-(4-Acetylphenyl)-3-(2-thiophen-2-ylethyl)urea
1-(3-Ethylphenyl)-3-(2-thiophen-2-ylethyl)urea
1-(4-Fluorophenyl)-3-(2-thiophen-2-ylethyl)urea
1-(4-Chlorophenyl)-3-(2-thiophen-2-ylethyl)urea
1-Phenyl-3-(2-thiophen-2-ylethyl)urea
1-(4-Methylsulfanylphenyl)-3-(2-thiophen-2-ylethyl)urea
1-(3-Chloro-4-fluorophenyl)-3-(2-thiophen-2-ylethyl)urea
1-(4-Isopropylphenyl)-3-(2-thiophen-2-ylethyl)urea
4-(3-Benzothiazol-6-ylureido)benzoic acid ethyl ester
4-[3-(4-Imidazol-1-ylphenyl)ureido]benzoic acid ethyl ester
4-[3-(6-Fluorobenzothiazol-2-yl)ureido]benzoic acid ethyl ester
5-[3-(4-Ethoxycarbonylphenyl)ureido]furan-2-carboxylic acid methyl ester
4-[3-(1H-Indol-6-yl)ureido]benzoic acid ethyl ester
4-[3-(3-Methoxycarbonylphenyl)ureido]benzoic acid ethyl ester
2-[3-(4-Ethoxycarbonylphenyl)ureido]thiophene-3-carboxylic acid methyl ester
4-{3-[2-(1-Methyl-1H-pyrrol-2-yl)ethyl]ureido}benzoic acid ethyl ester
4-[3-(6-Methoxypyridin-3-yl)ureido]benzoic acid ethyl ester
4-[3-(4-Ethoxycarbonylphenyl)ureido]nicotinic acid methyl ester
4-[3-(6-Chloropyridin-3-yl)ureido]benzoic acid ethyl ester
4-[3-(4-Carboxymethylphenyl)ureido]benzoic acid ethyl ester
4-[3-(1H-Indol-5-yl)ureido]benzoic acid ethyl ester
4-(3-Benzothiazol-2-ylureido)benzoic acid ethyl ester
4-(3-[1,3,4]Thiadiazol-2-ylureido)benzoic acid ethyl ester
4-[3-(4-Fluorophenyl)ureido]benzoic acid ethyl ester
1-(4-Fluorophenyl)-3-(4-morpholin-4-ylphenyl)urea
1-Benzothiazol-6-yl-3-(4-fluorophenyl)urea
1-(4-Fluorophenyl)-3-(4-imidazol-1-ylphenyl)urea
6-[3-(4-Fluorophenyl)ureido]nicotinic acid methyl ester
1-(6-Chlorobenzothiazol-2-yl)-3-(4-fluorophenyl)urea
1-(6-Fluorobenzothiazol-2-yl)-3-(4-fluorophenyl)urea
1-(4,6-Difluorobenzothiazol-2-yl)-3-(4-fluorophenyl)urea
1-(4-Fluorophenyl)-3-(6-methoxybenzothiazol-2-yl)urea
1-(4-Fluorophenyl)-3-[3-(2-methylpyrimidin-4-yl)phenyl]urea
3-[3-(4-Fluorophenyl)ureido]benzoic acid methyl ester
1-(4-Fluorophenyl)-3-(2-fluorophenyl)urea
3-[3-(4-Fluorophenyl)ureido]benzoic acid ethyl ester
1-(4-Fluoro-3-methylphenyl)-3-(4-fluorophenyl)urea
1-(4-Fluorophenyl)-3-pyridin-4-ylurea
1-Benzothiazol-2-yl-3-(4-fluorophenyl)urea
4-{3-[3-(2-Methylpyrimidin-4-yl)phenyl]ureido}benzoic acid ethyl ester
4-[3-(1-Oxoindan-5-yl)ureido]benzoic acid ethyl ester
4-[3-(6-Morpholin-4-yl-pyridin-3-yl)ureido]benzoic acid ethyl ester
2-[3-(4-Ethoxycarbonylphenyl)ureido]thiazole-5-carboxylic acid methyl ester
4-[3-(3-Ethoxycarbonylphenyl)ureido]benzoic acid ethyl ester
4-[3-(4-Ethoxycarbonylphenyl)ureido]benzoic acid propyl ester
4-[3-(4-Ethoxycarbonylphenyl)ureido]benzoic acid pentyl ester
4-[3-(4-Ethoxycarbonylphenyl)ureido]benzoic acid isobutyl ester
4-[3-(4-Ethoxycarbonylphenyl)ureido]benzoic acid phenyl ester
4-{3-[4-(1,1,2,2-Tetrafluoroethoxy)phenyl]ureido}benzoic acid ethyl ester
4-[3-(3-Oxazol-5-ylphenyl)ureido]benzoic acid ethyl ester
4-[3-(4-Ethoxycarbonylmethylphenyl)ureido]benzoic acid ethyl ester
4-[3-(4-[1,2,3]Thiadiazol-4-ylphenyl)ureido]benzoic acid ethyl ester
4-[3-(4-Propionylphenyl)ureido]benzoic acid ethyl ester
4-[3-(4-Acetylphenyl)ureido]benzoic acid ethyl ester
4-[3-(4-Benzoylphenyl)ureido]benzoic acid ethyl ester
4-{3-[4-(4,5-Dihydrooxazol-2-yl)phenyl]ureido}benzoic acid ethyl ester
4-{3-[4-(2-Methylpyrimidin-4-yl)phenyl]ureido}benzoic acid ethyl ester
1-(4-Fluorophenyl)-3-(4-pyrrol-1-ylphenyl)urea
1-(4-Fluorophenyl)-3-(2-methylbenzothiazol-5-yl)urea
1-(4-Fluorophenyl)-3-(3-oxazol-5-ylphenyl)urea
1-(4-Fluorophenyl)-3-(4-propionylphenyl)urea
1-(4-Fluorophenyl)-3-[4-(2-methylpyrimidin-4-yl)phenyl]urea
4-[3-(4-Ethoxycarbonylphenyl)ureido]benzoic acid butyl ester
2-[3-(4-Ethoxycarbonylphenyl)ureido]-4-methylpyrimidine-5-carboxylic acid ethyl ester
4-[3-(4-Oxazol-5-ylphenyl)ureido]benzoic acid ethyl ester
2-Chloro-4-[3-(4-ethoxycarbonylphenyl)ureido]benzoic acid ethyl ester
4-[3-(4-Ethoxycarbonylphenyl)ureido]-2-methoxybenzoic acid ethyl ester
4-[3-(4-Ethoxycarbonylphenyl)ureido]-3-methoxybenzoic acid ethyl ester
6-[3-(4-Ethoxycarbonylphenyl)ureido]nicotinic acid ethyl ester
4-[3-(4-Fluorophenyl)ureido]-3-hydroxybenzoic acid ethyl ester
4-[3-(3-Acetylphenyl)ureido]benzoic acid ethyl ester
4-[3-(4-Butyrylphenyl)ureido]benzoic acid ethyl ester
4-{3-[4-(1H-Pyrazol-3-yl)phenyl]ureido}benzoic acid ethyl ester
4-[3-(4-Fluorophenyl)ureido]benzoic acid propyl ester
4-[3-(4-Fluorophenyl)ureido]benzoic acid pentyl ester
4-[3-(4-Fluorophenyl)ureido]benzoic acid isobutyl ester
4-[3-(4-Fluorophenyl)ureido]benzoic acid phenyl ester
{4-[3-(4-Fluorophenyl)ureido]phenyl}acetic acid ethyl ester
1-(4-Benzoylphenyl)-3-(4-fluorophenyl)urea
1-(4-Butyrylphenyl)-3-(4-fluorophenyl)urea
4-[3-(4-Fluorophenyl)ureido]benzoic acid butyl ester
2-Chloro-4-[3-(4-fluorophenyl)ureido]benzoic acid ethyl ester
1-(4-Chlorophenyl)-3-(4-trifluoromethylphenyl)urea
1-(4-Chlorophenyl)-3-(4-cyanophenyl)urea
1-(4-Bromo-3-chlorophenyl)-3-(4-chlorophenyl)urea
4-[3-(2-Chlorophenyl)ureido]benzoic acid ethyl ester
4-[3-(4-Methylsulfanylphenyl)ureido]benzoic acid ethyl ester
4-[3-(4-Chlorophenyl)ureido]benzoic acid ethyl ester
1-(4-Chlorophenyl)-3-(4-dimethylaminophenyl)urea
1-Phenyl-3-(4-ethoxyphenyl)urea
1-(3-Chlorophenyl)-3-(4-ethoxyphenyl)urea
4-[3-(3-Chlorophenyl)ureido]benzoic acid ethyl ester
4-(3-Phenylureido)benzoic acid methyl ester
1-(3-Methylsulfanyl[1,2,4]thiadiazol-5-yl)-3-phenylurea
1-(3-Ethylsulfanyl[1,2,4]thiadiazol-5-yl)-3-phenylurea
1-(4-Chlorophenyl)-3-(2,3-dihydrobenzo[1,4]dioxan-6-yl)urea
1-(4-Acetylphenyl)-3-(3,4-dichlorophenyl)urea
1-Thiazol-2-yl-3-(4-methylphenyl)urea
5-[3-(4-Chlorophenyl)ureido]-3-methyl thiophene-2-carboxylic acid ethyl ester
{4-[3-(4-Methylsulfanylphenyl)ureido]benzoylamino}acetic acid
1-[5-(2-Methyl-5-trifluoromethyl-2H-pyrazol-3-yl)thiophen-2-yl]-3-(3-trifluoro methylphenyl)urea
1-(3,4-Dichlorophenyl)-3-(3-hydroxyphenyl)urea
1-[3-(2-Methylpyrimidin-4-yl)phenyl]-3-phenylurea
1-(3-Acetylphenyl)-3-phenylurea
1-(3-Chlorophenyl)-3-(4-methylthiazol-2-yl)urea
1-[2-(4-Fluorophenyl)ethyl]-3-(4-isopropyl phenyl)urea
1-(4-Chlorophenyl)-3-(4-trifluoromethoxyphenyl)urea
1-(4-Chlorophenyl)-3-(4-methanesulfonylphenyl)urea
1-[2-(3-Fluorophenyl)ethyl]-3-(4-isopropylphenyl)urea
1-[2-(2-Fluorophenyl)ethyl]-3-(4-isopropylphenyl)urea
1-[2-(3-Fluorophenyl)ethyl]-3-(4-trifluoromethylphenyl)urea
1-(4-Isopropylphenyl)-3-thiazol-2-ylurea
1-(4-Acetylphenyl)-3-(4-bromophenyl)urea
1-(4-Butoxyphenyl)-3-(4-chlorophenyl)urea
1-[2-(4-Chlorophenyl)ethyl]-3-[3-(2-methylpyrimidin-4-yl)phenyl]urea
1-(4-Chlorophenyl)-3-(3-pyrrol-1-ylphenyl)urea
1-(4-Chlorophenyl)-3-(4-pyrrol-1-ylphenyl)urea
1-(4-Chlorophenyl)-3-[3-(2-methylpyrimidin-4-yl)phenyl]urea
1-(4-Chlorophenyl)-3-[4-(3,4-dihydro-1H-isoquinolin-2-yl)-3-fluorophenyl]urea
1-(3,4-Dichlorophenyl)-3-(3-fluoro-4-piperidin-1-ylphenyl)urea
1-(4-Chlorophenyl)-3-[3-(6-pyrrolidin-1-ylpyridin-2-yl)phenyl]urea
1-(4-Azepan-1-yl-3-fluorophenyl)-3-(4-chlorophenyl)urea
1-(4-Chlorophenyl)-3-(3-fluoro-4-pyrrolidin-1-ylphenyl)urea
1-(3-Fluoro-4-piperidin-1-ylphenyl)-3-[4-(2-morpholin-4-ylethoxy)phenyl]urea
1-(3-Fluoro-4-piperidin-1-ylphenyl)-3-[4-(2-methoxyethoxy)phenyl]urea
1-(4-Chlorophenyl)-3-[3-(2-isopropylpyrimidin-4-yl)phenyl]urea
1-(4-Chlorophenyl)-3-(3-fluoro-4-[1,4]oxazepan-4-ylphenyl)urea
1-(3-Fluoro-4-piperidin-1-ylphenyl)-3-[4-(morpholine-4-carbonyl)phenyl]urea
1-(3-Fluoro-4-piperidin-1-ylphenyl)-3-(4-pyrrol-1-ylphenyl)urea
4-[3-(3-Fluoro-4-piperidin-1-ylphenyl)ureido]benzoic acid ethyl ester
1-(3-Fluoro-4-piperidin-1-ylphenyl)-3-[3-(2-methoxyethoxy)phenyl]urea
1-(3-Fluoro-4-piperidin-1-ylphenyl)-3-[3-(2-morpholin-4-ylethoxy)phenyl]urea
1-(4-Chlorophenyl)-3-(4-pyridin-3-ylphenyl)urea
1-(4-Chlorophenyl)-3-[3-(6-methylpyrimidin-4-yl)phenyl]urea
1-(4-Chlorophenyl)-3-[3-fluoro-4-(3-hydroxypiperidin-1-yl)phenyl]urea
1-(4-Chlorophenyl)-3-(4-pyridin-2-yl-phenyl)urea
1-(4-Chlorophenyl)-3-(4-pyridin-4-ylphenyl)urea
1-(4-Chlorophenyl)-3-[3-(2-piperidin-1-ylpyrimidin-4-yl)phenyl]urea
1-(3-Fluoro-4-piperidin-1-ylphenyl)-3-[4-(2-morpholin-4-ylethyl)phenyl]urea
1-(3-Fluoro-4-piperidin-1-ylphenyl)-3-(4-morpholin-4-ylmethylphenyl)urea
1-(2,3-Dihydrobenzofuran-6-yl)-3-(3-fluoro-4-piperidin-1-ylphenyl)urea
1-(3,5-Dimethoxyphenyl)-3-(3-fluoro-4-piperidin-1-ylphenyl)urea
1-(4-Chlorophenyl)-3-(3-pyrazol-1-ylphenyl)urea
1-(4-Chlorophenyl)-3-[3-(3,4,5,6-tetrahydro-2H-[1,2′]bipyridinyl-6′-yl)phenyl]urea
1-(4-Morpholin-4-ylmethylphenyl)-3-(4-pyrrol-1-ylphenyl)urea
1-(4-Morpholin-4-ylmethylphenyl)-3-(3-pyrrol-1-ylphenyl)urea
1-(4-Chlorophenyl)-3-[4-(4,4-difluoropiperidin-1-yl)-3-fluorophenyl]urea
1-(4-Butyrylphenyl)-3-[4-(morpholine-4-carbonyl)phenyl]urea
1-(1-Methyl-1H-indazol-5-yl)-3-(4-morpholin-4-ylmethylphenyl)urea
1-(4-Morpholin-4-ylmethylphenyl)-3-(4-pyrazol-1-ylphenyl)urea
1-(2,3-Dihydrobenzo[1,4]dioxin-6-yl)-3-(3-fluoro-4-piperidin-1-ylphenyl)urea
1-(3,5-Dichlorophenyl)-3-(3-fluoro-4-piperidin-1-ylphenyl)urea
1-(3-Chloro-4-fluorophenyl)-3-(3-fluoro-4-piperidin-1-ylphenyl)urea
1-(4-Ethylphenyl)-3-(3-fluoro-4-piperidin-1-ylphenyl)urea
1-[2-(4-Chlorophenyl)ethyl]-3-[3-(2-methyl-2H-pyrazol-3-yl)phenyl]urea
1-(4-Chlorophenyl)-3-[3-fluoro-4-(4-hydroxypiperidin-1-yl)phenyl]urea
1-(4-Chlorophenyl)-3-[3-fluoro-4-(3-methylpiperidin-1-yl)phenyl]urea
1-Benzo[1,3]dioxol-5-yl-3-(3-fluoro-4-piperidin-1-ylphenyl)urea
1-(4-Chlorophenyl)-3-[3-fluoro-4-(2-methylpiperidin-1-yl)phenyl]urea
1-(3-Fluoro-4-piperidin-1-ylphenyl)-3-(4-methoxyphenyl)urea
1-(4-Chloro-2-hydroxyphenyl)-3-(3-fluoro-4-piperidin-1-ylphenyl)urea
1-(3-Fluoro-4-piperidin-1-ylphenyl)-3-[3-(2-methylpyrimidin-4-yl)phenyl]urea
1-(4-Chlorophenyl)-3-[3-fluoro-4-(4-trifluoromethylpiperidin-1-yl)phenyl]urea
1-(4-Chlorophenyl)-3-[3-fluoro-4-(4-methylpiperidin-1-yl)phenyl]urea
1-(3-Fluoro-4-piperidin-1-ylphenyl)-3-(4-phenoxyphenyl)urea
1-(3-Fluoro-4-piperidin-1-ylphenyl)-3-(3-phenoxyphenyl)urea
1-(4-Fluorophenyl)-3-(3-fluoro-4-piperidin-1-ylphenyl)urea
1-(3-Fluoro-4-piperidin-1-ylphenyl)-3-(3-methoxyphenyl)urea
1-(4-Cyanophenyl)-3-(3-fluoro-4-piperidin-1-ylphenyl)urea
1-(4-Chlorophenyl)-3-(4-morpholin-4-ylmethylphenyl)urea
1-(4-Chloro-3-trifluoromethylphenyl)-3-(3-fluoro-4-piperidin-1-ylphenyl)urea
1-(3-Fluoro-4-piperidin-1-ylphenyl)-3-(4-trifluoromethylphenyl)urea
1-(3-Chlorophenyl)-3-(3-fluoro-4-piperidin-1-ylphenyl)urea
1-(4-Chlorophenyl)-3-(3,4,5,6-tetrahydro-2H-[1,2′]bipyridinyl-5′-yl)urea
1-(4-Chlorophenyl)-3-[4-(morpholine-4-carbonyl)phenyl]urea
1-(4-Chlorophenyl)-3-(3-dimethylaminophenyl)urea
1-(4-Chlorophenyl)-3-(3-fluoro-4-morpholin-4-ylphenyl)urea
1-[2-(4-Chlorophenyl)ethyl]-3-(3-pyrrol-1-ylphenyl)urea
1-(3,5-Dichlorophenyl)-3-(3,4,5,6-tetrahydro-2H-[1,2]bipyridinyl-5′-yl)urea
1-(3-Chlorophenyl)-3-(3,4,5,6-tetrahydro-2H-[1,2]bipyridinyl-5′-yl)urea
1-(3,5-Bis-trifluoromethylphenyl)-3-(3,4,5,6-tetrahydro-2H-[1,2′]bipyridinyl-5′-yl)urea
1-(4-Acetylphenyl)-3-[4-(morpholine-4-carbonyl)phenyl]urea
1-(4-Acetylphenyl)-3-[3-(6-methoxypyridin-2-yl)phenyl]urea
1-(4-Acetylphenyl)-3-(4-morpholin-4-ylmethylphenyl)urea
1-(4-Chlorophenyl)-3-(3-fluoro-4-piperidin-1-ylphenyl)urea
1-(3-Chloro-4-morpholin-4-ylphenyl)-3-(4-chlorophenyl)urea
1-(4-Chlorophenyl)-3-(4-piperidin-1-ylphenyl)urea
1-(4-Acetylphenyl)-3-(3,4,5,6-tetrahydro-2H-[1,2′]bipyridinyl-5′-yl)urea
1-(4-Butyrylphenyl)-3-(4-piperidin-1-ylphenyl)urea
1-[2-(4-Chlorophenyl)ethyl]-3-(4-morpholin-4-ylmethylphenyl)urea
1-(4-Chlorophenyl)-3-(1-methyl-1H-indazol-5-yl)urea
1-(3-Acetylphenyl)-3-[2-(4-chlorophenyl)ethyl]urea
1-(4-Chlorophenyl)-3-[3-(2-pyrrolidin-1-ylpyrimidin-4-yl)phenyl]urea
1-(4-Chlorophenyl)-3-(4-pyrazol-1-ylphenyl)urea
1-[2-(4-Chlorophenyl)ethyl]-3-[4-(morpholine-4-carbonyl)phenyl]urea

and pharmaceutically acceptable salts thereof.

Conditions to be treated according to the method of the invention include obesity; psychiatric disorders such as psychotic disorders, schizophrenia, bipolar disorders, depression, cognitive disorders, memory disorders, obsessive compulsive disorders, anorexia, bulimia, attention disorders, epilepsy and related conditions affective and cognitive disorders brought about by disturbances in any of the central monoaminergic systems; and neurological disorders such as Raynaud's syndrome, movement impairment, Parkinson's disease, Huntington's chorea and Alzheimer's disease. Further conditions which may be treated according to the method of the invention include immune, cardiovascular, reproductive and endocrine disorders, endotoxin-induced or cirrhotic hypotension, septic shock, diseases related to the respiratory and gastrointestinal systems such as decreased intestinal motility such as Paralytic ileus caused by peritonitis, surgery, or other noxious situations, extended abuse, addiction and relapse indications such as tobacco smoking, heroin addiction, relapse to cocaine-seeking, and alcoholism.

The condition to be treated according to the methods of the invention is preferably obesity.

In the methods of the invention the term “treatment” includes both therapeutic and prophylactic treatment.

CB-1 receptor modulator compounds for use in the methods of the invention include CB-1 antagonists.

Certain compounds of formula (I) are novel.

The present invention also provides a compound of formula (Ia):

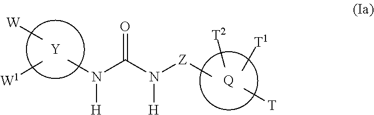

or a pharmaceutically acceptable salt thereof, wherein:

Y is phenyl, a 5- or 6-membered heteroaryl group, or a 9-membered bicyclic heteroaryl group attached to the urea through the 5-membered ring;

W is COOR¹, COR¹, C₁₋₆alkoxy, C₁₋₃-fluoroalkoxy, C₁₋₃alkoxyC₁₋₃alkoxy, —(CH₂)ₘ—NR²R³, —O(CH₂)ₙ—NR²R³, fluoro, chloro or 5- or 6-membered heteroaryl optionally substituted by C₁₋₃alkyl;

W¹ is hydrogen, halogen or C₁₋₃alkoxy;

Z is C₁₋₃alkylene, C₂₋₃alkenylene or a bond;

Q is phenyl, pyridyl or a 9-membered bicyclic heteroaryl group;

T is halogen, COOR¹, COR¹, C₁₋₆alkyl, —(CH₂)ₘ—NR²R³, or a 5- to 10-membered heteroaryl group optionally substituted by C₁₋₃alkyl; or when Z is C₁₋₃alkylene or C₂₋₃alkenylene, then T may be hydrogen;

T¹ and T² are independently selected from hydrogen, halogen and hydroxy;

R¹ is C₁₋₆alkyl or phenyl or a 5- or 6-membered heteroaryl or heterocyclyl group;

R² and R³ together with the nitrogen to which they are attached form a 5- to 7-membered heterocyclic ring optionally containing an additional heteroatom selected from O, S and NR⁴, and optionally substituted by 1 or 2 groups independently selected from C₁₋₃alkyl, fluoro and hydroxy;

m is 0, 1, 2 or 3; and

n is 2 or 3;

provided that the compound is not:
1-Benzo[b]thiophen-2-yl-3-(2-methylphenyl)urea,
4-[3-(2-Chlorophenyl)ureido]benzoic acid ethyl ester,
4-[3-(4-Methylsulfanylphenyl)ureido]benzoic acid ethyl ester,
4-[3-(4-Chlorophenyl)ureido]benzoic acid ethyl ester,
1-(3-Chlorophenyl)-3-(4-ethoxyphenyl)urea,
4-[3-(3-Chlorophenyl)ureido]benzoic acid ethyl ester,
1,3-Bis(4-acetylphenyl)urea,
4-[3-(4-Fluorophenyl)ureido]benzoic acid ethyl ester,
1-(4-Fluorophenyl)-3-(4-methoxyphenyl)urea,
1-(4-Acetylphenyl)-3-(3-chlorophenyl)urea,
1-(4-Acetylphenyl)-3-(4-chlorophenyl)urea,
1-(4-Chlorophenyl)-3-(4-ethoxyphenyl)urea,
1-(4-Acetylphenyl)-3-(4-fluorophenyl)urea,
4-[3-(4-Fluorophenyl)ureido]benzoic acid methyl ester,
4-[3-(3-Fluorophenyl)ureido]benzoic acid ethyl ester,
4-[3-(2-Fluorophenyl)ureido]benzoic acid ethyl ester,
1-(3-Ethoxyphenyl)-3-(4-fluorophenyl)urea,
1-(4-Chlorophenyl)-3-(4-trifluoromethoxyphenyl)urea,
1-(3-Acetylphenyl)-3-[2-(4-chlorophenyl)ethyl]urea, or
1-(4-Chlorophenyl)-3-[4-(morpholine-4-carbonyl)phenyl]urea.

The molecular weight of the compounds of formula (Ia) is preferably less than 800, more preferably less than 600.

As far as they are appropriate the preferences given for variables in the compounds of formula (I) recited above are also applicable to the compounds of formula (Ia).

In the compounds of formula (Ia):

Particular examples of 5- or 6-membered heteroaryl groups that Y may represent include thienyl, thiazolyl and thiadiazolyl.

Particular examples of 9-membered bicyclic heteroaryl groups that Y and Q may represent include benzothienyl and benzothiazolyl, especially benzothien-2-yl and benzothiazol-2-yl.

A specific group of compounds of formula (Ia) which may be mentioned are those where Y is phenyl.

When Y is phenyl, W is preferably COOR¹ especially COOEt, or COR¹, C₁₋₆alkoxy, fluoro, chloro, C₁₋₃alkoxyC₁₋₃alkoxy, —(CH₂)ₘ—NR²R³, —O(CH₂)ₙ—NR²R³, or 5- or 6-membered heteroaryl optionally substituted by C₁₋₃alkyl. Particular W groups which may be mentioned are chloro, C₁₋₃alkoxyC₁₋₃alkoxy, —(CH₂)ₘ—NR²R³ and —O(CH₂)ₙ—NR²R³ where —NR²R³, is preferably morpholinyl.

Heteroaryl groups which W may represent include 5- or 6-membered heteroaryl groups containing 1 or 2 nitrogen atoms such as pyrazole, pyrrole, imidazole, pyrimidine or pyridine.

W¹ is preferably hydrogen, halogen or C₁₋₃alkoxy, more preferably hydrogen.

W¹ is preferably hydrogen.

Z is preferably C₂alkylene, C₂alkenylene or a bond, more preferably C₂alkylene or a bond, especially a bond.

A specific group of compounds of formula (Ia) which may be mentioned are those where Q is phenyl.

A group of compounds of formula (Ia) which may be mentioned are those where T is halogen, COOR¹, COR¹, C₁₋₆alkyl, C₁₋₆alkylthio, or a 5- or 6-membered heteroaryl group optionally substituted by C₁₋₃alkyl; or when Z is C₁₋₃alkylene or C₂₋₃alkenylene, then T may be hydrogen.

T is preferably halogen, COOR¹, COR¹, —(CH₂)ₘ—NR²R³ optionally substituted by 1 or 2 groups independently selected from fluoro and hydroxy, or a 5- to 10-membered heteroaryl group optionally substituted by C₁₋₃ alkyl, e.g. a 5- or 6-membered heteroaryl group containing 1 or 2 nitrogen atoms such as pyrazole, pyrrole, imidazole, pyrimidine or pyridine, or thiazole, thiadiazole, oxazole or 3,4-dihydro-1H-isoquinolin-2-yl.

T¹ and T² are preferably hydrogen, halogen or hydroxy, more preferably hydrogen or halogen.

T² is preferably hydrogen.

A specific group of compounds which may be mentioned are those where T is —(CH₂)ₘ—NR²R³, T⁴ is halogen, e.g. fluoro, and T² is hydrogen.

When T is —(CH₂)ₘ—NR²R³, m is preferably 0 and R² and R³ together with the nitrogen to which they are attached preferably form a 5- to 7-membered heterocyclic ring, e.g. a piperidine ring, optionally substituted by 1 or 2 groups independently selected from C₁₋₃alkyl, fluoro and hydroxy, e.g. methyl.

W and T are preferably different.

Preferably at least one of Y and Q is phenyl.

A group of compounds of formula (Ia) which may be mentioned are those where R¹ is C₁₋₆alkyl or phenyl or a 5- or 6-membered heteroaryl group.

The present invention also provides a compound selected from:
2-[3-(4-Fluorophenyl)ureido]-4-methylthiazole-5-carboxylic acid ethyl ester
2-[3-(3-Fluorophenyl)ureido]-4-methylthiazole-5-carboxylic acid ethyl ester
2-[3-(4-Ethoxycarbonylphenyl)ureido]-4-methylthiazole-5-carboxylic acid ethyl ester
4-Methyl-2-[3-(4-methylsulfanylphenyl)ureido]thiazole-5-carboxylic acid ethyl ester
1-(4-Acetylphenyl)-3-benzo[b]thiophen-2-ylurea
1-Benzo[b]thiophen-2-yl-3-(4-methanesulfonylphenyl)urea
4-[3-(4-Fluoro-2-methylphenyl)ureido]benzoic acid ethyl ester
4-[3-(2,4,6-Trifluorophenyl)ureido]benzoic acid ethyl ester
4-[3-(2,4-Difluorophenyl)ureido]benzoic acid ethyl ester
4-[3-(3,4-Difluorophenyl)ureido]benzoic acid ethyl ester
4-[3-(2-Chloro-4-fluorophenyl)ureido]benzoic acid ethyl ester
4-[3-(4-Fluoro-3-methylphenyl)ureido]benzoic acid ethyl ester
4-[3-(3-Chloro-4-fluorophenyl)ureido]benzoic acid ethyl ester
4-[3-(4-Fluoro-3-methoxyphenyl)ureido]benzoic acid ethyl ester
1-(4-Ethoxyphenyl)-3-(4-fluorophenyl)urea
4-[3-(4-Fluorophenyl)ureido]-3-methylbenzoic acid methyl ester
4-[3-(4-Fluorophenyl)ureido]-3-hydroxybenzoic acid methyl ester
1-(2-Thiophen-2-ylethyl)-3-(4-methylphenyl)urea
1-(4-Methoxyphenyl)-3-(2-thiophen-2-ylethyl)urea
1-(2-Thiophen-2-ylethyl)-3-(4-trifluoromethoxyphenyl)urea
1-(4-Difluoromethoxyphenyl)-3-(2-thiophen-2-ylethyl)urea
1-(4-Ethylphenyl)-3-(2-thiophen-2-ylethyl)urea
1-(2-Thiophen-2-ylethyl)-3-(4-trifluoromethylphenyl)urea
1-(3-Chlorophenyl)-3-(2-thiophen-2-ylethyl)urea
1-(4-Butylphenyl)-3-(2-thiophen-2-ylethyl)urea
1-(4-Acetylphenyl)-3-(2-thiophen-2-ylethyl)urea
1-(3-Ethylphenyl)-3-(2-thiophen-2-ylethyl)urea
1-(4-Fluorophenyl)-3-(2-thiophen-2-ylethyl)urea
1-(4-Chlorophenyl)-3-(2-thiophen-2-ylethyl)urea
1-(4-Methylsulfanylphenyl)-3-(2-thiophen-2-ylethyl)urea
1-(4-Isopropylphenyl)-3-(2-thiophen-2-ylethyl)urea
4-(3-Benzothiazol-6-ylureido)benzoic acid ethyl ester
4-[3-(4-Imidazol-1-ylphenyl)ureido]benzoic acid ethyl ester
4-[3-(6-Fluorobenzothiazol-2-yl)ureido]benzoic acid ethyl ester
5-[3-(4-Ethoxycarbonylphenyl)ureido]furan-2-carboxylic acid methyl ester
4-[3-(1H-Indol-6-ylureido]benzoic acid ethyl ester
4-[3-(3-Methoxycarbonylphenyl)ureido]benzoic acid ethyl ester
2-[3-(4-Ethoxycarbonylphenyl)ureido]thiophene-3-carboxylic acid methyl ester
4-{3-[2-(1-Methyl-1H-pyrrol-2-yl)ethyl]ureido}benzoic acid ethyl ester
4-[3-(6-Methoxypyridin-3-yl)ureido]benzoic acid ethyl ester
6-[3-(4-Ethoxycarbonylphenyl)ureido]nicotinic acid methyl ester
4-[3-(6-Chloropyridin-3-yl)ureido]benzoic acid ethyl ester
4-[3-(4-Carboxymethylphenyl)ureido]benzoic acid ethyl ester
4-[3-(1H-Indol-5-yl)ureido]benzoic acid ethyl ester
1-(4-Fluorophenyl)-3-(4-morpholin-4-ylphenyl)urea
1-Benzothiazol-6-yl-3-(4-fluorophenyl)urea
1-(4-Fluorophenyl)-3-(4-imidazol-1-ylphenyl)urea
6-[3-(4-Fluorophenyl)ureido]nicotinic acid methyl ester
1-(6-Chlorobenzothiazol-2-yl)-3-(4-fluorophenyl)urea
1-(6-Fluorobenzothiazol-2-yl)-3-(4-fluorophenyl)urea
1-(4,6-Difluorobenzothiazol-2-yl)-3-(4-fluorophenyl)urea
1-(4-Fluorophenyl)-3-(6-methoxybenzothiazol-2-yl)urea
1-(4-Fluorophenyl)-3-[3-(2-methylpyrimidin-4-yl)phenyl]urea
3-[3-(4-Fluorophenyl)ureido]benzoic acid methyl ester
1-(4-Fluorophenyl)-3-(2-fluorophenyl)urea
3-[3-(4-Fluorophenyl)ureido]benzoic acid ethyl ester
1-(4-Fluoro-3-methylphenyl)-3-(4-fluorophenyl)urea
4-{3-[3-(2-Methylpyrimidin-4-yl)phenyl]ureido}benzoic acid ethyl ester
4-[3-(1-Oxoindan-5-yl)ureido]benzoic acid ethyl ester
4-[3-(6-Morpholin-4-ylpyridin-3-yl)ureido]benzoic acid ethyl ester
2-[3-(4-Ethoxycarbonylphenyl)ureido]thiazole-5-carboxylic acid methyl ester
4-[3-(3-Ethoxycarbonylphenyl)ureido]benzoic acid ethyl ester
4-[3-(4-Ethoxycarbonylphenyl)ureido]benzoic acid propyl ester
4-[3-(4-Ethoxycarbonylphenyl)ureido]benzoic acid pentyl ester
4-[3-(4-Ethoxycarbonylphenyl)ureido]benzoic acid isobutyl ester
4-[3-(4-Ethoxycarbonylphenyl)ureido]benzoic acid phenyl ester
4-{3-[4-(1,1,2,2-Tetrafluoroethoxy)phenyl]ureido}benzoic acid ethyl ester
4-[3-(3-Oxazol-5-ylphenyl)ureido]benzoic acid ethyl ester
4-[3-(4-Ethoxycarbonylmethylphenyl)ureido]benzoic acid ethyl ester
4-[3-(4-[1,2,3]Thiadiazol-4-ylphenyl)ureido]benzoic acid ethyl ester
4-[3-(4-Propionylphenyl)ureido]benzoic acid ethyl ester
4-[3-(4-Acetylphenyl)ureido]benzoic acid ethyl ester
4-[3-(4-Benzoylphenyl)ureido]benzoic acid ethyl ester
4-{3-[4-(4,5-Dihydrooxazol-2-yl)phenyl]ureido}benzoic acid ethyl ester
4-{3-[4-(2-Methylpyrimidin-4-yl)phenyl]ureido}benzoic acid ethyl ester
1-(4-Fluorophenyl)-3-(4-pyrrol-1-ylphenyl)urea
1-(4-Fluorophenyl)-3-(2-methylbenzothiazol-5-yl)urea
1-(4-Fluorophenyl)-3-(3-oxazol-5-ylphenyl)urea
1-(4-Fluorophenyl)-3-(4-propionylphenyl)urea
1-(4-Fluorophenyl)-3-[4-(2-methylpyrimidin-4-yl)phenyl]urea
4-[3-(4-Ethoxycarbonylphenyl)ureido]benzoic acid butyl ester
2-[3-(4-Ethoxycarbonylphenyl)ureido]-4-methylpyrimidine-5-carboxylic acid ethyl ester
4-[3-(4-Oxazol-5-ylphenyl)ureido]benzoic acid ethyl ester
2-Chloro-4-[3-(4-ethoxycarbonylphenyl)ureido]benzoic acid ethyl ester
4-[3-(4-Ethoxycarbonylphenyl)ureido]-2-methoxybenzoic acid ethyl ester
4-[3-(4-Ethoxycarbonylphenyl)ureido]-3-methoxybenzoic acid ethyl ester
6-[3-(4-Ethoxycarbonylphenyl)ureido]nicotinic acid ethyl ester
4-[3-(4-Fluorophenyl)ureido]-3-hydroxybenzoic acid ethyl ester
4-[3-(3-Acetylphenyl)ureido]benzoic acid ethyl ester
4-[3-(4-Butyrylphenyl)ureido]benzoic acid ethyl ester
4-{3-[4-(1H-Pyrazol-3-yl)phenyl]ureido}benzoic acid ethyl ester
4-[3-(4-Fluorophenyl)ureido]benzoic acid propyl ester
4-[3-(4-Fluorophenyl)ureido]benzoic acid pentyl ester
4-[3-(4-Fluorophenyl)ureido]benzoic acid isobutyl ester
4-[3-(4-Fluorophenyl)ureido]benzoic acid phenyl ester
{4-[3-(4-Fluorophenyl)ureido]phenyl}acetic acid ethyl ester
1-(4-Benzoylphenyl)-3-(4-fluorophenyl)urea
1-(4-Butyrylphenyl)-3-(4-fluorophenyl)urea
4-[3-(4-Fluorophenyl)ureido]benzoic acid butyl ester
2-Chloro-4-[3-(4-fluorophenyl)ureido]benzoic acid ethyl ester
1-[2-(3-Fluorophenyl)ethyl]-3-(4-isopropylphenyl)urea
1-[2-(2-Fluorophenyl)ethyl]-3-(4-isopropylphenyl)urea
1-[2-(3-Fluorophenyl)ethyl]-3-(4-trifluoromethylphenyl)urea
1-(4-Isopropylphenyl)-3-thiazol-2-ylurea
1-(4-Acetylphenyl)-3-(4-bromophenyl)urea
1-[2-(4-Chlorophenyl)ethyl]-3-[3-(2-methylpyrimidin-4-yl)phenyl]urea
1-(4-Chlorophenyl)-3-(3-pyrrol-1-ylphenyl)urea
1-(4-Chlorophenyl)-3-(4-pyrrol-1-ylphenyl)urea
1-(4-Chlorophenyl)-3-[3-(2-methylpyrimidin-4-yl)phenyl]urea
1-(4-Chlorophenyl)-3-[4-(3,4-dihydro-1H-isoquinolin-2-yl)-3-fluorophenyl]urea
1-(3,4-Dichlorophenyl)-3-(3-fluoro-4-piperidin-1-ylphenyl)urea
1-(4-Chlorophenyl)-3-[3-(6-pyrrolidin-1-ylpyridin-2-yl)phenyl]urea
1-(4-Azepan-1-yl-3-fluorophenyl)-3-(4-chlorophenyl)urea
1-(4-Chlorophenyl)-3-(3-fluoro-4-pyrrolidin-1-ylphenyl)urea
1-(3-Fluoro-4-piperidin-1-ylphenyl)-3-[4-(2-morpholin-4-ylethoxy)phenyl]urea
1-(3-Fluoro-4-piperidin-1-ylphenyl)-3-[4-(2-methoxyethoxy)phenyl]urea
1-(4-Chlorophenyl)-3-[3-(2-isopropylpyrimidin-4-yl)phenyl]urea
1-(4-Chlorophenyl)-3-(3-fluoro-4-[1,4]oxazepan-4-ylphenyl)urea
1-(3-Fluoro-4-piperidin-1-ylphenyl)-3-[4-(morpholine-4-carbonyl)phenyl]urea
1-(3-Fluoro-4-piperidin-1-ylphenyl)-3-(4-pyrrol-1-ylphenyl)urea
4-[3-(3-Fluoro-4-piperidin-1-ylphenyl)ureido]benzoic acid ethyl ester
1-(3-Fluoro-4-piperidin-1-ylphenyl)-3-[3-(2-methoxyethoxy)phenyl]urea
1-(3-Fluoro-4-piperidin-1-ylphenyl)-3-[3-(2-morpholin-4-ylethoxy)phenyl]urea
1-(4-Chlorophenyl)-3-(4-pyridin-3-ylphenyl)urea
1-(4-Chlorophenyl)-3-[3-(6-methylpyrimidin-4-yl)phenyl]urea
1-(4-Chlorophenyl)-3-[3-fluoro-4-(3-hydroxypiperidin-1-yl)phenyl]urea
1-(4-Chlorophenyl)-3-(4-pyridin-2-yl-phenyl)urea
1-(4-Chlorophenyl)-3-(4-pyridin-4-ylphenyl)urea
1-(4-Chlorophenyl)-3-[3-(2-piperidin-1-ylpyrimidin-4-yl)phenyl]urea
1-(3-Fluoro-4-piperidin-1-ylphenyl)-3-[4-(2-morpholin-4-ylethyl)phenyl]urea
1-(3-Fluoro-4-piperidin-1-ylphenyl)-3-(4-morpholin-4-ylmethylphenyl)urea
1-(2,3-Dihydrobenzofuran-6-yl)-3-(3-fluoro-4-piperidin-1-ylphenyl)urea
1-(3,5-Dimethoxyphenyl)-3-(3-fluoro-4-piperidin-1-ylphenyl)urea
1-(4-Chlorophenyl)-3-(3-pyrazol-1-ylphenyl)urea
1-(4-Chlorophenyl)-3-[3-(3,4,5,6-tetrahydro-2H-[1,2′]bipyridinyl-6′-yl)phenyl]urea
1-(4-Morpholin-4-ylmethylphenyl)-3-(4-pyrrol-1-ylphenyl)urea
1-(4-Morpholin-4-ylmethylphenyl)-3-(3-pyrrol-1-ylphenyl)urea
1-(4-Chlorophenyl)-3-[4-(4,4-difluoropiperidin-1-yl)-3-fluorophenyl]urea
1-(4-Butyrylphenyl)-3-[4-(morpholine-4-carbonyl)phenyl]urea
1-(1-Methyl-1H-indazol-5-yl)-3-(4-morpholin-4-ylmethylphenyl)urea
1-(4-Morpholin-4-ylmethylphenyl)-3-(4-pyrazol-1-ylphenyl)urea
1-(2,3-Dihydrobenzo[1,4]dioxin-6-yl)-3-(3-fluoro-4-piperidin-1-ylphenyl)urea
1-(3,5-Dichlorophenyl)-3-(3-fluoro-4-piperidin-1-ylphenyl)urea
1-(3-Chloro-4-fluorophenyl)-3-(3-fluoro-4-piperidin-1-ylphenyl)urea
1-(4-Ethylphenyl)-3-(3-fluoro-4-piperidin-1-ylphenyl)urea
1-[2-(4-Chlorophenyl)ethyl]-3-[3-(2-methyl-2H-pyrazol-3-yl)phenyl]urea
1-(4-Chlorophenyl)-3-[3-fluoro-4-(4-hydroxypiperidin-1-yl)phenyl]urea
1-(4-Chlorophenyl)-3-[3-fluoro-4-(3-methylpiperidin-1-yl)phenyl]urea
1-Benzo[1,3]dioxol-5-yl-3-(3-fluoro-4-piperidin-1-ylphenyl)urea
1-(4-Chlorophenyl)-3-[3-fluoro-4-(2-methylpiperidin-1-yl)phenyl]urea
1-(3-Fluoro-4-piperidin-1-ylphenyl)-3-(4-methoxyphenyl)urea
1-(4-Chloro-2-hydroxyphenyl)-3-(3-fluoro-4-piperidin-1-ylphenyl)urea
1-(3-Fluoro-4-piperidin-1-ylphenyl)-3-[3-(2-methylpyrimidin-4-yl)phenyl]urea
1-(4-Chlorophenyl)-3-[3-fluoro-4-(4-trifluoromethylpiperidin-1-yl)phenyl]urea
1-(4-Chlorophenyl)-3-[3-fluoro-4-(4-methylpiperidin-1-yl)phenyl]urea
1-(3-Fluoro-4-piperidin-1-ylphenyl)-3-(4-phenoxyphenyl)urea
1-(3-Fluoro-4-piperidin-1-ylphenyl)-3-(3-phenoxyphenyl)urea
1-(4-Fluorophenyl)-3-(3-fluoro-4-piperidin-1-ylphenyl)urea
1-(3-Fluoro-4-piperidin-1-ylphenyl)-3-(3-methoxyphenyl)urea
1-(4-Cyanophenyl)-3-(3-fluoro-4-piperidin-1-ylphenyl)urea
1-(4-Chlorophenyl)-3-(4-morpholin-4-ylmethylphenyl)urea
1-(4-Chloro-3-trifluoromethylphenyl)-3-(3-fluoro-4-piperidin-1-ylphenyl)urea
1-(3-Fluoro-4-piperidin-1-ylphenyl)-3-(4-trifluoromethylphenyl)urea
1-(3-Chlorophenyl)-3-(3-fluoro-4-piperidin-1-ylphenyl)urea
1-(4-Chlorophenyl)-3-(3,4,5,6-tetrahydro-2H-[1,2′]bipyridinyl-5′-yl)urea
1-(4-Chlorophenyl)-3-(3-dimethylaminophenyl)urea
1-(4-Chlorophenyl)-3-(3-fluoro-4-morpholin-4-ylphenyl)urea
1-[2-(4-Chlorophenyl)ethyl]-3-(3-pyrrol-1-ylphenyl)urea
1-(3,5-Dichlorophenyl)-3-(3,4,5,6-tetrahydro-2H-[1,2′]bipyridinyl-5′-yl)urea
1-(3-Chlorophenyl)-3-(3,4,5,6-tetrahydro-2H-[1,2′]bipyridinyl-5′-yl)urea
1-(3,5-Bis-trifluoromethylphenyl)-3-(3,4,5,6-tetrahydro-2H-[1,2′]bipyridinyl-5′-yl)urea
1-(4-Acetylphenyl)-3-[4-(morpholine-4-carbonyl)phenyl]urea
1-(4-Acetylphenyl)-3-[3-(6-methoxypyridin-2-yl)phenyl]urea
1-(4-Acetylphenyl)-3-(4-morpholin-4-ylmethylphenyl)urea
1-(4-Chlorophenyl)-3-(3-fluoro-4-piperidin-1-ylphenyl)urea
1-(3-Chloro-4-morpholin-4-ylphenyl)-3-(4-chlorophenyl)urea
1-(4-Chlorophenyl)-3-(4-piperidin-1-ylphenyl)urea
1-(4-Acetylphenyl)-3-(3,4,5,6-tetrahydro-2H-[1,2′]bipyridinyl-5′-yl)urea
1-(4-Butyrylphenyl)-3-(4-piperidin-1-ylphenyl)urea
1-[2-(4-Chlorophenyl)ethyl]-3-(4-morpholin-4-ylmethylphenyl)urea
1-(4-Chlorophenyl)-3-(1-methyl-1H-indazol-5-yl)urea
1-(4-Chlorophenyl)-3-[3-(2-pyrrolidin-1-ylpyrimidin-4-yl)phenyl]urea
1-(4-Chlorophenyl)-3-(4-pyrazol-1-ylphenyl)urea
1-[2-(4-Chlorophenyl)ethyl]-3-[4-(morpholine-4-carbonyl)phenyl]urea

and pharmaceutically acceptable salts thereof.

As used herein, unless stated otherwise, “alkyl” as well as other groups having the prefix “alk” such as, for example, alkoxy, alkylene, alkenyl, alkynyl, and the like, means carbon chains which may be linear or branched or combinations thereof. Examples of alkyl groups include methyl, ethyl, propyl, isopropyl, butyl, sec- and tert-butyl, pentyl, hexyl and the like. “Alkenyl” and other like terms include carbon chains having at least one unsaturated carbon-carbon bond. As used herein, for example, “C₁₋₆alkyl” is used to mean an alkyl having 1-6 carbons, i.e. 1, 2, 3, 4, 5 or 6 carbons in a straight or branched configuration.

C₁₋₃-Fluoroalkyl and C₁₋₃-fluoroalkoxy include groups where one or more hydrogen atoms are replaced by fluorine, e.g. —CH₂F, —CHF₂, —CF₃, —OCH₂F, —OCHF₂, —OCF₃ and —OCF₂CHF₂.

The term “halogen” includes fluorine, chlorine, bromine, and iodine atoms, especially fluorine and chlorine atoms.

Unless otherwise stated, the term “heterocyclyl” includes 5- to 7-membered, particularly 5- and 6-membered, saturated and partially saturated rings containing one or two heteroatoms chosen from oxygen, sulfur, and nitrogen. The heteroatoms are not directly attached to one another. Examples of heterocyclic rings include oxetane, tetrahydrofuran, tetrahydropyran, oxepane, oxocane, thietane, tetrahydrothiophene, tetrahydrothiopyran, thiepane, thiocane, azetidine, pyrrolidine, piperidine, azepane, azocane, [1,3]dioxane, oxazolidine, piperazine, morpholine, 4,5-dihydrooxazole and the like. Other examples of heterocyclic rings include the oxidised forms of the sulfur-containing rings. Thus, tetrahydrothiophene 1-oxide, tetrahydrothiophene 1,1-dioxide, tetrahydrothiopyran 1-oxide, and tetrahydrothiopyran 1,1-dioxide are also considered to be heterocyclic rings.

Unless otherwise stated, the term “heteroaryl” includes mono- and bicyclic 5- to 10-membered heteroaryl rings containing 1-4 heteroatoms chosen from oxygen, sulfur, and nitrogen. Examples of such heteroaryl rings are furyl, thienyl, pyrrolyl, pyrazolyl, imidazolyl, oxazolyl, isoxazolyl, thiazolyl, isothiazolyl, triazolyl, oxadiazolyl, thiadiazolyl, tetrazolyl, pyridinyl, pyridazinyl, pyrimidinyl, pyrazinyl, and triazinyl. Bicyclic heteroaryl groups include bicyclic heteroaromatic groups where a 5- or 6-membered heteroaryl ring is fused to a phenyl or another heteroaromatic group. Examples of such bicyclic heteroaromatic rings are benzofuran, benzothiophene, indole, benzoxazole, benzothiazole, indazole, benzimidazole, benzotriazole, quinoline, isoquinoline, quinazoline, quinoxaline and purine. Bicyclic heteroaryl groups also include groups formed from a fused aromatic ring and a saturated or partially saturated ring, for example 3,4-dihydro-1H-isoquinoline or 2,3-dihydrobenzofuran.

The above formulae are shown without a definitive stereochemistry at certain positions. The present invention includes all stereoisomers, e.g. geometric isomers, optical isomers, diastereoisomers, etc, and pharmaceutically acceptable salts thereof, except where specifically drawn or stated otherwise. Further, mixtures of stereoisomers as well as isolated specific stereoisomers are also included, except where specifically drawn or stated otherwise. During the course of the synthetic procedures used to prepare such compounds, or in using racemization or epimerization procedures known to those skilled in the art, the products of such procedures can be a mixture of stereoisomers. The different isomeric forms may be separated or resolved from one another by conventional methods, or any given isomer may be obtained by conventional synthetic methods or by stereospecific or asymmetric syntheses. When an isomeric form of a compound is provided substantially free from other isomers, it will preferably contain less than 5% w/w, more preferably less than 2% w/w and especially less than 1% w/w of the other isomers.

When a tautomer of the compound of the above formulae exists, the present invention includes any possible tautomers and pharmaceutically acceptable salts thereof, and mixtures thereof, except where specifically drawn or stated otherwise.

When the compound of the above formulae and pharmaceutically acceptable salts thereof exist in the form of solvates or polymorphic forms, the present invention includes any possible solvates and polymorphic forms. A type of a solvent that forms the solvate is not particularly limited so long as the solvent is pharmacologically acceptable. For example, water, ethanol, propanol, acetone or the like can be used.

The term “pharmaceutically acceptable salts” refers to salts prepared from pharmaceutically acceptable non-toxic bases or acids. When the compound of the present invention is acidic, its corresponding salt can be conveniently prepared from pharmaceutically acceptable non-toxic bases, including inorganic bases and organic bases. Salts derived from such inorganic bases include aluminum, ammonium, calcium, copper (ic and ous), ferric, ferrous, lithium, magnesium, manganese (ic and ous), potassium, sodium, zinc and the like salts. Salts derived from pharmaceutically acceptable organic non-toxic bases include salts of primary, secondary, and tertiary amines, as well as cyclic amines and substituted amines such as naturally occurring and synthesized substituted amines. Other pharmaceutically acceptable organic non-toxic bases from which salts can be formed include ion exchange resins such as, for example, arginine, betaine, caffeine, choline, N′,N′-dibenzylethylenediamine, diethylamine, 2-diethylaminoethanol, 2-dimethylaminoethanol, ethanolamine, ethylenediamine, N-ethylmorpholine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylameine, trimethylamine, tripropylamine, tromethamine and the like.

When the compound of the invention is basic, its corresponding salt can be conveniently prepared from pharmaceutically acceptable non-toxic acids, including inorganic and organic acids. Such acids include, for example, acetic, benzenesulfonic, benzoic, camphorsulfonic, citric, ethanesulfonic, fumaric, gluconic, glutamic, hydrobromic, hydrochloric, isethionic, lactic, maleic, malic, mandelic, methanesulfonic, mucic, nitric, pamoic, pantothenic, phosphoric, succinic, sulfuric, tartaric, p-toluenesulfonic acid and the like.

Since the compounds of formula (I) are intended for pharmaceutical use they are preferably provided in substantially pure form, for example at least 60% pure, more suitably at least 75% pure especially at least 98% pure (% are on a weight for weight basis).

In accordance with this invention, the compounds of formula (I) can be prepared as illustrated in the schemes below:

Compounds of formula (I) can be readily prepared by combining an amine of formula (II) with an isocyanate of formula (III) in a suitable solvent, at a temperature of typically between 20° C. and 100° C. (Scheme 1). An example of a suitable solvent is toluene. Compounds of formulae (II) and (III) are generally commercially available or readily synthesised using known techniques.

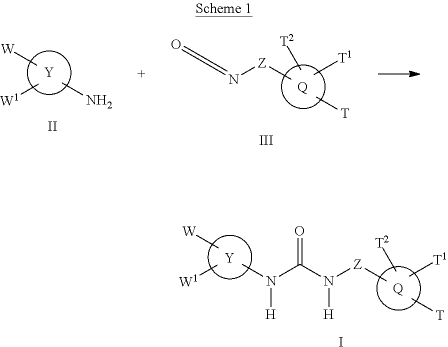

Compounds of formula (I) can alternatively be prepared by combining an amine of formula (IV) with an isocyanate of formula (V) using the conditions described above (Scheme 2). Compounds of formulae (IV) and (V) are generally commercially available or readily synthesised using known techniques.

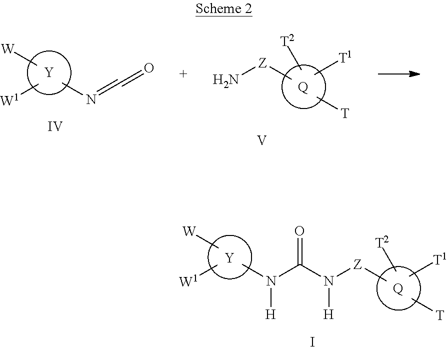

Synthesis of non-commercial isocyanates of formula (III) or (IV) can be achieved, for example, from an acid chloride of formulae (VI) or (VII) (FIG. 1) by treatment with sodium azide in a suitable solvent such as tetrahydrofuran and water. The resulting acylazide is then heated in a suitable solvent such as toluene. Acid chlorides of formulae (VI) and (VII) are typically commercially available or readily synthesised for the corresponding carboxylic acid using known techniques. Examples of isocyantes that may be synthesised using this process include compounds of formula (IV) where Y=benzothiophene, and compounds of formula (III) where Z=alkenylene. The isocyantes can be used in situ and reacted with a suitable amine to provide compounds of formula (I) as described above.

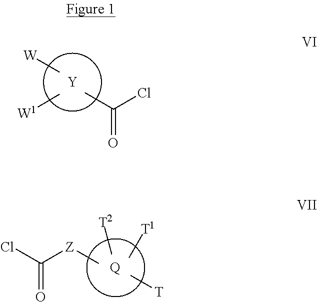

Amines of formulae (II) and (V) may also be prepared from compounds of formulae (VI) and (VII). The corresponding isocyanates are prepared under condition described above and then hydrolysed using water to give the corresponding amines of formulae (II) and (V).

Further details for the preparation of the compounds of formula (I) are found in the examples.

The compounds of formula (I) may be prepared singly or as compound libraries comprising at least 2, for example 5 to 1,000 compounds and more preferably 10 to 100 compounds of formula (I). Compound libraries may be prepared by a combinatorial “split and mix” approach or by multiple parallel synthesis using either solution or solid phase chemistry, using procedures known to those skilled in the art.

Any novel intermediates of use in the preparation of the compounds of formula (I) are also encompassed by the present invention.

During the synthesis of the compounds of formula (I), labile functional groups in the intermediate compounds, e.g. hydroxy, carboxy and amino groups, may be protected. The protecting groups may be removed at any stage in the synthesis of the compounds of formula (I) or may be present on the final compound of formula (I). A comprehensive discussion of the ways in which various labile functional groups may be protected and methods for cleaving the resulting protected derivatives is given in for example, Protective Groups in Organic Chemistry, T. W. Greene and P. G. M. Wuts, (1991) Wiley-Interscience, New York, 2^{d} edition.

As indicated above the compounds of formula (I) are useful for the treatment of conditions associated with the CB-1 receptor, in particular obesity. For such use the compounds of formula (I) will generally be administered in the form of a pharmaceutical composition.

Certain of the compounds of formula (I) have not previously been disclosed as having pharmaceutical utility.

The invention also provides a pharmaceutical composition comprising a compound of formula (Ia) or a pharmaceutically acceptable salt thereof, in combination with a pharmaceutically acceptable carrier.

The invention also provides a pharmaceutical composition comprising a compound selected from:
2-[3-(4-Fluorophenyl)ureido]-4-methylthiazole-5-carboxylic acid ethyl ester
2-[3-(3-Fluorophenyl)ureido]-4-methylthiazole-5-carboxylic acid ethyl ester
2-[3-(4-Ethoxycarbonylphenyl)ureido]-4-methylthiazole-5-carboxylic acid ethyl ester
4-Methyl-2-[3-(4-methylsulfanylphenyl)ureido]thiazole-5-carboxylic acid ethyl ester
1-(4-Acetylphenyl)-3-benzo[b]thiophen-2-ylurea
1-Benzo[b]thiophen-2-yl-3-(4-methanesulfonylphenyl)urea
4-[3-(4-Fluoro-2-methylphenyl)ureido]benzoic acid ethyl ester
4-[3-(2,4,6-Trifluorophenyl)ureido]benzoic acid ethyl ester
4-[3-(2,4-Difluorophenyl)ureido]benzoic acid ethyl ester
4-[3-(3,4-Difluorophenyl)ureido]benzoic acid ethyl ester
4-[3-(2-Chloro-4-fluorophenyl)ureido]benzoic acid ethyl ester 4-[3-(4-Fluoro-3-methylphenyl)ureido]benzoic acid ethyl ester
4-[3-(3-Chloro-4-fluorophenyl)ureido]benzoic acid ethyl ester
4-[3-(4-Fluoro-3-methoxyphenyl)ureido]benzoic acid ethyl ester
1-(4-Ethoxyphenyl)-3-(4-fluorophenyl)urea
4-[3-(4-Fluorophenyl)ureido]-3-methylbenzoic acid methyl ester
4-[3-(4-Fluorophenyl)ureido]-3-hydroxybenzoic acid methyl ester
1-(2-Thiophen-2-ylethyl)-3-(4-methylphenyl)urea
1-(4-Methoxyphenyl)-3-(2-thiophen-2-ylethyl)urea
1-(2-Thiophen-2-ylethyl)-3-(4-trifluoromethoxyphenyl)urea
1-(4-Difluoromethoxyphenyl)-3-(2-thiophen-2-ylethyl)urea
1-(4-Ethylphenyl)-3-(2-thiophen-2-ylethyl)urea
1-(2-Thiophen-2-ylethyl)-3-(4-trifluoromethylphenyl)urea
1-(3-Chlorophenyl)-3-(2-thiophen-2-ylethyl)urea
1-(4-Butylphenyl)-3-(2-thiophen-2-ylethyl)urea
1-(4-Acetylphenyl)-3-(2-thiophen-2-ylethyl)urea
1-(3-Ethylphenyl)-3-(2-thiophen-2-ylethyl)urea
1-(4-Fluorophenyl)-3-(2-thiophen-2-ylethyl)urea
1-(4-Chlorophenyl)-3-(2-thiophen-2-ylethyl)urea
1-(4-Methylsulfanylphenyl)-3-(2-thiophen-2-ylethyl)urea
1-(4-Isopropylphenyl)-3-(2-thiophen-2-ylethyl)urea
4-(3-Benzothiazol-6-ylureido)benzoic acid ethyl ester
4-[3-(4-Imidazol-1-ylphenyl)ureido]benzoic acid ethyl ester
4-[3-(6-Fluorobenzothiazol-2-yl)ureido]benzoic acid ethyl ester
5-[3-(4-Ethoxycarbonylphenyl)ureido]furan-2-carboxylic acid methyl ester
4-[3-(1H-Indol-6-yl)ureido]benzoic acid ethyl ester
4-[3-(3-Methoxycarbonylphenyl)ureido]benzoic acid ethyl ester
2-[3-(4-Ethoxycarbonylphenyl)ureido]thiophene-3-carboxylic acid methyl ester
4-{3-[2-(1-Methyl-1H-pyrrol-2-yl)ethyl]ureido}benzoic acid ethyl ester
4-[3-(6-Methoxypyridin-3-yl)ureido]benzoic acid ethyl ester
6-[3-(4-Ethoxycarbonylphenyl)ureido]nicotinic acid methyl ester
4-[3-(6-Chloropyridin-3-yl)ureido]benzoic acid ethyl ester
4-[3-(4-Carboxymethylphenyl)ureido]benzoic acid ethyl ester
4-[3-(1H-Indol-5-yl)ureido]benzoic acid ethyl ester
1-(4-Fluorophenyl)-3-(4-morpholin-4-ylphenyl)urea
1-Benzothiazol-6-yl-3-(4-fluorophenyl)urea
1-(4-Fluorophenyl)-3-(4-imidazol-1-ylphenyl)urea
6-[3-(4-Fluorophenyl)ureido]nicotinic acid methyl ester
1-(6-Chlorobenzothiazol-2-yl)-3-(4-fluorophenyl)urea
1-(6-Fluorobenzothiazol-2-yl)-3-(4-fluorophenyl)urea
1-(4,6-Difluorobenzothiazol-2-yl)-3-(4-fluorophenyl)urea
1-(4-Fluorophenyl)-3-(6-methoxybenzothiazol-2-yl)urea
1-(4-Fluorophenyl)-3-[3-(2-methylpyrimidin-4-yl)phenyl]urea
3-[3-(4-Fluorophenyl)ureido]benzoic acid methyl ester
1-(4-Fluorophenyl)-3-(2-fluorophenyl)urea
3-[3-(4-Fluorophenyl)ureido]benzoic acid ethyl ester
1-(4-Fluoro-3-methylphenyl)-3-(4-fluorophenyl)urea
4-{3-[3-(2-Methylpyrimidin-4-yl)phenyl]ureido}benzoic acid ethyl ester
4-[3-(1-Oxoindan-5-yl)ureido]benzoic acid ethyl ester
4-[3-(6-Morpholin-4-ylpyridin-3-yl)ureido]benzoic acid ethyl ester
2-[3-(4-Ethoxycarbonylphenyl)ureido]thiazole-5-carboxylic acid methyl ester
4-[3-(3-Ethoxycarbonylphenyl)ureido]benzoic acid ethyl ester
4-[3-(4-Ethoxycarbonylphenyl)ureido]benzoic acid propyl ester
4-[3-(4-Ethoxycarbonylphenyl)ureido]benzoic acid pentyl ester
4-[3-(4-Ethoxycarbonylphenyl)ureido]benzoic acid isobutyl ester
4-[3-(4-Ethoxycarbonylphenyl)ureido]benzoic acid phenyl ester
4-{3-[4-(1,1,2,2-Tetrafluoroethoxy)phenyl]ureido}benzoic acid ethyl ester
4-[3-(3-Oxazol-5-ylphenyl)ureido]benzoic acid ethyl ester
4-[3-(4-Ethoxycarbonylmethylphenyl)ureido]benzoic acid ethyl ester
4-[3-(4-[1,2,3]Thiadiazol-4-ylphenyl)ureido]benzoic acid ethyl ester
4-[3-(4-Propionylphenyl)ureido]benzoic acid ethyl ester
4-[3-(4-Acetylphenyl)ureido]benzoic acid ethyl ester
4-[3-(4-Benzoylphenyl)ureido]benzoic acid ethyl ester
4-{3-[4-(4,5-Dihydrooxazol-2-yl)phenyl]ureido}benzoic acid ethyl ester
4-{3-[4-(2-Methylpyrimidin-4-yl)phenyl]ureido}benzoic acid ethyl ester
1-(4-Fluorophenyl)-3-(4-pyrrol-1-ylphenyl)urea
1-(4-Fluorophenyl)-3-(2-methylbenzothiazol-5-yl)urea
1-(4-Fluorophenyl)-3-(3-oxazol-5-ylphenyl)urea
1-(4-Fluorophenyl)-3-(4-propionylphenyl)urea
1-(4-Fluorophenyl)-3-[4-(2-methylpyrimidin-4-yl)phenyl]urea
4-[3-(4-Ethoxycarbonylphenyl)ureido]benzoic acid butyl ester
2-[3-(4-Ethoxycarbonylphenyl)ureido]-4-methylpyrimidine-5-carboxylic acid ethyl ester
4-[3-(4-Oxazol-5-yl phenyl)ureido]benzoic acid ethyl ester
2-Chloro-4-[3-(4-ethoxycarbonylphenyl)ureido]benzoic acid ethyl ester
4-[3-(4-Ethoxycarbonylphenyl)ureido]-2-methoxybenzoic acid ethyl ester
4-[3-(4-Ethoxycarbonylphenyl)ureido]-3-methoxybenzoic acid ethyl ester
6-[3-(4-Ethoxycarbonylphenyl)ureido]nicotinic acid ethyl ester
4-[3-(4-Fluorophenyl)ureido]-3-hydroxy benzoic acid ethyl ester
4-[3-(3-Acetylphenyl)ureido]benzoic acid ethyl ester
4-[3-(4-Butyrylphenyl)ureido]benzoic acid ethyl ester
4-{3-[4-(1H-Pyrazol-3-yl)phenyl]ureido}benzoic acid ethyl ester
4-[3-(4-Fluorophenyl)ureido]benzoic acid propyl ester
4-[3-(4-Fluorophenyl)ureido]benzoic acid pentyl ester
4-[3-(4-Fluorophenyl)ureido]benzoic acid isobutyl ester
4-[3-(4-Fluorophenyl)ureido]benzoic acid phenyl ester
{4-[3-(4-Fluorophenyl)ureido]phenyl}acetic acid ethyl ester
1-(4-Benzoylphenyl)-3-(4-fluorophenyl)urea
1-(4-Butyrylphenyl)-3-(4-fluorophenyl)urea
4-[3-(4-Fluorophenyl)ureido]benzoic acid butyl ester
2-Chloro-4-[3-(4-fluorophenyl)ureido]benzoic acid ethyl ester
1-[2-(3-Fluorophenyl)ethyl]-3-(4-isopropylphenyl)urea
1-[2-(2-Fluorophenyl)ethyl]-3-(4-isopropylphenyl)urea
1-[2-(3-Fluorophenyl)ethyl]-3-(4-trifluoromethylphenyl)urea
1-(4-Isopropylphenyl)-3-thiazol-2-ylurea
1-(4-Acetylphenyl)-3-(4-bromophenyl)urea
1-[2-(4-Chlorophenyl)ethyl]-3-[3-(2-methylpyrimidin-4-yl)phenyl]urea
1-(4-Chlorophenyl)-3-(3-pyrrol-1-ylphenyl)urea
1-(4-Chlorophenyl)-3-(4-pyrrol-1-ylphenyl)urea
1-(4-Chlorophenyl)-3-[3-(2-methylpyrimidin-4-yl)phenyl]urea
1-(4-Chlorophenyl)-3-[4-(3,4-dihydro-1H-isoquinolin-2-yl)-3-fluorophenyl]urea
1-(3,4-Dichlorophenyl)-3-(3-fluoro-4-piperidin-1-ylphenyl)urea
1-(4-Chlorophenyl)-3-[3-(6-pyrrolidin-1-ylpyridin-2-yl)phenyl]urea
1-(4-Azepan-1-yl-3-fluorophenyl)-3-(4-chlorophenyl)urea
1-(4-Chlorophenyl)-3-(3-fluoro-4-pyrrolidin-1-ylphenyl)urea
1-(3-Fluoro-4-piperidin-1-ylphenyl)-3-[4-(2-morpholin-4-ylethoxy)phenyl]urea
1-(3-Fluoro-4-piperidin-1-ylphenyl)-3-[4-(2-methoxyethoxy)phenyl]urea
1-(4-Chlorophenyl)-3-[3-(2-isopropylpyrimidin-4-yl)phenyl]urea
1-(4-Chlorophenyl)-3-(3-fluoro-4-[1,4]oxazepan-4-ylphenyl)urea
1-(3-Fluoro-4-piperidin-1-ylphenyl)-3-[4-(morpholine-4-carbonyl)phenyl]urea
1-(3-Fluoro-4-piperidin-1-ylphenyl)-3-(4-pyrrol-1-ylphenyl)urea
4-[3-(3-Fluoro-4-piperidin-1-ylphenyl)ureido]benzoic acid ethyl ester
1-(3-Fluoro-4-piperidin-1-ylphenyl)-3-[3-(2-methoxyethoxy)phenyl]urea
1-(3-Fluoro-4-piperidin-1-ylphenyl)-3-[3-(2-morpholin-4-ylethoxy)phenyl]urea
1-(4-Chlorophenyl)-3-(4-pyridin-3-ylphenyl)urea
1-(4-Chlorophenyl)-3-[3-(6-methylpyrimidin-4-yl)phenyl]urea
1-(4-Chlorophenyl)-3-[3-fluoro-4-(3-hydroxypiperidin-1-yl)phenyl]urea
1-(4-Chlorophenyl)-3-(4-pyridin-2-yl-phenyl)urea
1-(4-Chlorophenyl)-3-(4-pyridin-4-ylphenyl)urea
1-(4-Chlorophenyl)-3-[3-(2-piperidin-1-ylpyrimidin-4-yl)phenyl]urea
1-(3-Fluoro-4-piperidin-1-ylphenyl)-3-[4-(2-morpholin-4-ylethyl)phenyl]urea
1-(3-Fluoro-4-piperidin-1-ylphenyl)-3-(4-morpholin-4-ylmethylphenyl)urea
1-(2,3-Dihydrobenzofuran-6-yl)-3-(3-fluoro-4-piperidin-1-ylphenyl)urea
1-(3,5-Dimethoxyphenyl)-3-(3-fluoro-4-piperidin-1-ylphenyl)urea
1-(4-Chlorophenyl)-3-(3-pyrazol-1-ylphenyl)urea
1-(4-Chlorophenyl)-3-[3-(3,4,5,6-tetrahydro-2H-[1,2′]bipyridinyl-6′-yl)phenyl]urea
1-(4-Morpholin-4-ylmethylphenyl)-3-(4-pyrrol-1-ylphenyl)urea
1-(4-Morpholin-4-ylmethylphenyl)-3-(3-pyrrol-1-ylphenyl)urea
1-(4-Chlorophenyl)-3-[4-(4,4-difluoropiperidin-1-yl)-3-fluorophenyl]urea
1-(4-Butyrylphenyl)-3-[4-(morpholine-4-carbonyl)phenyl]urea
1-(1-Methyl-1H-indazol-5-yl)-3-(4-morpholin-4-ylmethylphenyl)urea
1-(4-Morpholin-4-ylmethylphenyl)-3-(4-pyrazol-1-ylphenyl)urea
1-(2,3-Dihydrobenzo[1,4]dioxin-6-yl)-3-(3-fluoro-4-piperidin-1-ylphenyl)urea
1-(3,5-Dichlorophenyl)-3-(3-fluoro-4-piperidin-1-ylphenyl)urea
1-(3-Chloro-4-fluorophenyl)-3-(3-fluoro-4-piperidin-1-ylphenyl)urea
1-(4-Ethylphenyl)-3-(3-fluoro-4-piperidin-1-ylphenyl)urea
1-[2-(4-Chlorophenyl)ethyl]-3-[3-(2-methyl-2H-pyrazol-3-yl)phenyl]urea
1-(4-Chlorophenyl)-3-[3-fluoro-4-(4-hydroxypiperidin-1-yl)phenyl]urea
1-(4-Chlorophenyl)-3-[3-fluoro-4-(3-methylpiperidin-1-yl)phenyl]urea
1-Benzo[1,3]dioxol-5-yl-3-(3-fluoro-4-piperidin-1-ylphenyl)urea
1-(4-Chlorophenyl)-3-[3-fluoro-4-(2-methylpiperidin-1-yl)phenyl]urea
1-(3-Fluoro-4-piperidin-1-ylphenyl)-3-(4-methoxyphenyl)urea
1-(4-Chloro-2-hydroxyphenyl)-3-(3-fluoro-4-piperidin-1-ylphenyl)urea
1-(3-Fluoro-4-piperidin-1-ylphenyl)-3-[3-(2-methylpyrimidin-4-yl)phenyl]urea
1-(4-Chlorophenyl)-3-[3-fluoro-4-(4-trifluoromethylpiperidin-1-yl)phenyl]urea
1-(4-Chlorophenyl)-3-[3-fluoro-4-(4-methylpiperidin-1-yl)phenyl]urea
1-(3-Fluoro-4-piperidin-1-ylphenyl)-3-(4-phenoxyphenyl)urea
1-(3-Fluoro-4-piperidin-1-ylphenyl)-3-(3-phenoxyphenyl)urea
1-(4-Fluorophenyl)-3-(3-fluoro-4-piperidin-1-ylphenyl)urea
1-(3-Fluoro-4-piperidin-1-ylphenyl)-3-(3-methoxyphenyl)urea
1-(4-Cyanophenyl)-3-(3-fluoro-4-piperidin-1-ylphenyl)urea
1-(4-Chlorophenyl)-3-(4-morpholin-4-ylmethylphenyl)urea
1-(4-Chloro-3-trifluoromethylphenyl)-3-(3-fluoro-4-piperidin-1-ylphenyl)urea
1-(3-Fluoro-4-piperidin-1-ylphenyl)-3-(4-trifluoromethylphenyl)urea
1-(3-Chlorophenyl)-3-(3-fluoro-4-piperidin-1-ylphenyl)urea
1-(4-Chlorophenyl)-3-(3,4,5,6-tetrahydro-2H-[1,2′]bipyridinyl-5′-yl)urea
1-(4-Chlorophenyl)-3-(3-dimethylaminophenyl)urea
1-(4-Chlorophenyl)-3-(3-fluoro-4-morpholin-4-ylphenyl)urea
1-[2-(4-Chlorophenyl)ethyl]-3-(3-pyrrol-1-ylphenyl)urea
1-(3,5-Dichlorophenyl)-3-(3,4,5,6-tetrahydro-2H-[1,2′]bipyridinyl-5′-yl)urea
1-(3-Chlorophenyl)-3-(3,4,5,6-tetrahydro-2H-[1,2]bipyridinyl-5′-yl)urea
1-(3,5-Bis-trifluoromethylphenyl)-3-(3,4,5,6-tetrahydro-2H-[1,2′]bipyridinyl-5′-yl)urea
1-(4-Acetylphenyl)-3-[4-(morpholine-4-carbonyl)phenyl]urea
1-(4-Acetylphenyl)-3-[3-(6-methoxypyridin-2-yl)phenyl]urea
1-(4-Acetylphenyl)-3-(4-morpholin-4-ylmethylphenyl)urea
1-(4-Chlorophenyl)-3-(3-fluoro-4-piperidin-1-ylphenyl)urea
1-(3-Chloro-4-morpholin-4-ylphenyl)-3-(4-chlorophenyl)urea
1-(4-Chlorophenyl)-3-(4-piperidin-1-ylphenyl)urea
1-(4-Acetylphenyl)-3-(3,4,5,6-tetrahydro-2H-[1,2′]bipyridinyl-5′-yl)urea
1-(4-Butyrylphenyl)-3-(4-piperidin-1-ylphenyl)urea
1-[2-(4-Chlorophenyl)ethyl]-3-(4-morpholin-4-ylmethylphenyl)urea
1-(4-Chlorophenyl)-3-(1-methyl-1H-indazol-5-yl)urea
1-(4-Chlorophenyl)-3-[3-(2-pyrrolidin-1-ylpyrimidin-4-yl)phenyl]urea
1-(4-Chlorophenyl)-3-(4-pyrazol-1-ylphenyl)urea
1-[2-(4-Chlorophenyl)ethyl]-3-[4-(morpholine-4-carbonyl)phenyl]urea

or a pharmaceutically acceptable salt thereof, in combination with a pharmaceutically acceptable carrier.

Preferably the composition is comprised of a pharmaceutically acceptable carrier and a non-toxic therapeutically effective amount of a compound of formula (I), or a pharmaceutically acceptable salt thereof.

Moreover, within this preferred embodiment, the invention encompasses a pharmaceutical composition for the treatment of disease by modulating the CB-1 receptor, resulting in the suppression of appetite, comprising a pharmaceutically acceptable carrier and a non-toxic therapeutically effective amount of a compound of formula (I) or a pharmaceutically acceptable salt thereof.

The pharmaceutical compositions of the present invention, or administered by the methods of the present invention, comprise a compound of formula (I) or a pharmaceutically acceptable salt thereof, as an active ingredient, a pharmaceutically acceptable carrier and optionally other therapeutic ingredients or adjuvants. The compositions include compositions suitable for oral, rectal, topical, and parenteral (including subcutaneous, intramuscular, and intravenous) administration, although the most suitable route in any given case will depend on the particular host, and nature and severity of the conditions for which the active ingredient is being administered. The pharmaceutical compositions may be conveniently presented in unit dosage form and prepared by any of the methods well known in the art of pharmacy.

In practice, the compounds of formula (I), or pharmaceutically acceptable salts thereof, can be combined as the active ingredient in intimate admixture with a pharmaceutical carrier according to conventional pharmaceutical compounding techniques. The carrier may take a wide variety of forms depending on the form of preparation desired for administration, e.g. oral or parenteral (including intravenous). Thus, the pharmaceutical compositions can be presented as discrete units suitable for oral administration such as capsules, cachets or tablets each containing a predetermined amount of the active ingredient. Further, the compositions can be presented as a powder, as granules, as a solution, as a suspension in an aqueous liquid, as a non-aqueous liquid, as an oil-in-water emulsion, or as a water-in-oil liquid emulsion. In addition to the common dosage forms set out above, the compound of formula (I), or a pharmaceutically acceptable salt thereof, may also be administered by controlled release means and/or delivery devices. The compositions may be prepared by any of the methods of pharmacy. In general, such methods include a step of bringing into association the active ingredient with the carrier that constitutes one or more necessary ingredients. In general, the compositions are prepared by uniformly and intimately admixing the active ingredient with liquid carriers or finely divided solid carriers or both. The product can then be conveniently shaped into the desired presentation.

Thus, the pharmaceutical compositions may include a pharmaceutically acceptable carrier and a compound of formula (I), or a pharmaceutically acceptable salt thereof. The compounds of formula (I), or pharmaceutically acceptable salts thereof, can also be included in pharmaceutical compositions in combination with one or more other therapeutically active compounds.

The pharmaceutical carrier employed can be, for example, a solid, liquid, or gas. Examples of solid carriers include lactose, terra alba, sucrose, talc, gelatin, agar, pectin, acacia, magnesium stearate, and stearic acid. Examples of liquid carriers are sugar syrup, peanut oil, olive oil, and water. Examples of gaseous carriers include carbon dioxide and nitrogen.

In preparing the compositions for oral dosage form, any convenient pharmaceutical media may be employed. For example, water, glycols, oils, alcohols, flavoring agents, preservatives, coloring agents, and the like may be used to form oral liquid preparations such as suspensions, elixirs and solutions; while carriers such as starches, sugars, microcrystalline cellulose, diluents, granulating agents, lubricants, binders, disintegrating agents, and the like may be used to form oral solid preparations such as powders, capsules and tablets. Because of their ease of administration, tablets and capsules are the preferred oral dosage units whereby solid pharmaceutical carriers are employed. Optionally, tablets may be coated by standard aqueous or nonaqueous techniques.

A tablet containing the composition of the invention may be prepared by compression or molding, optionally with one or more accessory ingredients or adjuvants. Compressed tablets may be prepared by compressing, in a suitable machine, the active ingredient in a free-flowing form such as powder or granules, optionally mixed with a binder, lubricant, inert diluent, surface active or dispersing agent. Molded tablets may be made by molding in a suitable machine, a mixture of the powdered compound moistened with an inert liquid diluent. Each tablet preferably contains from about 0.05 mg to about 5 g of the active ingredient and each cachet or capsule preferably containing from about 0.05 mg to about 5 g of the active ingredient. For example, a formulation intended for the oral administration to humans may contain from about 0.5 mg to about 5 g of active agent, compounded with an appropriate and convenient amount of carrier material which may vary from about 5 to about 95 percent of the total composition. Unit dosage forms will generally contain between from about 1 mg to about 2 g of the active ingredient, typically 25 mg, 50 mg, 100 mg, 200 mg, 300 mg, 400 mg, 500 mg, 600 mg, 800 mg, or 1000 mg.

Pharmaceutical compositions of the present invention suitable for parenteral administration may be prepared as solutions or suspensions of the active compounds in water. A suitable surfactant can be included such as, for example, hydroxypropylcellulose. Dispersions can also be prepared in glycerol, liquid polyethylene glycols, and mixtures thereof in oils. Further, a preservative can be included to prevent the detrimental growth of microorganisms.

Pharmaceutical compositions of the present invention suitable for injectable use include sterile aqueous solutions or dispersions. Furthermore, the compositions can be in the form of sterile powders for the extemporaneous preparation of such sterile injectable solutions or dispersions. In all cases, the final injectable form must be sterile and must be effectively fluid for easy syringability. The pharmaceutical compositions must be stable under the conditions of manufacture and storage; thus, preferably should be preserved against the contaminating action of microorganisms such as bacteria and fungi. The carrier can be a solvent or dispersion medium containing, for example, water, ethanol, polyol (e.g. glycerol, propylene glycol and liquid polyethylene glycol), vegetable oils, and suitable mixtures thereof.

Pharmaceutical compositions of the present invention can be in a form suitable for topical use such as, for example, an aerosol, cream, ointment, lotion, dusting powder, or the like. Further, the compositions can be in a form suitable for use in transdermal devices. These formulations may be prepared, using a compound of formula (I), or a pharmaceutically acceptable salt thereof, via conventional processing methods. As an example, a cream or ointment is prepared by admixing hydrophilic material and water, together with about 5 wt % to about 10 wt % of the compound, to produce a cream or ointment having a desired consistency.

Pharmaceutical compositions of this invention can be in a form suitable for rectal administration wherein the carrier is a solid. It is preferable that the mixture forms unit dose suppositories. Suitable carriers include cocoa butter and other materials commonly used in the art. The suppositories may be conveniently formed by first admixing the composition with the softened or melted carrier(s) followed by chilling and shaping in molds.

In addition to the aforementioned carrier ingredients, the pharmaceutical formulations described above may include, as appropriate, one or more additional carrier ingredients such as diluents, buffers, flavoring agents, binders, surface-active agents, thickeners, lubricants, preservatives (including anti-oxidants) and the like. Furthermore, other adjuvants can be included to render the formulation isotonic with the blood of the intended recipient. Compositions containing a compound of formula (I), or pharmaceutically acceptable salts thereof, may also be prepared in powder or liquid concentrate form.

The compositions of the present invention or used in the present invention are effective to suppress appetite, to prophylactically prevent overweight, to assist in regulating food intake, to assist as a diet aid, and to treat obesity. Generally, dosage levels on the order of from about 0.01 mg/kg to about 150 mg/kg of body weight per day are useful in the treatment of the above-indicated conditions, or alternatively about 0.5 mg to about 7 g per patient per day. For example, obesity may be effectively treated by the administration of from about 0.01 to 50 mg of the compound per kilogram of body weight per day, or alternatively about 0.5 mg to about 3.5 g per patient per day.

It is understood, however, that the specific dose level for any particular patient will depend upon a variety of factors including the age, body weight, general health, sex, diet, time of administration, route of administration, rate of excretion, drug combination and the severity of the particular disease undergoing therapy.

All publications, including, but not limited to, patents and patent application cited in this specification, are herein incorporated by reference as if each individual publication were specifically and individually indicated to be incorporated by reference herein as fully set forth.

The invention will now be described by reference to the following examples which are for illustrative purposes and are not to be construed as a limitation of the scope of the present invention.

### Materials and Methods:

Column chromatography was carried out on SiO₂ (40-63 mesh). LCMS data were obtained using a Waters Symmetry 3.5μ C₁₈ column (2.1×30.0 mm, flow rate=0.8 mL/min) eluting with a (5% MeCN in H₂O)-MeCN solution containing 0.1% HCO₂H over 6 min and UV detection at 220 nm. Gradient information: 0.0-1.2 min: 100% (5% MeCN in H₂O); 1.2-3.8 min: Ramp up to 10% (5% MeCN in H₂O)-90% MeCN; 3.8-4.4 min: Hold at 10% (5% MeCN in H₂O)-90% MeCN; 4.4-5.5 min: Ramp up to 100% MeCN; 5.5-6.0 min: Return to 100% (5% MeCN in H₂O). The mass spectra were obtained employing an electrospray ionisation source in the positive (ES⁺) ion mode. Prep HPLC purification was carried out using a Lunar 10μ ODS2 (250×21.2 mm; Flow rate=20 mL/min) eluting with solvent A (0.05% TFA, 10% MeCN, 90% water) and solvent B (0.05% TFA, 90% MeCN, 10% water) and UV detection at 215 nm. Gradient information: 0.0-0.2 min: 90% A, 10% B; 0.2-10.0 min: Ramp up to 10% A, 90% B; 10.0-15.0 min: 10% A, 90% B; 15.0-16.0 min: Return to 90% A, 10% B.
Abbreviations and acronyms: MeCN: Acetonitrile; DME: Dimethylether; DIPEA: N,N-Diisopropylethylamine; DMF: N,N-Dimethylformamide; Et₂O: Diethyl ether; EtOAc: Ethyl acetate; EtOH: Ethanol; MeOH: Methanol; PS: Polymer supported; rt: room temperature RT: Retention time; THF: Tetrahydrofuran; TFA: Trifluoroacetic acid; Et₃N: Triethylamine.

### Preparation 1

### 2-(2-Fluoro-4-nitrophenyl)-1,2,3,4-tetrahydroisoquinoline

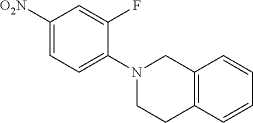

To a solution of 3,4-difluoronitrobenzene (5 g, 31.4 mmol) in EtOAc (50 mL) was added 1,2,3,4-tetrahydroisoquinoline (4.60 g, 34.5 mmol) and Et₃N (4.79 mL, 34.5 mmol) and refluxed for 3 h. The reaction mixture was cooled to rt and washed with sodium carbonate (20 mL), dried (MgSO₄) and concentrated in vacuo to give the title compound: δ_{H} (CD₃OD): 2.97 (2H, t), 3.68 (2H, t), 4.57 (2H, s), 7.19-7.23 (5H, m), 8.01-8.05 (2H, m).

### Preparation 2

### 4-(3,4-Dihydro-1H-isoquinolin-2-yl)-3-fluorophenylamine

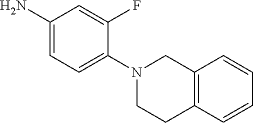

To a solution of 2-(2-fluoro-4-nitrophenyl)-1,2,3,4-tetrahydroisoquinoline (2.5 g, 9.18 mmol) in ethanol (220 mL) and THF (15 mL) was added palladium (10%) on activated carbon (973 mg, 0.92 mmol) and stirred under an atmosphere of hydrogen at rt for 18 h. The reaction mixture was filtered through celite and concentrated in vacuo to yield the title compound: RT=2.49 min; m/z (ES⁺)=243.1 [M+H]⁺.

### Preparation 3

### 1-(2-Fluoro-4-nitrophenyl)piperidine

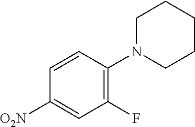

Prepared using the method outlined for Preparation 1 using piperidine as the amine: δ_{H} (DMSO): 1.58-1.67 (6H, m), 3.26-3.29 (4H, m), 7.12-7.16 (1H, m), 7.94-7.80 (2H, m).

### Preparation 4

### 3-Fluoro-4-piperidin-1-ylphenylamine

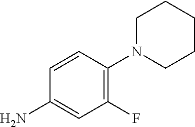

Prepared from reduction of 1-(2-fluoro-4-nitrophenyl)piperidine using the method outlined in Preparation 2 to give the title compound: δ_{H} (DMSO): 1.43-1.49 (2H, m), 1.57-1.62 (4H, m), 2.75-2.77 (4H, t), 4.92 (2H, s), 6.27-6.34 (2H, m), 6.72-6.77 (1H, m).

### Preparation 5

### 2-Bromo-6-pyrrolidin-1-ylpyridine

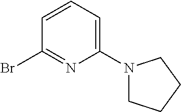

A mixture of 2,6-dibromopyridine (5.00 g, 21.10 mmol) and pyrrolidine (10 mL) was stirred for 20 h. The reaction mixture was partitioned between CH₂Cl₂ and saturated NaHCO₃ (aq), the organic phase was dried (MgSO₄) and the solvent was removed under vacuum. The resulting solid was recrystallised (MeOH) to give the title compound: RT=3.84 min; m/z (ES⁺)=227.04 [M+H]⁺.

### Preparation 6

### 2-(3-Nitrophenyl)-6-pyrrolidin-1-yl pyridine

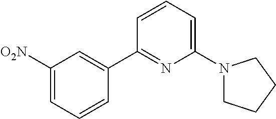

Argon was bubbled through a mixture of 3-nitrophenylboronic acid (1.22 g, 7.30 mmol), 2-bromo-6-pyrrolidin-1-yl pyridine (1.50 g, 6.64 mmol) and NaHCO₃ (1.67 g, 19.91 mmol) in DME (60 mL) and water (25 mL) for 15 min. Pd(Ph₃)₄ (0.64 g, 0.553 mmol) was added and the reaction refluxed under argon for 4 h. The solvent was removed under vacuum and the resulting residue purified by flash chromatography (SiO₂, eluting with 20:80, 40:60 then 60:40 CH₂Cl₂, i-hexane) to give the title compound: RT=3.45 min; m/z (ES⁺)=270.16 [M+H]⁺.

### Preparation 7

### 1-(2-Fluoro-4-nitrophenyl)azepane

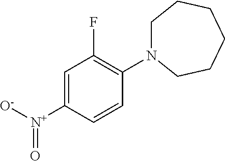

A solution of 3,4-difluoronitrobenzene (0.20 g, 1.26 mmol), homopiperidine (0.14 g, 1.38 mmol) and Et₃N (0.14 g, 1.38 mmol) in EtOAc (2 mL) was heated at 80° C. for 20 h. Homopiperidine (0.14 g, 1.38 mmol) was added and the reaction heated at 80° C. for 4 h. The solid was purified using an SPE cartridges (SCX, eluting with MeOH) to give the title compound: RT=4.17 min; m/z (ES⁺)=239.04 [M+H]⁺.

### Preparation 8

### 1-(2-Fluoro-4-nitrophenyl)pyrrolidine

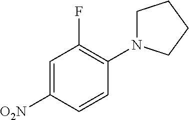

A solution of 3,4-difluoronitrobenzene (0.20 g, 1.26 mmol), pyrrolidine (98 mg, 1.38 mmol) and Et₃N (0.14 g, 1.38 mmol) in EtOAc (2 mL) was heated at 80° C. for 20 h. Pyrrolidine (98 mg, 1.38 mmol) was added and the reaction heated at 80° C. for 4 h. The solid was purified using an SPE cartridge (SCX, eluting with MeOH) to give the title compound: RT=3.84 min; m/z (ES⁺)=211.01 [M+H]⁺.

### Example 1

### 2-[3-(4-Fluorophenyl)ureido]-4-methylthiazole-5-carboxylic acid ethyl ester

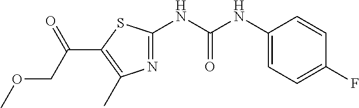

A mixture of ethyl (2-amino-4-methylthiazole)-5-carboxylate (60 mg, 0.32 mmol) and 4-fluorophenyl isocyanate (48 mg, 0.35 mmol) in toluene (5 mL) was stirred for 20 h at 20° C. The precipitate was collected by filtration to give the title compound: RT=3.86 min; m/z (ES⁺)=324.1 [M+H]⁺.
Addition of ethyl (2-amino-4-methylthiazole)-5-carboxylate to the appropriate phenyl isocyanates, as outlined in EXAMPLE 1, was also used to synthesise EXAMPLES 2 to 5 listed in TABLE 1 below.

TABLE 1

| | | | RT | m/z |
|---|---|---|---|---|
| Ex | Structure | Name | (min) | (ES⁺) |
| 2 | 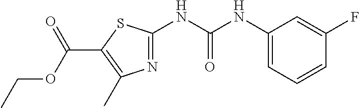 | 2-[3-(3-Fluorophenyl)ureido]- 4-methylthiazole-5-carboxylic acid ethyl ester | 3.94 | 324.1 [M + H]⁺ |
| 3 | 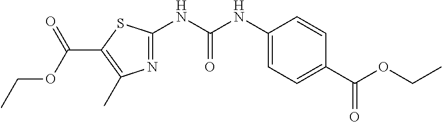 | 2-[3-(4- Ethoxycarbonylphenyl) ureido]-4- methylthiazole-5-carboxylic acid ethyl ester | 3.88 | 378.1 [M + H]⁺ |
| 4 | 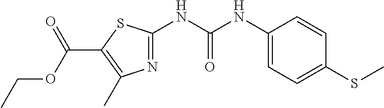 | 4-Methyl-2-[3-(4- methylsulfanylphenyl) ureido]thiazole- 5-carboxylic acid ethyl ester | 3.95 | 352.1 [M + H]⁺ |
| 5 | 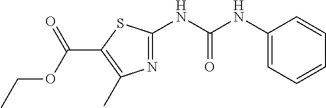 | 4-Methyl-2-[3- phenylureido] thiazole-5-carboxylic acid ethyl ester | 3.79 | 306.1 [M + H]⁺ |

### Example 6

### 1-(4-Acetylphenyl)-3-benzo[b]thiophen-2-ylurea

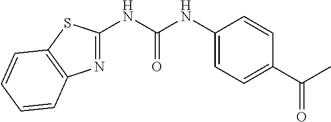

To a solution of benzothiophene-2-carbonyl chloride (0.64 g, 3.26 mmol) in THF (10 mL) at 0° C. was added a solution of sodium azide (0.25 g, 3.85 mmol) in water (2 mL) over 10 min dropwise. The reaction mixture was extracted with Et₂O (20 mL), CH₂Cl₂ (2×20 mL) and EtOAc (2×10 mL). The organic extracts were combined, dried (MgSO₄) and the solvent removed under vacuum to give a solid, which was taken up in toluene (30 mL) and refluxed under argon for 90 min. The mixture was cooled to 20° C., 4-aminoacetophenone (0.44 g, 3.25 mmol) was added and the reaction was stirred for 18 h. The precipitate was collected by filtration and purified by flash chromatography (SiO₂, eluting with 5:95, 10:90 then 20:80 EtOAc, CH₂Cl₂) to give the title compound: RT=3.73 min; m/z (ES⁺)=311.1 [M+H]⁺.

### Example 7

### 1-Benzo[b]thiophen-2-yl-3-(4-methanesulfonylphenyl)urea

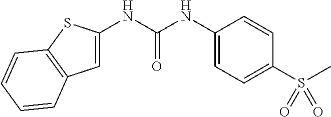

The Curtius rearrangement to give benzothiophene-2-isocyanate followed by addition of the appropriate aniline, as outlined in EXAMPLE 6, was used to synthesise the title compound: RT=3.55 min; m/z (ES⁺)=347.1 [M+H]⁺.
The compounds in TABLE 2 are commercially available, however they can be prepared from the appropriate acid chlorides and anilines using the method outlined in EXAMPLE 6.

TABLE 2

| | | | | RT | m/z |
|---|---|---|---|---|---|
| Ex | Structure | Name | Source | (min) | (ES⁺) |
| 8 | 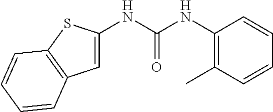 | 1-Benzo[b] thiophen-2-yl-3- (2-methylphenyl) urea | Maybridge | 3.84 | 283.0 [M + H]⁺ |
| 9 | 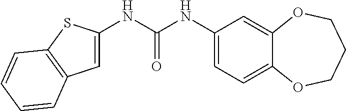 | 1-Benzo[b] thiophen-2-yl-3- (3,4-dihydro-2H- benzo[b] [1,4]dioxepin-7- yl)urea | Maybridge | 4.02 | 340.9 [M + H]⁺ |
| 10 | 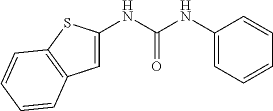 | 1-Benzo[b] thiophen-2-yl-3- phenylurea | Maybridge | 3.89 | 269.0 [M + H]⁺ |
| 11 | 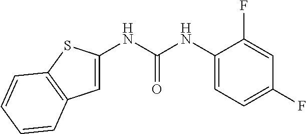 | 1-Benzo[b] thiophen-2-yl-3- (2,4- difluorophenyl) urea | Maybridge | 3.97 | 304.9 [M + H]⁺ |
| 12 | 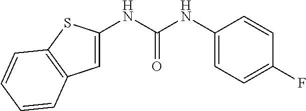 | 1-Benzo[b] thiophen-2-yl-3- (4-fluorophenyl) urea | Maybridge | 3.87 | 287.0 [M + H]⁺ |
| 13 | 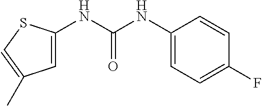 | 1-(4- Fluorophenyl)- 3-(4- methylthiophen- 2-yl)urea | Maybridge | 3.62 | 251.0 [M + H]⁺ |
| 14 | 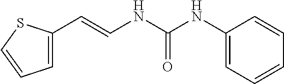 | 1-Phenyl-3-(2- thiophen-2- ylvinyl)urea | Maybridge | 3.62 | 245.1 [M + H]⁺ |
| 15 | 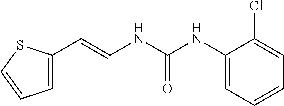 | 1-(2- Chlorophenyl)- 3-(2- thiophen-2- ylvinyl)urea | Maybridge | 3.92 | 278.9 [M + H]⁺ |

### Example 16

### 4-[3-(4-Fluoro-2-methylphenyl)ureido]benzoic acid ethyl ester

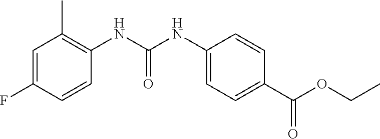

To a solution of 4-fluoro-2-methylaniline (34 mg, 0.27 mmol) in toluene (0.5 mL) was added ethyl 4-isocyanatobenzoate (50 mg, 0.26 mmol) in toluene (0.5 mL). The reaction mixture was stirred for 18 h and the resulting precipitate was collected by filtration to give the title compound: RT=3.70 min; m/z (ES⁺)=317.3 [M+H]⁺.
Addition of the appropriate anilines to ethyl 4-isocyanatobenzoate, as outlined in EXAMPLE 16, was also used to synthesise EXAMPLES 17 to 25 listed in TABLE 3 below.

TABLE 3

| | | | RT | m/z |
|---|---|---|---|---|
| Ex | Structure | Name | (min) | (ES⁺) |
| 17 | 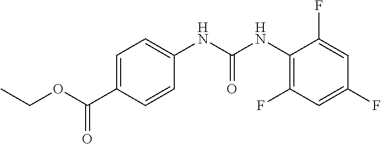 | 4-[3-(2,4,6-Trifluorophenyl) ureido]benzoic acid ethyl ester | 3.56 | 339.2 [M + H]⁺ |
| 18 | 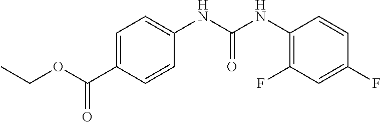 | 4-[3-(2,4-Difluorophenyl) ureido]benzoic acid ethyl ester | 3.65 | 321.2 [M + H]⁺ |
| 19 | 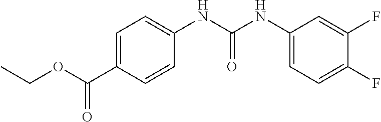 | 4-[3-(3,4-Difluorophenyl) ureido]benzoic acid ethyl ester | 3.99 | 321.2 [M + H]⁺ |
| 20 | 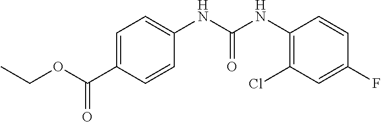 | 4-[3-(2-Chloro-4- fluorophenyl) ureido]benzoic acid ethyl ester | 3.89 | 337.2 [M + H]⁺ |
| 21 | 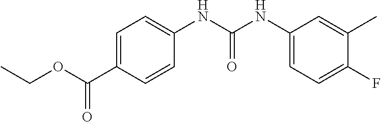 | 4-[3-(4-Fluoro-3- methylphenyl) ureido]benzoic acid ethyl ester | 3.79 | 317.3 [M + H]⁺ |
| 22 | 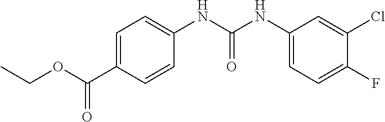 | 4-[3-(3-Chloro-4- fluorophenyl) ureido]benzoic acid ethyl ester | 3.82 | 337.2 [M + H]⁺ |
| 23 | 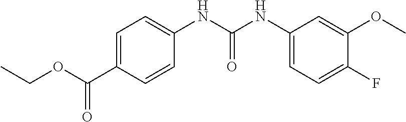 | 4-[3-(4-Fluoro-3- methoxyphenyl) ureido]benzoic acid ethyl ester | 3.84 | 333.3 [M + H]⁺ |
| 24 | 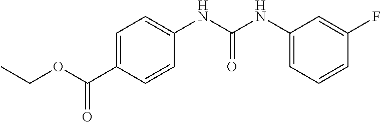 | 4-[3-(3-Fluorophenyl)ureido] benzoic acid ethyl ester | 3.76 | 303.2 [M + H]⁺ |
| 25 | 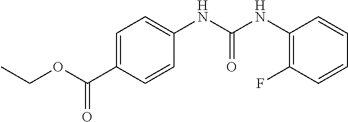 | 4-[3-(2-Fluorophenyl)ureido] benzoic acid ethyl ester | 3.62 | 303.2 [M + H]⁺ |

Addition of appropriate amines to 4-fluorophenyl isocyanate, as outlined in EXAMPLE 16, was also used to synthesise EXAMPLES 26 to 34 listed in TABLE 4 below.

TABLE 4

| | | | RT | m/z |
|---|---|---|---|---|
| Ex | Structure | Name | (min) | (ES⁺) |
| 26 | 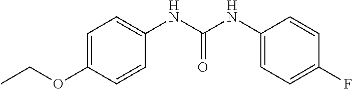 | 1-(4-Ethoxyphenyl)-3-(4- fluorophenyl)urea | 3.56 | 275.2 [M + H]⁺ |
| 27 | 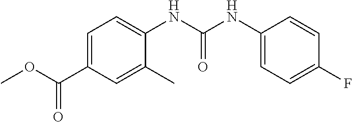 | 4-[3-(4-Fluorophenyl)ureido]- 3-methylbenzoic acid methyl ester | 3.66 | 303.2 [M + H]⁺ |
| 28 | 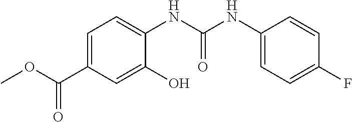 | 4-[3-(4-Fluorophenyl)ureido]- 3-hydroxybenzoic acid methyl ester | 3.65 | 305.2 [M + H]⁺ |
| 29 | 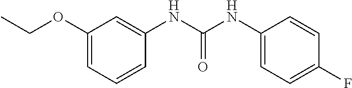 | 1-(3-Ethoxyphenyl)-3-(4- fluorophenyl)urea | 3.60 | 275.2 [M + H]⁺ |
| 30 | 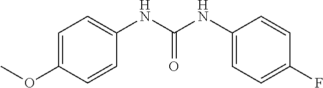 | 1-(4-Fluorophenyl)-3-(4- methoxyphenyl)urea | 3.50 | 261.2 [M + H]⁺ |
| 31 | 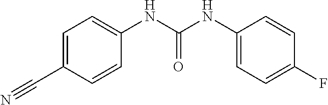 | 1-(4-Cyanophenyl)-3-(4- fluorophenyl)urea | 3.60 | 256.2 [M + H]⁺ |
| 32 | 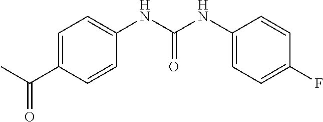 | 1-(4-Acetylphenyl)-3-(4- fluorophenyl)urea | 3.33 | 273.2 [M + H]⁺ |
| 33 | 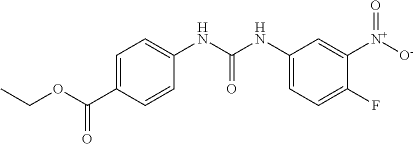 | 4-[3-(4-Fluoro-3- nitrophenyl)ureido] benzoic acid ethyl ester | 3.77 | 348.2 [M + H]⁺ |
| 34 | 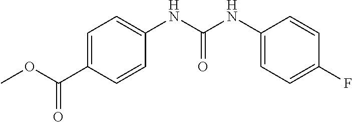 | 4-[3-(4-Fluorophenyl)ureido] benzoic acid methyl ester | 3.52 | 289.2 [M + H]⁺ |

Addition of appropriate amities to the appropriate phenyl isocyanate, as outlined in EXAMPLE 16, was also used to synthesise EXAMPLES 35 to 39 listed in TABLE 5 below.

TABLE 5

| | | | RT | m/z |
|---|---|---|---|---|
| Ex | Structure | Name | (min) | (ES⁺) |
| 35 | 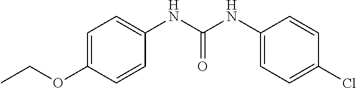 | 1-(4-Chlorophenyl)-3-(4- ethoxyphenyl)urea | 3.77 | 291.1 [M + H]⁺ |
| 36 | 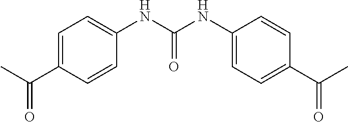 | 1,3-Bis(4-acetylphenyl)urea | 3.29 | 297.1 [M + H]⁺ |
| 37 | 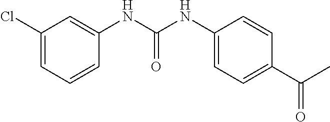 | 1-(4-Acetylphenyl)-3-(3- chlorophenyl)urea | 3.59 | 289.1 [M + H]⁺ |
| 38 | 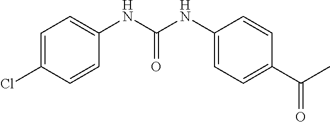 | 1-(4-Acetylphenyl)-3-(4- chlorophenyl)urea | 3.60 | 577.3 [2M + H]⁺ |
| 39 | 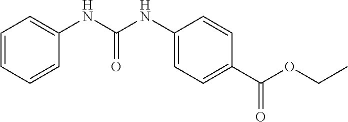 | 4-(3-Phenylureido)benzoic acid ethyl ester | 3.66 | 285.2 [M + H]⁺ |

### Example 40

### 1-(2-Thiophen-2-ylethyl)-3-(4-methylphenyl)urea

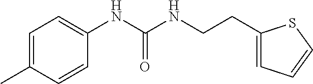

A solution of 2-thiophen-2-ylethylamine (30 mg, 0.24 mmol) in toluene (3 mL) was added to p-tolyl isocyanate (47 mg, 0.35 mmol) and shaken for 18 h. The resulting precipitate was filtered and washed with toluene to give the title compound: RT=3.70 min; m/z (ES⁺)=261.0 [M+H]⁺.
Addition of 2-thiophen-2-ylethylamine to the appropriate isocyanates, as outlined in EXAMPLE 40, was also used to synthesise EXAMPLES 41 to 53 listed in TABLE 6 below.

TABLE 6

| | | | RT | m/z |
|---|---|---|---|---|
| Ex | Structure | Name | (min) | (ES⁺) |
| 41 | 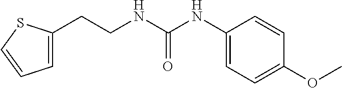 | 1-(4-Methoxyphenyl)-3-(2- thiophen-2-ylethyl)urea | 3.40 | 277.0 [M + H]⁺ |
| 42 | 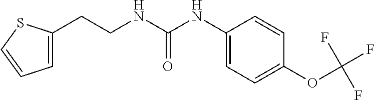 | 1-(2-Thiophen-2-ylethyl)-3-(4- trifluoromethoxyphenyl)urea | 3.85 | 331.1 [M + H]⁺ |
| 43 | 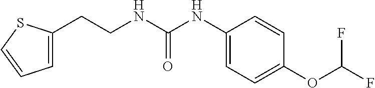 | 1-(4-Difluoromethoxy-phenyl)- 3-(2-thiophen-2-ylethyl)urea | 3.96 | 313.0 [M + H]⁺ |
| 44 | 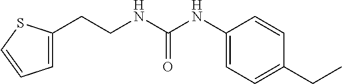 | 1-(4-Ethylphenyl)-3-(2- thiophen-2-ylethyl)urea | 3.75 | 275.1 [M + H]⁺ |
| 45 | 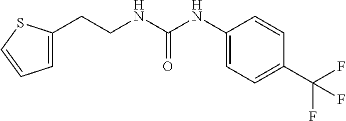 | 1-(2-Thiophen-2-ylethyl)-3-(4- trifluoromethylphenyl)urea | 3.83 | 315.1 [M + H]⁺ |
| 46 | 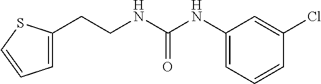 | 1-(3-Chlorophenyl)-3-(2- thiophen-2-ylethyl)urea | 3.83 | 281.1 [M + H]⁺ |
| 47 | 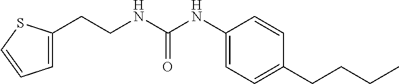 | 1-(4-Butylphenyl)-3-(2- thiophen-2-ylethyl)urea | 4.06 | 303.1 [M + H]⁺ |
| 48 | 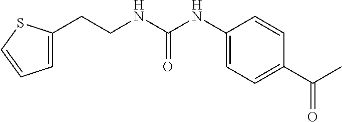 | 1-(4-Acetylphenyl)-3-(2- thiophen-2-ylethyl)urea | 3.38 | 289.1 [M + H]⁺ |
| 49 | 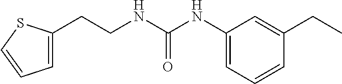 | 1-(3-Ethylphenyl)-3-(2- thiophen-2-ylethyl)urea | 3.83 | 275.1 [M + H]⁺ |
| 50 | 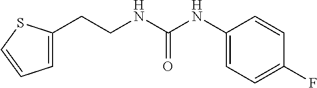 | 1-(4-Fluorophenyl)-3-(2- thiophen-2-ylethyl)urea | 3.57 | 265.1 [M + H]⁺ |
| 51 | 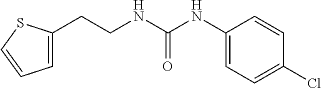 | 1-(4-Chlorophenyl)-3-(2- thiophen-2-ylethyl)urea | 3.71 | 281.1 [M + H]⁺ |
| 52 | 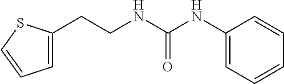 | 1-Phenyl-3-(2-thiophen-2- ylethyl)urea | 3.50 | 247.1 [M + H]⁺ |
| 53 | 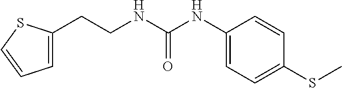 | 1-(4-Methylsulfanylphenyl)-3- (2-thiophen-2-ylethyl)urea | 3.70 | 293.1 [M + H]⁺ |

EXAMPLE 54 in TABLE 7 is commercially available, however it can be prepared using the method outlined in EXAMPLE 40.

TABLE 7

| | | | | RT | m/z |
|---|---|---|---|---|---|
| Ex | Structure | Name | Source | (min) | (ES⁺) |
| 54 | 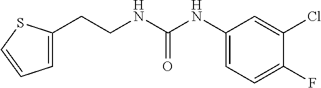 | 1-(3-Chloro-4- fluoro-phenyl)-3- (2-thiophen-2- ylethyl)urea | Tripos | 3.63 | 299.0 [M + H]⁺ |

EXAMPLE 55 in TABLE 8 can be prepared from the addition of 2-thiophen-2-ylethylamine to the appropriate isocyanate using the method outlined in EXAMPLE 40.

TABLE 8

| | | | RT | m/z |
|---|---|---|---|---|
| Ex | Structure | Name | (min) | (ES⁺) |
| 55 | 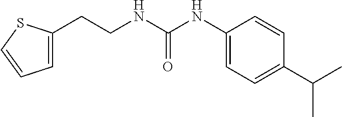 | 1-(4-Isopropylphenyl)-3-(2- thiophen-2-ylethyl)-urea | 3.78 | [M + H]⁺ |

### Example 56

### 4-(3-Benzothiazol-6-ylureido)benzoic acid ethyl ester

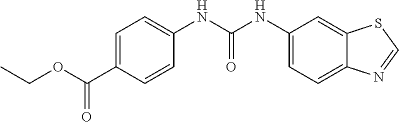

A solution of ethyl 4-isocyanatobenzoate (29 mg, 0.15 mmol) in DMF (1.7 mL) was added to 6-aminobenzothiazole (30 mg, 0.20 mmol) and shaken for 18 h. MP-isocyanate (360 mg, 0.58 mmol, 4.58 mmol/g, 3.9 eq) was added to the mixture and shaken for 20 h. The resin was removed by filtration and the solvent was removed under vacuum to give the title compound: RT=3.71 min; m/z (ES⁺)=341.9 [M+H]⁺.
Addition of the appropriate amines to ethyl 4-isocyanatobenzoate or 4-fluorophenyl isocyanate, as outlined in EXAMPLE 56, was also used to synthesise EXAMPLES 57 to 130 listed in TABLE 9 below.

TABLE 9

| | | | RT | m/z |
|---|---|---|---|---|
| Ex | Structure | Name | (min) | (ES⁺) |
| 57 | 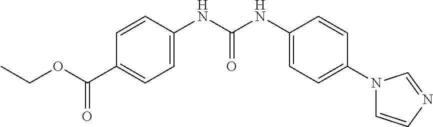 | 4-[3-(4-Imidazol-1- ylphenyl)ureido]benzoic acid ethyl ester | 2.90 | 351.2 [M + H]⁺ |
| 58 | 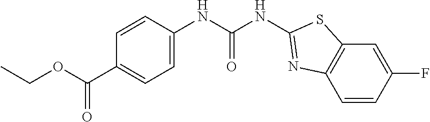 | 4-[3-(6- Fluorobenzothiazol- 2-yl)ureido]benzoic acid ethyl ester | 3.95 | 359.9 [M + H]⁺ |
| 59 | 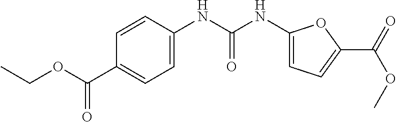 | 5-[3-(4- Ethoxycarbonylphenyl) ureido]furan-2- carboxylic acid methyl ester | 3.72 | 333.0 [M + H]⁺ |
| 60 | 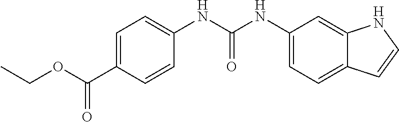 | 4-[3-(1H-Indol-6-yl) ureido]benzoic acid ethyl ester | 3.74 | 324.3 [M + H]⁺ |
| 61 | 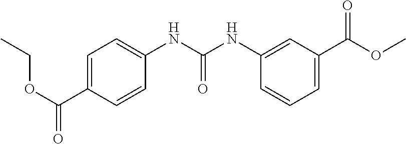 | 4-[3-(3- Methoxycarbonylphenyl) ureido]benzoic acid ethyl ester | 3.68 | 343.3 [M + H]⁺ |
| 62 | 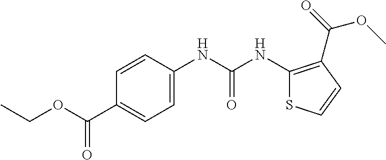 | 2-[3-(4- Ethoxycarbonylphenyl) ureido]thiophene-3- carboxylic acid methyl ester | 4.05 | 348.9 [M + H]⁺ |
| 63 | 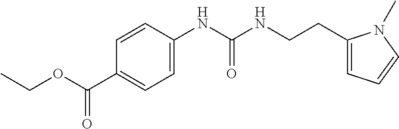 | 4-{3-[2-(1-Methyl-1H- pyrrol-2-yl)ethyl] ureido}benzoic acid ethyl ester | 3.64 | 316.3 [M + H]⁺ |
| 64 | 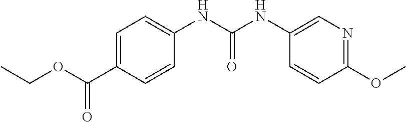 | 4-[3-(6-Methoxypyridin-3- yl)ureido]benzoic acid ethyl ester | 3.44 | 316.3 [M + H]⁺ |
| 65 | 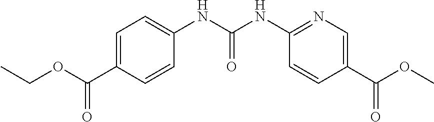 | 6-[3-(4- Ethoxycarbonylphenyl) ureido]nicotinic acid methyl ester | 4.06 | 344.0 [M + H]⁺ |
| 66 | 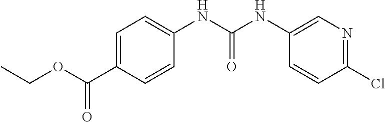 | 4-[3-(6-Chloropyridin-3- yl)ureido]benzoic acid ethyl ester | 3.40 | 320.2 [M + H]⁺ |
| 67 | 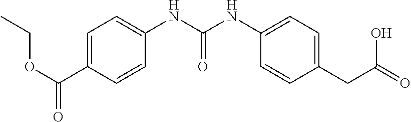 | 4-[3-(4- Carboxymethylphenyl) ureido]benzoic acid ethyl ester | 3.15 | 343.3 [M + H]⁺ |
| 68 | 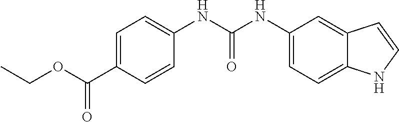 | 4-[3-(1H-Indol-5-yl) ureido]benzoic acid ethyl ester | 3.69 | 324.3 [M + H]⁺ |
| 69 | 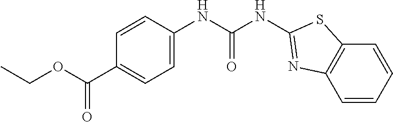 | 4-(3-Benzothiazol-2- ylureido)benzoic acid ethyl ester | 3.94 | 341.9 [M + H]⁺ |
| 70 | 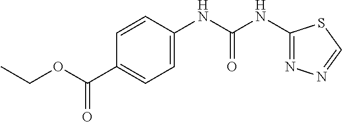 | 4-(3-[1,3,4]Thiadiazol-2- ylureido)benzoic acid ethyl ester | 3.19 | 293.2 [M + H]⁺ |
| 71 | 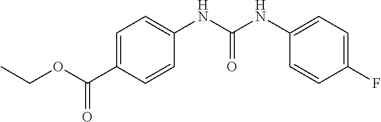 | 4-[3-(4-Fluorophenyl) ureido]benzoic acid ethyl ester | 3.67 | 303.3 [M + H]⁺ |
| 72 | 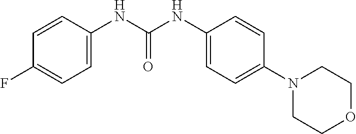 | 1-(4-Fluorophenyl)-3-(4- morpholin-4-ylphenyl)urea | 3.00 | 316.3 [M + H]⁺ |
| 73 | 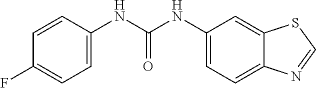 | 1-Benzothiazol-6-yl-3-(4- fluorophenyl)urea | 3.48 | 288.2 [M + H]⁺ |
| 74 | 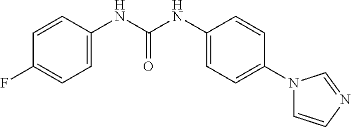 | 1-(4-Fluorophenyl)-3-(4- imidazol-1-ylphenyl)urea | 2.61 | 296.9 [M + H]⁺ |
| 75 | 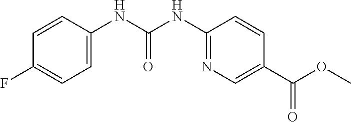 | 6-[3-(4-Fluorophenyl) ureido]nicotinic acid methyl ester | 3.50 | 290.2 [M + H]⁺ |
| 76 | 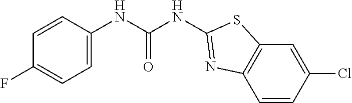 | 1-(6-Chlorobenzothiazol-2- yl)-3-(4-fluorophenyl)urea | 4.12 | 322.2 [M + H]⁺ |
| 77 | 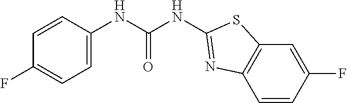 | 1-(6-Fluorobenzothiazol-2- yl)-3-(4-fluorophenyl)urea | 4.07 | 305.9 [M + H]⁺ |
| 78 | 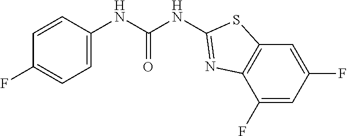 | 1-(4,6- Difluorobenzothiazol- 2-yl)- 3-(4-fluorophenyl)urea | 3.94 | 323.9 [M + H]⁺ |
| 79 | 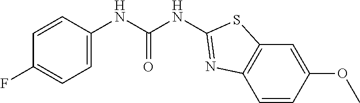 | 1-(4-Fluorophenyl)-3-(6- methoxybenzothiazol-2- yl)urea | 3.59 | 318.2 [M + H]⁺ |
| 80 | 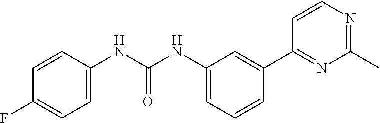 | 1-(4-Fluorophenyl)-3-[3-(2- methylpyrimidin-4-yl) phenyl]urea | 3.32 | 323.2 [M + H]⁺ |
| 81 | 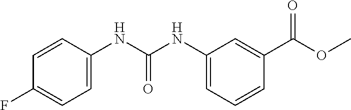 | 3-[3-(4-Fluorophenyl) ureido]benzoic acid methyl ester | 3.50 | 289.3 [M + H]⁺ |
| 82 | 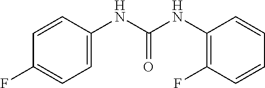 | 1-(4-Fluorophenyl)-3-(2- fluorophenyl)urea | 3.52 | 249.2 [M + H]⁺ |
| 83 | 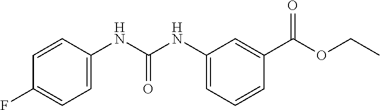 | 3-[3-(4-Fluorophenyl) ureido]benzoic acid ethyl ester | 3.54 | 303.3 [M + H]⁺ |
| 84 | 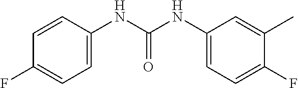 | 1-(4-Fluoro-3- methylphenyl)- 3-(4-fluoro phenyl)urea | 3.61 | 263.2 [M + H]⁺ |
| 85 | 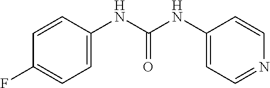 | 1-(4-Fluorophenyl)-3- pyridin-4-ylurea | 2.31 | 232.2 [M + H]⁺ |
| 86 | 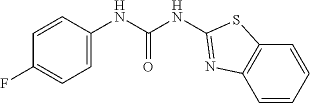 | 1-Benzothiazol-2-yl-3-(4- fluorophenyl)urea | 3.61 | 288.2 [M + H]⁺ |
| 87 | 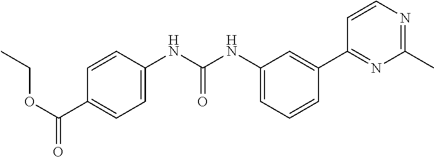 | 4-{3-[3-(2- Methylpyrimidin- 4-yl)phenyl] ureido}benzoic acid ethyl ester | 3.64 | 377.1 [M + H]⁺ |
| 88 | 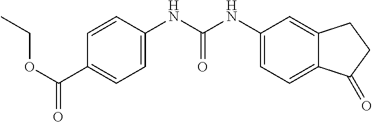 | 4-[3-(1-Oxoindan-5-yl) ureido]benzoic acid ethyl ester | 3.56 | 339.1 [M + H]⁺ |
| 89 | 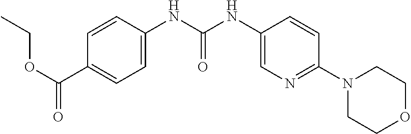 | 4-[3-(6-Morpholin-4-yl- pyridin-3-yl)ureido] benzoic acid ethyl ester | 2.84 | 371.1 [M + H]⁺ |
| 90 | 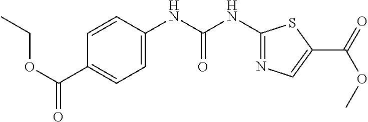 | 2-[3-(4- Ethoxycarbonylphenyl) ureido]thiazole-5- carboxylic acid methyl ester | 3.81 | 350.1 [M + H]⁺ |
| 91 | 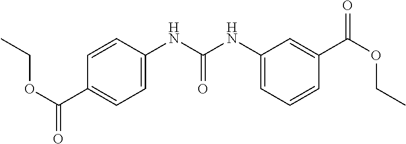 | 4-[3-(3- Ethoxycarbonylphenyl) ureido]benzoic acid ethyl ester | 3.94 | 357.1 [M + H]⁺ |
| 92 | 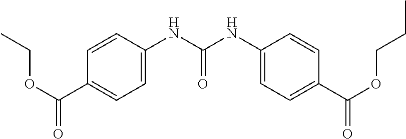 | 4-[3-(4- Ethoxycarbonylphenyl) ureido]benzoic acid propyl ester | 4.07 | 371.1 [M + H]⁺ |
| 93 | 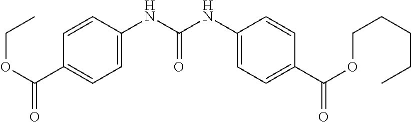 | 4-[3-(4- Ethoxycarbonylphenyl) ureido]benzoic acid pentyl ester | 4.57 | 399.1 [M + H]⁺ |
| 94 | 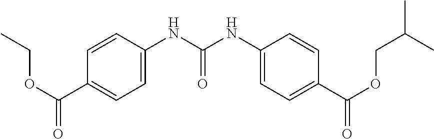 | 4-[3-(4- Ethoxycarbonylphenyl) ureido]benzoic acid isobutyl ester | 4.14 | 385.1 [M + H]⁺ |
| 95 | 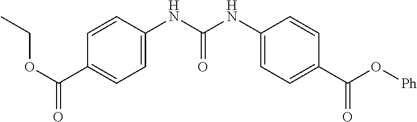 | 4-[3-(4- Ethoxycarbonylphenyl) ureido]benzoic acid phenyl ester | 4.09 | 405.1 [M + H]⁺ |
| 96 | 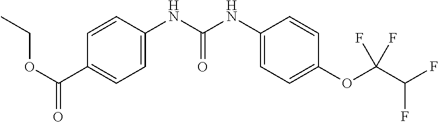 | 4-{3-[4-(1,1,2,2- Tetrafluoroethoxy) phenyl] ureido}benzoic acid ethyl ester | 3.94 | 401.0 [M + H]⁺ |
| 97 | 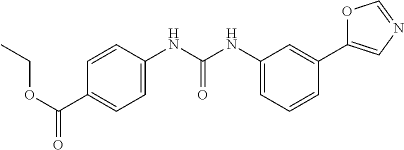 | 4-[3-(3-Oxazol-5- ylphenyl) ureido]benzoic acid ethyl ester | 3.90 | 352.1 [M + H]⁺ |
| 98 | 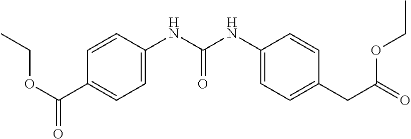 | 4-[3-(4- Ethoxycarbonylmethylphenyl) ureido] benzoic acid ethyl ester | 3.81 | 371.1 [M + H]⁺ |
| 99 | 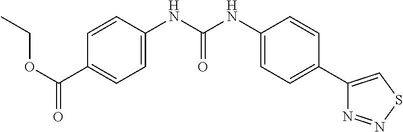 | 4-[3-(4-[1,2,3]Thiadiazol-4- ylphenyl)ureido]benzoic acid ethyl ester | 3.79 | 369.0 [M + H]⁺ |
| 100 | 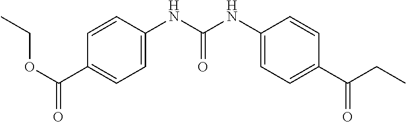 | 4-[3-(4-Propionylphenyl) ureido]benzoic acid ethyl ester | 3.74 | 341.1 [M + H]⁺ |
| 101 | 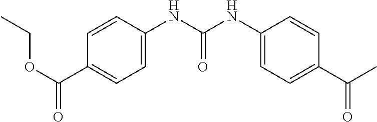 | 4-[3-(4-Acetylphenyl) ureido]benzoic acid ethyl ester | 3.77 | 327.1 [M + H]⁺ |
| 102 | 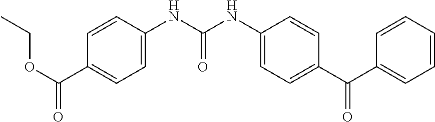 | 4-[3-(4-Benzoylphenyl) ureido]benzoic acid ethyl ester | 3.99 | 389.1 [M + H]⁺ |
| 103 | 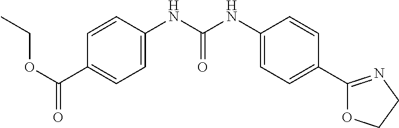 | 4-{3-[4-(4,5- Dihydrooxazol- 2-yl)phenyl] ureido}benzoic acid ethyl ester | 2.87 | 354.0 [M + H]⁺ |
| 104 | 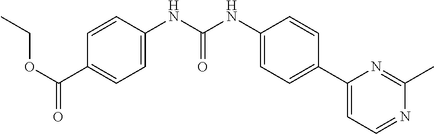 | 4-{3-[4-(2- Methylpyrimidin- 4-yl)phenyl] ureido}benzoic acid ethyl ester | 3.61 | 377.0 [M + H]⁺ |
| 105 | 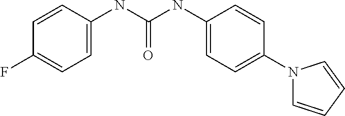 | 1-(4-Fluorophenyl)-3-(4- pyrrol-1-ylphenyl)urea | 4.01 | 296.1 [M + H]⁺ |
| 106 | 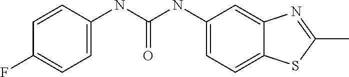 | 1-(4-Fluorophenyl)-3-(2- methylbenzothiazol-5- yl)urea | 3.45 | 302.0 [M + H]⁺ |
| 107 | 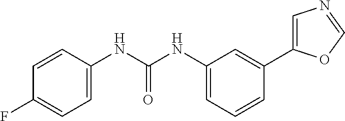 | 1-(4-Fluorophenyl)-3-(3- oxazol-5-ylphenyl)urea | 3.56 | 298.1 [M + H]⁺ |
| 108 | 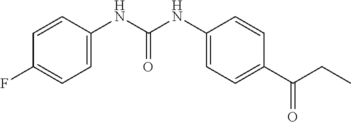 | 1-(4-Fluorophenyl)-3-(4- propionylphenyl)urea | 3.81 | 287.1 [M + H]⁺ |
| 109 | 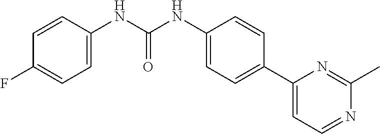 | 1-(4-Fluorophenyl)-3-[4-(2- methylpyrimidin-4- yl)phenyl]urea | 3.39 | 323.0 [M + H]⁺ |
| 110 | 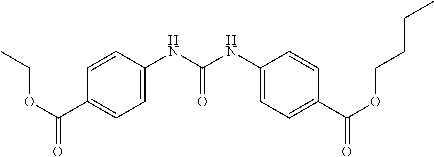 | 4-[3-(4- Ethoxycarbonylphenyl) ureido]benzoic acid butyl ester | 4.16 | 385.1 [M + H]⁺ |
| 111 | 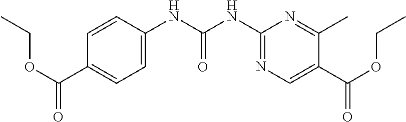 | 2-[3-(4- Ethoxycarbonylphenyl) ureido]-4- methylpyrimidine- 5-carboxylic acid ethyl ester | 3.87 | 373.1 [M + H]⁺ |
| 112 | 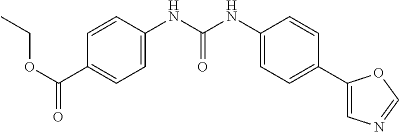 | 4-[3-(4-Oxazol-5- ylphenyl) ureido]benzoic acid ethyl ester | 3.90 | 352.1 [M + H]⁺ |
| 113 | 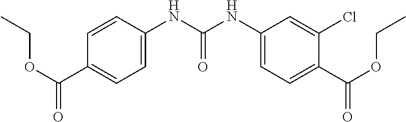 | 2-Chloro-4-[3-(4- ethoxycarbonylphenyl) ureido] benzoic acid ethyl ester | 4.04 | 391.1 [M + H]⁺ |
| 114 | 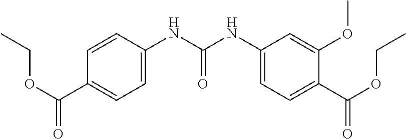 | 4-[3-(4- Ethoxycarbonylphenyl) ureido]-2- methoxybenzoic acid ethyl ester | 3.74 | 387.1 [M + H]⁺ |
| 115 | 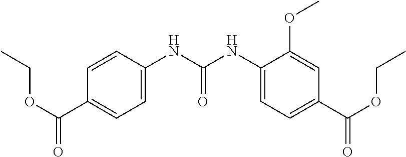 | 4-[3-(4- Ethoxycarbonylphenyl) ureido]-3- methoxybenzoic acid ethyl ester | 4.04 | 387.1 [M + H]⁺ |
| 116 | 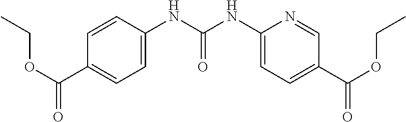 | 6-[3-(4- Ethoxycarbonylphenyl) ureido]nicotinic acid ethyl ester | 4.09 | 358.1 [M + H]⁺ |
| 117 | 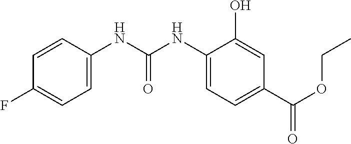 | 4-[3-(4-Fluorophenyl) ureido]-3-hydroxy benzoic acid ethyl ester | 3.69 | 319.1 [M + H]⁺ |
| 118 | 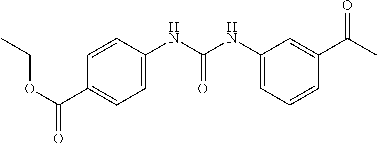 | 4-[3-(3-Acetylphenyl) ureido]benzoic acid ethyl ester | 3.70 | 327.0 [M + H]⁺ |
| 119 | 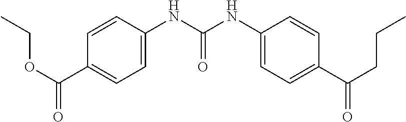 | 4-[3-(4-Butyrylphenyl) ureido]benzoic acid ethyl ester | 4.20 | 355.0 [M + H]⁺ |
| 120 | 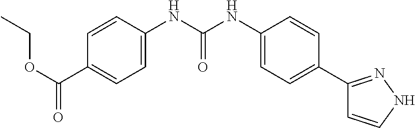 | 4-{3-[4-(1H-Pyrazol-3- yl)phenyl]ureido}benzoic acid ethyl ester | 3.74 | 351.0 [M + H]⁺ |
| 121 | 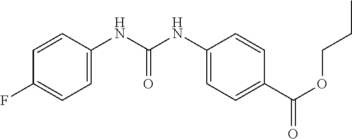 | 4-[3-(4-Fluorophenyl) ureido]benzoic acid propyl ester | 4.05 | 317.0 [M + H]⁺ |
| 122 | 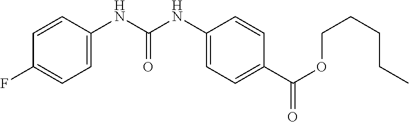 | 4-[3-(4-Fluorophenyl) ureido]benzoic acid pentyl ester | 4.49 | 345.0 [M + H]⁺ |
| 123 | 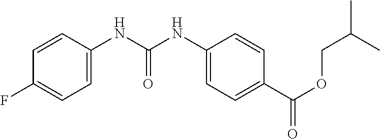 | 4-[3-(4-Fluorophenyl) ureido]benzoic acid isobutyl ester | 4.20 | 331.0 [M + H]⁺ |
| 124 | 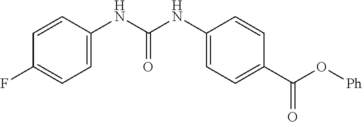 | 4-[3-(4-Fluorophenyl) ureido]benzoic acid phenyl ester | 4.17 | 351.0 [M + H]⁺ |
| 125 | 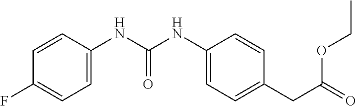 | {4-[3-(4-Fluorophenyl) ureido]phenyl}acetic acid ethyl ester | 4.04 | 317.0 [M + H]⁺ |
| 126 | 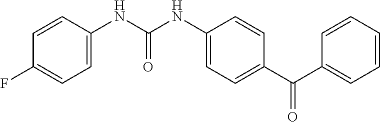 | 1-(4-Benzoylphenyl)-3-(4- fluorophenyl)urea | 3.99 | 335.0 [M + H]⁺ |
| 127 | 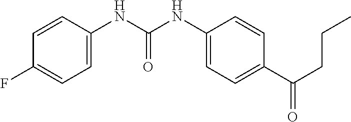 | 1-(4-Butyrylphenyl)-3-(4- fluorophenyl)urea | 4.20 | 301.0 [M + H]⁺ |
| 128 | 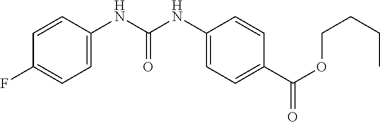 | 4-[3-(4-Fluorophenyl) ureido]benzoic acid butyl ester | 4.20 | 331.0 [M + H]⁺ |
| 129 | 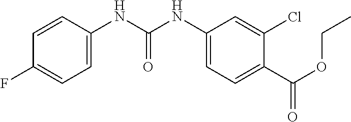 | 2-Chloro-4-[3-(4- fluorophenyl)ureido] benzoic acid ethyl ester | 4.09 | 336.9 [M + H]⁺ |

EXAMPLES 130 to 153 in TABLE 10 are commercially available, however can be prepared using the method outlined in EXAMPLE 56.

TABLE 10

| | | | | RT | m/z |
|---|---|---|---|---|---|
| Ex | Structure | Name | Source | (min) | (ES⁺) |
| 130 | 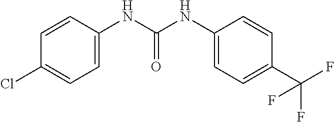 | 1-(4- Chlorophenyl)- 3-(4- trifluoromethylphenyl) urea | Tim Tec | 4.14 | 314.9 [M + H]⁺ |
| 131 | 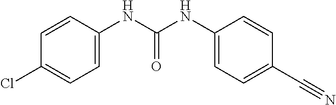 | 1-(4- Chlorophenyl)- 3-(4- cyanophenyl)urea | Chembridge | 3.81 | 271.9 [M + H]⁺ |
| 132 | 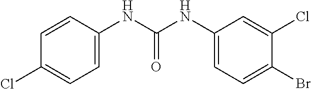 | 1-(4-Bromo-3- chlorophenyl)-3- (4-chlorophenyl) urea | Exploratory Library | 4.26 | 360.8 [M + H]⁺ |
| 133 | 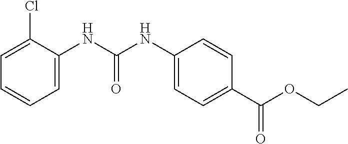 | 4-[3-(2- Chlorophenyl) ureido] benzoic acid ethyl ester | SALOR | 3.99 | 318.9 [M + H]⁺ |
| 134 | 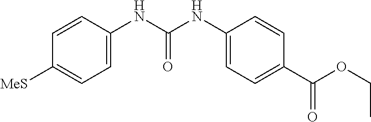 | 4-[3-(4- Methylsulfanylphenyl) ureido)benzoic acid ethyl ester | SALOR | 3.87 | 331.0 [M + H]⁺ |
| 135 | 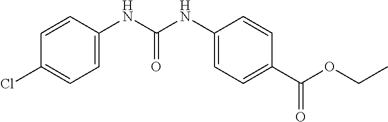 | 4-[3-(4- Chlorophenyl) ureido] benzoic acid ethyl ester | SALOR | 3.85 | 639.3 [2M + H]⁺ |
| 136 | 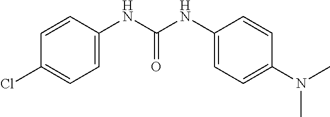 | 1-(4- Chlorophenyl)- 3-(4- dimethylaminophenyl) urea | SALOR | 2.81 | 289.9 [M + H]⁺ |
| 137 | 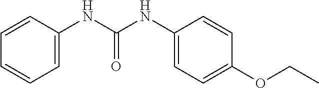 | 1-Phenyl-3-(4- ethoxyphenyl) urea | SALOR | 3.60 | 257.2 [M + H]⁺ |
| 138 | 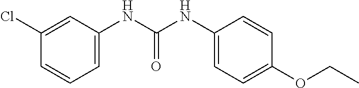 | 1-(3- Chlorophenyl)- 3-(4- ethoxyphenyl) urea | Chembridge | 3.73 | 291.2 [M + H]⁺ |
| 139 | 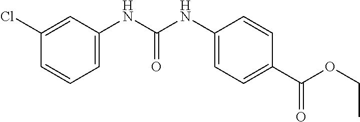 | 4-[3-(3- Chlorophenyl)ureido] benzoic acid ethyl ester | ChemDiv | 4.33 | 319.0 [M + H]⁺ |
| 140 | 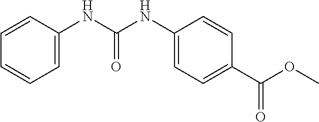 | 4-(3- Phenylureido) benzoic acid methyl ester | Tim Tec | 3.48 | 271.2 [M + H]⁺ |
| 141 | 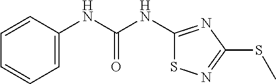 | 1-(3- Methylsulfanyl [1,2,4] thiadiazol-5-yl)- 3-phenylurea | SPECS | 3.59 | 266.9 [M + H]⁺ |
| 142 | 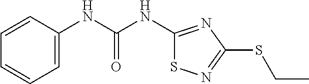 | 1-(3- Ethylsulfanyl [1,2,4] thiadiazol-5-yl)- 3-phenylurea | SPECS | 3.63 | 281.1 [M + H]⁺ |
| 143 | 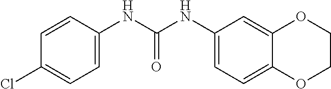 | 1-(4- Chlorophenyl)- 3-(2,3- dihydrobenzo [1,4]dioxan-6- yl)urea | Chembridge | 3.92 | 304.9 [M + H]⁺ |
| 144 | 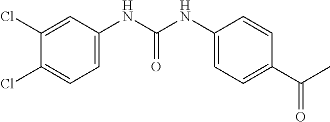 | 1-(4- Acetylphenyl)- 3-(3,4- dichlorophenyl) urea | Sigma | 3.94 | 322.9 [M + H]⁺ |
| 145 | 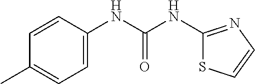 | 1-Thiazol-2-yl-3- (4-methylphenyl) urea | Chembridge | 3.39 | 233.9 [M + H]⁺ |
| 146 | 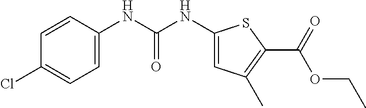 | 5-[3-(4- Chlorophenyl) ureido]-3- methylthiophene- 2-carboxylic acid ethyl ester | Chembridge | 3.94 | 338.9 [M + H]⁺ |
| 147 | 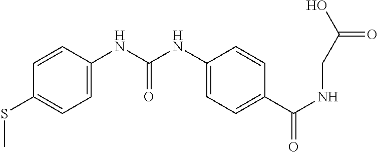 | {4-[3-(4- Methylsulfanylphenyl) ureido] benzoylamino} acetic acid | SALOR | 3.42 | 360.0 [M + H]⁺ |
| 148 | 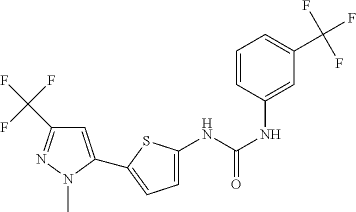 | 1-[5-(2-Methyl- 5- trifluoromethyl- 2H- pyrazol-3-yl) thiophen-2-yl]-3- (3- trifluoromethylphenyl) urea | Maybridge | 3.83 | 434.9 [M + H]⁺ |
| 149 | 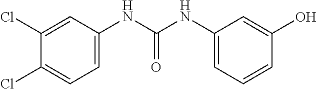 | 1-(3,4- Dichlorophenyl)- 3-(3- hydroxyphenyl) urea | SALOR | 3.58 | 297.1 [M + H]⁺ |
| 150 | 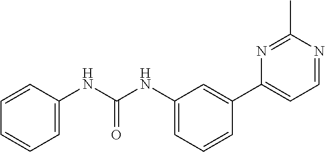 | 1-[3-(2- Methylpyrimidin- 4-yl) phenyl]-3- phenylurea | Maybridge | 3.42 | 304.9 [M + H]⁺ |
| 151 | 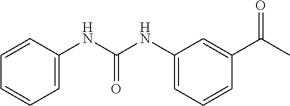 | 1-(3- Acetylphenyl)- 3- phenylurea | SALOR | 3.33 | 255.2 [M + H]⁺ |
| 152 | 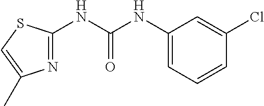 | 1-(3- Chlorophenyl)- 3-(4- methylthiazol-2- yl)urea | Chembridge | 3.63 | 268.1 [M + H]⁺ |
| 153 | 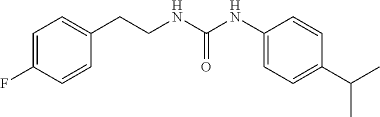 | 1-[2-(4- Fluorophenyl) ethyl]-3-(4- isopropylphenyl) urea | Chembridge | 3.87 | 301.2 [M + H]⁺ |

EXAMPLES 154 and 155 in TABLE 11, which have been previously reported, can be prepared from the appropriate aniline and isocyanate using the method outlined in EXAMPLE 56.

TABLE 11

| | | | RT | m/z |
|---|---|---|---|---|
| Ex | Structure | Name | (min) | (ES⁺) |
| 154 | 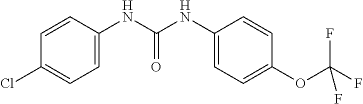 | 1-(4-Chlorophenyl)-3-(4- trifluoromethoxyphenyl)urea | 4.11 | 330.9 [M + H]⁺ |
| 155 | 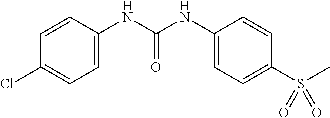 | 1-(4-Chlorophenyl)-3-(4- methanesulfonylphenyl)urea | 3.77 | 324.9 [M + H]⁺ |

EXAMPLES 156 to 161 in TABLE 12 can be prepared from the appropriate aniline and isocyanate using the method outlined in EXAMPLE 56.

TABLE 12

| | | | RT | m/z |
|---|---|---|---|---|
| Ex | Structure | Name | (min) | (ES⁺) |
| 156 | 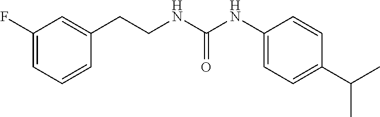 | 1-[2-(3-Fluorophenyl)ethyl]- 3-(4-isopropylphenyl)urea | 3.87 | 301.2 [M + H]⁺ |
| 157 | 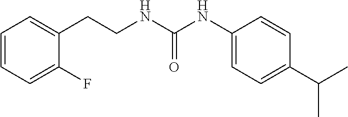 | 1-[2-(2-Fluorophenyl)ethyl]- 3-(4-isopropylphenyl)urea | 3.87 | 301.2 [M + H]⁺ |
| 158 | 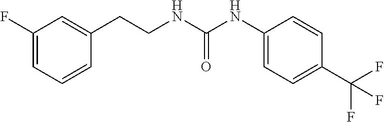 | 1-[2-(3-Fluorophenyl)ethyl]- 3-(4-trifluoromethylphenyl) urea | 3.83 | 327.2 [M + H]⁺ |
| 159 | 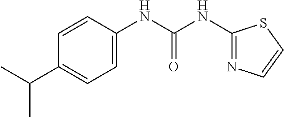 | 1-(4-Isopropylphenyl)-3- thiazol-2-ylurea | 3.62 | 262.2 [M + H]⁺ |
| 160 | 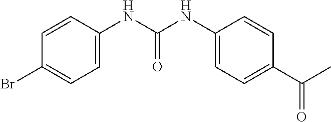 | 1-(4-Acetylphenyl)-3-(4- bromophenyl)urea | 3.97 | 332.9 [M + H]⁺ |
| 161 | 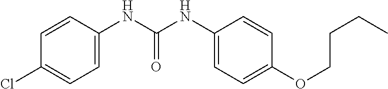 | 1-(4-Butoxyphenyl)-3-(4- chlorophenyl)urea | 4.39 | 319.0 [M + H]⁺ |

### Example 162

### 1-[2-(4-Chlorophenyl)ethyl]-3-[3-(2-methylpyrimidin-4-yl)phenyl]urea

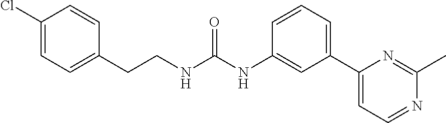

To a solution of triphosgene (400 mg, 1.35 mmol) in CH₂Cl₂ (50 mL) at 0° C. was added DIPEA (0.4 mL, 4.0 mmol) followed by 3-(2-methylpyrimidin-4-yl)phenylamine (740 mg, 4.0 mmol) in portions. After addition, the ice bath was removed, DIPEA (0.4 mL, 4.0 mmol) and 2-(4-chlorophenyl)ethylamine (622 mg, 4.0 mmol) were added. The reaction mixture was stirred for 18 h. CH₂Cl₂ (50 mL) was added and the organics were washed with water (20 mL), 1M NaOH solution (20 mL) and brine (20 mL) before being dried (MgSO₄) and removing the solvent in vacuo. The resulting powder was recrystallised from methanol to give the title compound: δ_{H}(DMSO): 2.67 (3H, s), 2.76 (2H, t), 3.35 (2H, m), 6.12 (1H, m), 7.28 (21-1, d), 7.36-7.40 (3H, m), 7.61 (1H, d), 7.67 (1H, d), 7.75 (1H, d), 8.19 (1H, s), 8.72-8.73 (2H, m); RT=3.57 min; m/z (ES⁺)=367.19 [M+H]⁺.

### Example 163

### 1-(4-Chlorophenyl)-3-(3-pyrrol-1-ylphenyl)urea

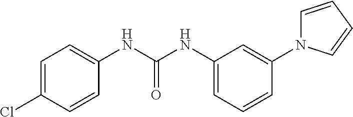

To a solution of 4-chlorophenylisocyanate (100 mg, 0.65 mmol) in CH₂Cl₂ (7 mL) was added 3-(1H-pyrrol-1-yl)aniline (103 mg, 0.65 mmol) at rt. The reaction mixture was stirred at rt for 4 h and the resulting solid collected by filtration. Trituration with Et₂O, followed by filtration gave the title compound: δ_{H} (DMSO): 6.27 (2H, t), 7.15-7.18 (1H, m), 7.24-7.27 (3H, m), 7.32-7.38 (3H, m), 7.49-7.51 (2H, m), 7.71 (1H, t), 8.85 (1H, s), 8.89 (1H, s); RT=4.02 min; m/z (ES⁺)=312.13 [M+H]⁺.

### Example 164

### 1-(4-Chlorophenyl)-3-(4-pyrrol-1-ylphenyl)urea

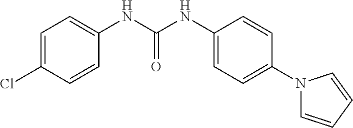

To a solution of 4-chlorophenylisocyanate (100 mg, 0.65 mmol) in CH₂Cl₂ (7 mL) was added 4-(1H-pyrrol-1-yl)aniline (103 mg, 0.65 mmol) at rt. The reaction mixture was stirred at rt for 16 h and the resulting solid collected by filtration. Trituration with Et₂O, followed by filtration gave the title compound: δ_{H} (DMSO): 6.23 (2H, t), 7.27 (2H, t), 7.32-7.34 (2H, m), 7.46-7.54 (6H, m), 8.77 (1H, s), 8.82 (1H, s); RT=4.01 min; m/z (ES⁺)=312.13 [M+H]⁺.

### Example 165

### 1-(4-Chlorophenyl)-3-[3-(2-methylpyrimidin-4-yl)phenyl]urea hydrochloride

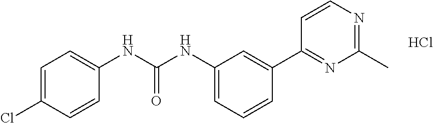

To a solution of 4-chlorophenylisocyanate (1.0 g, 6.5 mmol) in CH₂Cl₂ (20 mL) was added 3-(2-methylpyrimidin-4-yl)phenylamine (1.2 g, 6.5 mmol). The reaction mixture was stirred for 18 h and the resulting precipitate collected by filtration to yield 2.05 g (93%). The filtrate was dissolved in THF (10 mL) and 4M hydrogen chloride solution in dioxane (1.5 mL, 6.0 mmol) added. The resultant solid was filtered, dissolved in methanol and precipitated with Et₂O to yield, after filtration, the title compound: δ_{H} (CD₃OD): 2.96 (3H, s), 7.29 (2H, d), 7.47 (211, d), 7.54-7.58 (1H, m), 7.68 (1H, d), 8.03 (1H, d), 8.36 (1H, d), 8.63 (1H, s), 8.96 (1H, d); RT=3.45 min; m/z (ES⁺)=339.01 [M+H]⁺.

### Example 166

### 1-(4-Chlorophenyl)-3-[4-(3,4-dihydro-1H-isoquinolin-2-yl)-3-fluorophenyl]urea

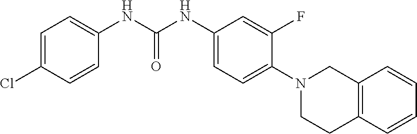

To a solution of 4-chlorophenylisocyanate (230 mg, 1.5 mmol) in CH₂Cl₂ (15 mL) was added 4-(3,4-dihydro-1H-isoquinolin-2-yl)-3-fluorophenylamine (363 mg, 1.5 mmol). The reaction mixture was stirred for 18 h and the resulting precipitate collected by filtration. Recrystallisation from MeOH gave the title compound: δ_{H} (DMSO): 2.91-2.94 (2H, m), 3.30-3.33 (2H, m), 4.18 (2H, s), 7.06-7.08 (2H, m), 7.18 (4H, s), 7.33-7.35 (2H, m), 7.48-7.50 (3H, m), 8.74 (1H, s), 8.81 (1H, s); RT=4.20 min; m/z (ES⁺)=396.11 [M+H]⁺.

### Example 167

### 1-(3,4-Dichlorophenyl)-3-(3-fluoro-4-piperidin-1-ylphenyl)urea

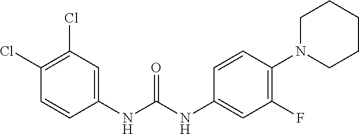

To a solution of 3,4-dichlorophenylisocyanate (282 mg, 1.50 mmol) in CH₂Cl₂ (7 mL) was added 3-fluoro-4-piperidin-1-yl-phenylamine (320 mg, 1.65 mmol). The reaction mixture was stirred for 18 h and the resulting precipitate collected by filtration to give the title compound: δ_{H} (DMSO): 1.51-1.53 (2H, m), 1.61-1.67 (4H, m), 2.87-2.90 (4H, m), 6.94-6.98 (1H, m), 7.04-7.06 (1H, m), 7.30-7.33 (1H, m), 7.35-7.40 (1H, m), 7.50-7.52 (1H, d), 7.86 (1H, m), 8.76 (1H, s), 8.95 (1H, s); RT=3.56 min; m/z (ES⁺)=381.98 [M+H]⁺.

### Example 168

### 1-(4-Chlorophenyl)-3-[3-(6-pyrrolidin-1-ylpyridin-2-yl)phenyl]urea

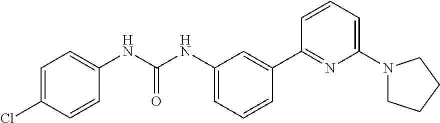

A suspension of 2-(3-nitrophenyl)-6-pyrrolidin-1-ylpyridine (2.83 g, 10.52 mmol) and 10% palladium on carbon (0.50 g) in EtOH (50 mL) and CH₂Cl₂ (30 mL) was stirred under an atmosphere of hydrogen for 18 h. The mixture was filtered through celite and the solvent removed under vacuum to give 3-(6-pyrrolidin-1-ylpyridin-2-yl)aniline, which was used without further purification. To a solution of 3-(6-pyrrolidin-1-ylpyridin-2-yl)aniline (1.01 g, 4.226 mmol) in CH₂Cl₂ (5 mL) was added 4-chlorophenyl isocyanate (0.59 g, 3.84 mmol). The reaction mixture was stirred for 18 h and the resulting precipitate collected by filtration. The solid was purified by recrystallisation (MeOH/CH₂Cl₂) to give the title compound: δ_{H} (CDCl₃): 1.97 (4H, m), 3.48 (4H, m), 6.42 (1H, d), 7.05 (1H, d), 7.33 (2H, d), 7.35 (1H, m), 7.50 (2H, d), 7.56 (2H, m), 7.63 (1H, d), 8.07 (1H, s), 8.78 (1H, s), 8.82 (1H, s); RT=3.19 min; m/z (ES⁺)=393.13 [M+H]⁺.

### Example 169

### 1-(4-Azepan-1-yl-3-fluorphenyl)-3-(4-chlorophenyl)urea

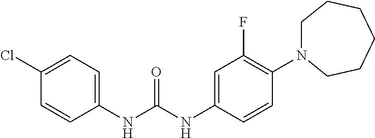

A mixture of 1-(2-fluoro-4-nitrophenyl)azepane (0.30 g, 1.26 mmol) and iron powder (0.22 g, 3.99 mmol) in saturated NH₄Cl (aq) (1.5 mL), THF (2 mL) and EtOH (4 mL) was heated at 80° C. for 20 h. The reaction was partitioned between CH₂Cl₂ and water, the organic phase was dried (MgSO₄) and the solvent removed to give 4-azepan-1-yl-3-fluorophenylamine. To a solution of 4-azepan-1-yl-3-fluorophenylamine (0.16 g, 0.779 mmol) in CH₂Cl₂ (2 mL) was added 4-chlorophenyl isocyanate (0.12 g, 0.779 mmol) and the reaction stirred for 18 h. The resulting precipitate was collected by filtration to give the title compound: δ_{H} (CDCl₃): 1.56 (4H, m), 1.74 (4H, m), 3.24 (4H, m), 6.86 (1H, m), 6.96 (1H, m), 7.30 (2H, d), 7.35 (1H, m), 7.46 (2H, d), 8.55 (1H, s), 8.73 (1H, s); RT=3.82 min; m/z (ES⁺)=362.06 [M+H]⁺.

### Example 170

### 1-(4-Chlorophenyl)-3-(3-fluoro-4-pyrrolidin-1-ylphenyl)urea

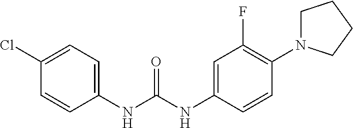

A mixture of 1-(2-fluoro-4-nitrophenyl)pyrrolidine (0.26 g, 1.26 mmol) and iron powder (0.22 g, 3.99 mmol) in saturated NH₄Cl (aq) (1.5 mL), THF (2 mL) and EtOH (4 mL) was heated at 80° C. for 20 h. The reaction was partitioned between CH₂Cl₂ and water, the organic phase was dried (MgSO₄) and the solvent removed to give 3-fluoro-4-pyrrolidin-1-yl-phenylamine. To a solution of 3-fluoro-4-pyrrolidin-1-yl-phenylamine (0.13 g, 0.71 mmol) in CH₂Cl₂ (2 mL) was added 4-chlorophenylisocyanate (0.11 g, 0.71 mmol) and the reaction stirred for 18 h. The resulting precipitate was collected by filtration to give the title compound: (CDCl₃): 1.88 (4H, m), 3.23 (4H, m), 6.69 (1H, m), 6.97 (1H, m), 7.30 (2H, d), 7.35 (1H, m), 7.46 (2H, d), 8.51 (1H, s), 8.71 (1H, s); RT=3.44 min; m/z (ES⁺)=334.04 [M+H]⁺.
The following compounds were also prepared by methods analogous to those described above:

### Example

171 1-(3-Fluoro-4-piperidin-1-ylphenyl)-3-[4-(2-morpholin-4-ylethoxy)phenyl]urea
172 1-(3-Fluoro-4-piperidin-1-ylphenyl)-3-[4-(2-methoxyethoxy)phenyl]urea
173 1-(4-Chlorophenyl)-3-[3-(2-isopropylpyrimidin-4-yl)phenyl]urea
174 1-(4-Chlorophenyl)-3-(3-fluoro-4-[1,4]oxazepan-4-ylphenyl)urea
175 1-(3-Fluoro-4-piperidin-1-ylphenyl)-3-[4-(morpholine-4-carbonyl)phenyl]urea
176 1-(3-Fluoro-4-piperidin-1-ylphenyl)-3-(4-pyrrol-1-ylphenyl)urea
177 4-[3-(3-Fluoro-4-piperidin-1-ylphenyl)ureido]benzoic acid ethyl ester
178 1-(3-Fluoro-4-piperidin-1-ylphenyl)-3-[3-(2-methoxyethoxy)phenyl]urea
179 1-(3-Fluoro-4-piperidin-1-ylphenyl)-3-[3-(2-morpholin-4-ylethoxy)phenyl]urea
180 1-(4-Chlorophenyl)-3-(4-pyridin-3-ylphenyl)urea
181 1-(4-Chlorophenyl)-3-[3-(6-methylpyrimidin-4-yl)phenyl]urea
182 1-(4-Chlorophenyl)-3-[3-fluoro-4-(3-hydroxypiperidin-1-yl)phenyl]urea
183 1-(4-Chlorophenyl)-3-(4-pyridin-2-yl-phenyl)urea
184 1-(4-Chlorophenyl)-3-(4-pyridin-4-ylphenyl)urea
185 1-(4-Chlorophenyl)-3-[3-(2-piperidin-1-ylpyrimidin-4-yl)phenyl]urea
186 1-(3-Fluoro-4-piperidin-1-ylphenyl)-3-[4-(2-morpholin-4-ylethyl)phenyl]urea
187 1-(3-Fluoro-4-piperidin-1-ylphenyl)-3-(4-morpholin-4-ylmethylphenyl)urea
188 1-(2,3-Dihydrobenzofuran-6-yl)-3-(3-fluoro-4-piperidin-1-ylphenyl)urea
189 1-(3,5-Dimethoxyphenyl)-3-(3-fluoro-4-piperidin-1-ylphenyl)urea
190 1-(4-Chlorophenyl)-3-(3-pyrazol-1-ylphenyl)urea
191 1-(4-Chlorophenyl)-3-[3-(3,4,5,6-tetrahydro-2H-[1,2]bipyridinyl-6′-yl)phenyl]urea
192 1-(4-Morpholin-4-ylmethylphenyl)-3-(4-pyrrol-1-ylphenyl)urea
193 1-(4-Morpholin-4-ylmethylphenyl)-3-(3-pyrrol-1-ylphenyl)urea
194 1-(4-Chlorophenyl)-3-[4-(4,4-difluoropiperidin-1-yl)-3-fluorophenyl]urea
195 1-(4-Butyrylphenyl)-3-[4-(morpholine-4-carbonyl)phenyl]urea
196 1-(1-Methyl-1H-indazol-5-yl)-3-(4-morpholin-4-ylmethylphenyl)urea
197 1-(4-Morpholin-4-ylmethylphenyl)-3-(4-pyrazol-1-ylphenyl)urea
198 1-(2,3-Dihydrobenzo[1,4]dioxin-6-yl)-3-(3-fluoro-4-piperidin-1-ylphenyl)urea
199 1-(3,5-Dichlorophenyl)-3-(3-fluoro-4-piperidin-1-ylphenyl)urea
200 1-(3-Chloro-4-fluorophenyl)-3-(3-fluoro-4-piperidin-1-ylphenyl)urea
201 1-(4-Ethylphenyl)-3-(3-fluoro-4-piperidin-1-ylphenyl)urea
202 1-[2-(4-Chlorophenyl)ethyl]-3-[3-(2-methyl-2H-pyrazol-3-yl)phenyl]urea
203 1-(4-Chlorophenyl)-3-[3-fluoro-4-(4-hydroxypiperidin-1-yl)phenyl]urea
204 1-(4-Chlorophenyl)-3-[3-fluoro-4-(3-methylpiperidin-1-yl)phenyl]urea
205 1-Benzo[1,3]dioxol-5-yl-3-(3-fluoro-4-piperidin-1-ylphenyl)urea
206 1-(4-Chlorophenyl)-3-[3-fluoro-4-(2-methylpiperidin-1-yl)phenyl]urea
207 1-(3-Fluoro-4-piperidin-1-ylphenyl)-3-(4-methoxyphenyl)urea
208 1-(4-Chloro-2-hydroxyphenyl)-3-(3-fluoro-4-piperidin-1-ylphenyl)urea
209 1-(3-Fluoro-4-piperidin-1-ylphenyl)-3-[3-(2-methylpyrimidin-4-yl)phenyl]urea
210 1-(4-Chlorophenyl)-3-[3-fluoro-4-(4-trifluoromethylpiperidin-1-yl)phenyl]urea
211 1-(4-Chlorophenyl)-3-[3-fluoro-4-(4-methylpiperidin-1-yl)phenyl]urea
212 1-(3-Fluoro-4-piperidin-1-ylphenyl)-3-(4-phenoxyphenyl)urea
213 1-(3-Fluoro-4-piperidin-1-ylphenyl)-3-(3-phenoxyphenyl)urea
214 1-(4-Fluorophenyl)-3-(3-fluoro-4-piperidin-1-ylphenyl)urea
215 1-(3-Fluoro-4-piperidin-1-ylphenyl)-3-(3-methoxyphenyl)urea
216 1-(4-Cyanophenyl)-3-(3-fluoro-4-piperidin-1-ylphenyl)urea
217 1-(4-Chlorophenyl)-3-(4-morpholin-4-ylmethylphenyl)urea
218 1-(4-Chloro-3-trifluoromethylphenyl)-3-(3-fluoro-4-piperidin-1-ylphenyl)urea
219 1-(3-Fluoro-4-piperidin-1-ylphenyl)-3-(4-trifluoromethylphenyl)urea
220 1-(3-Chlorophenyl)-3-(3-fluoro-4-piperidin-1-ylphenyl)urea
221 1-(4-Chlorophenyl)-3-(3,4,5,6-tetrahydro-2H-[1,2′]bipyridinyl-5′-yl)urea
222 1-(4-Chlorophenyl)-3-[4-(morpholine-4-carbonyl)phenyl]urea
223 1-(4-Chlorophenyl)-3-(3-dimethylaminophenyl)urea
224 1-(4-Chlorophenyl)-3-(3-fluoro-4-morpholin-4-ylphenyl)urea
225 1-[2-(4-Chlorophenyl)ethyl]-3-(3-pyrrol-1-ylphenyl)urea
226 1-(3,5-Dichlorophenyl)-3-(3,4,5,6-tetrahydro-2H-[1,2′]bipyridinyl-5′-yl)urea
227 1-(3-Chlorophenyl)-3-(3,4,5,6-tetrahydro-2H-[1,2′]bipyridinyl-5′-yl)urea
228 1-(3,5-Bis-trifluoromethylphenyl)-3-(3,4,5,6-tetrahydro-2H-[1,2′]bipyridinyl-5′-yl)urea
229 1-(4-Acetylphenyl)-3-[4-(morpholine-4-carbonyl)phenyl]urea
230 1-(4-Acetylphenyl)-3-[3-(6-methoxypyridin-2-yl)phenyl]urea
231 1-(4-Acetylphenyl)-3-(4-morpholin-4-ylmethylphenyl)urea
232 1-(4-Chlorophenyl)-3-(3-fluoro-4-piperidin-1-ylphenyl)urea
233 1-(3-Chloro-4-morpholin-4-ylphenyl)-3-(4-chlorophenyl)urea
234 1-(4-Chlorophenyl)-3-(4-piperidin-1-ylphenyl)urea
235 1-(4-Acetylphenyl)-3-(3,4,5,6-tetrahydro-2H-[1,2′]bipyridinyl-5′-yl)urea
236 1-(4-Butyrylphenyl)-3-(4-piperidin-1-ylphenyl)urea
237 1-[2-(4-Chlorophenyl)ethyl]-3-(4-morpholin-4-ylmethylphenyl)urea
238 1-(4-Chlorophenyl)-3-(1-methyl-1H-indazol-5-yl)urea
239 1-(3-Acetylphenyl)-3-[2-(4-chlorophenyl)ethyl]urea
240 1-(4-Chlorophenyl)-3-[3-(2-pyrrolidin-1-ylpyrimidin-4-yl)phenyl]urea
241 1-(4-Chlorophenyl)-3-(4-pyrazol-1-ylphenyl)urea
242 1-[2-(4-Chlorophenyl)ethyl]-3-[4-(morpholine-4-carbonyl)phenyl]urea

The biological activity of the compounds of the invention may be tested in the following assay systems:

### The Yeast GPCR Antagonism Assay:

Yeast cells harboring the CB1 receptor gene and a FUS1p-LacZ transcriptional reporter on plasmids are grown at 30° C. in selective minimal media buffered by Pipes buffer to pH 6.8. Following overnight incubation, yeast cells are harvested by centrifugation at 1000 g for 10 min, then resuspended in fresh buffered media to a cell density of A₆₀₀=0.3. To set up the assay, 80 μL of cells are inoculated into a 96-well flat bottom black plate containing 10 μL of a range of dilution of test compounds in 10% DMSO, 0.5% BSA solution. The range of compound concentrations used for the dose response curve is usually 1 nM-10 μM. After a 15 min incubation period at 30° C., 10 μL of CB1 agonist (methanandamide or CP 55,940) is added to a final concentration of 2 μM or 0.1 μM respectively. The assay plates are then incubated at 30° C. for a further 4 h. At the end of this period, β-galactosidase enzyme activity within the cells is assayed fluorometrically by the addition 83 μM of the substrate 4-methylumbelliferyl-β-D-galactopyranoside (MUG) in a 20 μL volume of buffer containing 25 mM Pipes pH 7.2 and 0.41% Triton X-100. The reaction is allowed to proceed for 45 min at 30° C. before being stopped by the addition of 20 μL of 1M Na₂CO₃. MUG's hydrolysis product, 13-methylumbelliferone (7-hydroxy-4-methylcoumarin), is measured via its fluorescence emission at 460 nM following excitation at 360 nM. The IC₅₀ for each compound is then calculated as the concentration of compound needed to reduce the fluorescence increase, due to the addition of agonist, by 50%.

Competitive GTPγS binding assay:

Membrane preparations of the human CB1 receptor expressed in HEK293 EBNA cells were purchased from PerkinElmer life sciences. Binding experiments were carried out in 96-round bottom plates in a total volume of 200 μL of buffer A (20 mM Hepes, 3 mM MgCl₂, 100 mM NaCl, 1 mM EDTA, 0.1% BSA, pH 7.4) containing, in addition, 20 μg of membrane, 0.1 nM [³⁵S] GTPγS (sp.act. 1250 Ci/mmole), 50 nM agonist CP-55940 (Tocris), 10 μM GDP and the required range of antagonist concentrations made up in DMSO to give a final DMSO concentration of 1%.

Following incubation for 1 h at 30° C., the reactions were transferred to a 96-well GF/B MAFB filter plate (Millipore) pre-soaked in 20 mM Hepes, 3 mM MgCl₂, 100 mM NaCl and 1 mM EDTA, pH 7.4. The plate was then filtered and washed with 4×250 μL volumes of ice cold buffer A using a Multiscreen vacuum manifold (Millipore). After drying at 50° C. for 2 h, 30 μL of scintillant (Ultima Gold™, Packard) was added to each well and the plate counted for radioactivity in a Packard MicroBeta counter. Non-specific binding was determined by the addition of 30 μM GTPγS in place of antagonist. Basal [³⁵S] GTPγS binding determined in absence of agonist and antagonist and Maximal [³⁵S] GTPγS binding determined in presence of agonist but in absence of antagonist. IC₅₀'s were calculated from plots of % reduction in agonist stimulated [³⁵S] GTPγS binding versus log₁₀ antagonist concentrations using the Xlfit3 program (idbs). IC₅₀ being the concentration of antagonist required to reduce agonist stimulated [³⁵S] GTPγS binding by 50%.

The Examples of the present invention generally demonstrated efficacy in the above assays with IC₅₀ results better than 10 μM. It is advantageous that the IC₅₀ be better than 5 μM, even more advantageous if better than 1 μM, and still more advantageous if better than 300 nM.

1. A method of treating a condition associated with the CB-1 receptor by administering to a subject in need of such treatment a compound of formula (I): 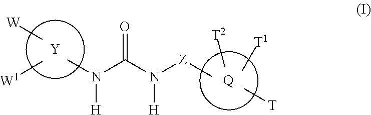
or a pharmaceutically acceptable salt thereof, wherein:
Y is phenyl, a 5- or 6-membered heteroaryl group, or a 9-membered bicyclic heteroaryl group attached to the urea through the 5-membered ring;
W is COOR¹, COR¹, C₁₋₃alkyl, C₁₋₃-fluoroalkyl, C₁₋₆alkoxy, phenoxy, C₁₋₃-fluoroalkoxy, C₁₋₃alkoxyC₁₋₃alkoxy, C₁₋₆alkylthio, C₃₋₆cycloalkyl, chloro, fluoro, nitrile, —(CH₂)ₘ—NR²R³, —O(CH₂)ₘ—NR²R³, or 5- or 6-membered heteroaryl optionally substituted by 1 or 2 groups independently selected from C₁₋₃alkyl, C₁₋₃-fluoroalkyl, C₁₋₃alkoxy, C₁₋₃-fluoroalkoxy, C₁₋₃alkoxyC₁₋₃alkyl, chloro, fluoro and —(CH₂)ₘ—NR²R³; or when Y is a 9-membered bicyclic heteroaryl group attached to the urea through the 5-membered ring, or when Z is C₁₋₃alkylene or C₂₋₃alkenylene, then W may be hydrogen;
W¹ is hydrogen, halogen, C₁₋₃ alkyl, hydroxy or C₁₋₃alkoxy;
or W and W¹, when attached to adjacent carbon atoms on Y, together form a group —O—(CH₂)ₚ—O—, wherein p is 1, 2 or 3;
or the group formed from —Y, -(W) and -(W¹) is: 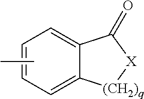
wherein X is O or CH₂ and q is 1 or 2;
Z is C₁₋₃alkylene, C₂₋₃alkenylene or a bond;
Q is phenyl, or a 5- to 10-membered mono- or bicyclic heteroaryl group;
T is hydrogen, halogen, nitro, nitrile, COOR¹, COR¹, —(CH₂)ₘ—NR²R³, CONHCH₂COOH, C₁₋₆alkyl optionally substituted by COOR⁴ or OR⁴, C₁₋₃ fluoroalkyl, C₁₋₆alkoxy, C₁₋₃-fluoroalkoxy, C₁₋₆alkylthio, SOR⁵, SO₂R⁵; or a C₃₋₃cycloalkyl group, 5- to 7-membered heterocyclyl group or 5- to 10-membered heteroaryl group any one of which is optionally substituted by 1 or 2 groups independently selected from C₁₋₃alkyl, C₁₋₃-fluoroalkyl, C₁₋₃alkoxy, C₁₋₃-fluoroalkoxy, C₁₋₃alkoxyC₁₋₃alkyl, chloro, fluoro, hydroxy and —(CH₂)ₘ—NR²R³;
T¹ and T² are independently selected from hydrogen, halogen, hydroxy, C₁₋₃alkyl and C₁₋₃alkoxy;
or T and T¹, when attached to adjacent carbon atoms on Q, together form a group —O—(CH₂)ₚ—O—, wherein p is 1, 2 or 3;
m is 0, 1, 2 or 3;
n is 2 or 3;
R¹ is C₁₋₆alkyl, C₃₋₆cycloalkyl, phenyl or a 5- or 6-membered heteroaryl or heterocyclyl group;
R² and R³ are independently selected from hydrogen, C₁₋₆alkyl and C₃₋₆cycloalkyl, or R² and R³ together with the nitrogen to which they are attached form a 5- to 7-membered heterocyclic ring optionally containing an additional heteroatom selected from O, S and NR⁴, and optionally substituted by 1 or 2 groups independently selected from C₁₋₃alkyl, fluoro and hydroxyl;
R⁴ is hydrogen or C₁₋₃alkyl; and
R⁵ is C₁₋₆alkyl or C₃₋₆cycloalkyl.

2. The method according to claim 1 wherein when Y is phenyl.

3. The method according to claim 1 wherein when Y is a 5- or 6-membered heteroaryl group it is thienyl, thiazolyl or thiadiazolyl.

4. The method according to claim 1 wherein when Y is a 9-membered bicyclic heteroaryl group it is benzothienyl or benzothiazolyl.

5. The method according to claim 2 wherein W is COOR¹, COR¹, C₁₋₆alkoxy, C₁₋₆alkylthio, fluoro, chloro, C₁₋₃alkoxyC₁₋₃alkoxy, —(CH₂)ₘ—NR²R³, —O(CH₂)ₙ—NR²R³, or 5- or 6-membered heteroaryl optionally substituted by C₁₋₃alkyl.

6. The method according to claim 1 wherein W¹ is hydrogen.

7. The method according to claim 1 wherein Z is C₂alkylene, C₂alkenylene or a bond.

8. The method according to claim 7 wherein Z is a bond.

9. The method according to claim 1 wherein Q is phenyl.

10. The method according to claim 1 wherein T is halogen, COOR¹, COR¹, C₁₋₆alkyl, —(CH₂)ₘ—NR²R³ optionally substituted by 1 or 2 groups independently selected from C₁₋₃alkyl, fluoro and hydroxy, or a 5- to 10-membered heteroaryl group optionally substituted by C₁₋₃alkyl.

11. The method according to claim 10 wherein when T is —(CH₂)ₘ—NR²R³, m is 0 and R² and R³ together with the nitrogen to which they are attached form a 5- to 7-membered heterocyclic ring.

12. The method according to claim 1 wherein T¹ and T² are hydrogen, halogen or hydroxy.

13. The method according to claim 12 wherein T² is hydrogen.

14. The method according to claim 1 wherein the substituents on the groups Y and Q are in the meta and/or para positions relative to the urea.

15. The method according to claim 1 wherein the compound of formula (I) is the compound of any one of Examples 1 to 242, or a pharmaceutically acceptable salt thereof.

16. The method according to claim 1 for the treatment of obesity; psychiatric disorders such as psychotic disorders, schizophrenia, bipolar disorders, depression, cognitive disorders, memory disorders, obsessive compulsive disorders, anorexia, bulimia, attention disorders, epilepsy and related conditions affective and cognitive disorders brought about by disturbances in any of the central monoaminergic systems; a neurological disorder such as Raynaud's syndrome, movement impairment, Parkinson's disease, Huntington's chorea or Alzheimer's disease; immune, cardiovascular, reproductive and endocrine disorders, endotoxin-induced or cirrhotic hypotension, septic shock, diseases related to the respiratory and gastrointestinal systems such as decreased intestinal motility such as Paralytic ileus caused by peritonitis, surgery, or other noxious situations, extended abuse, addiction and relapse indications such as tobacco smoking, heroin addiction, relapse to cocaine-seeking, or alcoholism.

17. The method according to claim 16 wherein the condition associated with the CB-1 receptor is obesity.

18. A compound of formula (Ia): 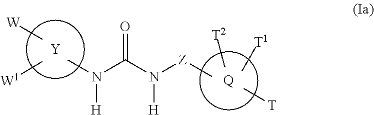
or a pharmaceutically acceptable salt thereof, wherein:
Y is phenyl, a 5- or 6-membered heteroaryl group, or a 9-membered bicyclic heteroaryl group attached to the urea through the 5-membered ring;
W is COOR¹, COR¹, C₁₋₆alkoxy, C₁₋₃ fluoroalkoxy, C₁₋₃alkoxyC₁₋₃alkoxy, —(CH₂)ₘ—NR²R³, —O(CH₂)ₘ—NR²R³, C₁₋₆alkylthio, fluoro, chloro or 5- or 6-membered heteroaryl optionally substituted by C₁₋₃alkyl,
W¹ is hydrogen, halogen or C₁₋₃alkoxy;
Z is C₁₋₃alkylene, C₂₋₃alkenylene or a bond;
Q is phenyl, pyridyl or a 9-membered bicyclic heteroaryl group;
T is halogen, COOR¹, COR¹, C₁₋₆alkyl, —(CH₂)ₘ—NR²R³, or a 5- to 10-membered heteroaryl group optionally substituted by C₁₋₃alkyl; or when Z is C₁₋₃alkylene or C₂₋₃-alkenylene, then T may be hydrogen;
T¹ and T² are independently selected from hydrogen, halogen and hydroxy;
R¹ is C₁₋₆alkyl or phenyl or a 5- or 6-membered heteroaryl or heterocyclyl group;
R² and R³ together with the nitrogen to which they are attached form a 5- to 7-membered heterocyclic ring optionally containing an additional heteroatom selected from O, S and NR⁴, and optionally substituted by 1 or 2 groups independently selected from C₁₋₃alkyl, fluoro and hydroxy;
m is 0, 1, 2 or 3; and
n is 2 or 3;
provided that the compound is not:
1-Benzo[b]thiophen-2-yl-3-(2-methylphenyl)urea,
4-[3-(2-Chlorophenyl)ureido]benzoic acid ethyl ester,
4-[3-(4-Methylsulfanylphenyl)ureido]benzoic acid ethyl ester,
4-[3-(4-Chlorophenyl)ureido]benzoic acid ethyl ester,
1-(3-Chlorophenyl)-3-(4-ethoxyphenyl)urea,
4-[3-(3-Chlorophenyl)ureido]benzoic acid ethyl ester,
1,3-Bis(4-acetylphenyl)urea,
4-[3-(4-Fluorophenyl)ureido]benzoic acid ethyl ester,
1-(4-Fluorophenyl)-3-(4-methoxyphenyl)urea,
1-(4-Acetylphenyl)-3-(3-chlorophenyl)urea,
1-(4-Acetylphenyl)-3-(4-chlorophenyl)urea,
1-(4-Chlorophenyl)-3-(4-ethoxyphenyl)urea,
1-(4-Acetylphenyl)-3-(4-fluorophenyl)urea,
4-[3-(4-Fluorophenyl)ureido]benzoic acid methyl ester,
4-[3-(3-Fluorophenyl)ureido]benzoic acid ethyl ester,
4-[3-(2-Fluorophenyl)ureido]benzoic acid ethyl ester,
1-(3-Ethoxyphenyl)-3-(4-fluorophenyl)urea,
1-(4-Chlorophenyl)-3-(4-trifluoromethoxyphenyl)urea,
1-(3-Acetylphenyl)-3-[2-(4-chlorophenyl)ethyl]urea,
1-(4-Chlorophenyl)-3-[4-(morpholine-4-carbonyl)phenyl]urea, or
1,3-Bis(3,4-dichlorophenyl)urea.

19. A compound according to claim 18 wherein when Y is phenyl.

20. A compound according to claim 18 wherein when Y is a 5- or 6-membered heteroaryl group it is thienyl, thiazolyl or thiadiazolyl.

21. A compound according to claim 18 wherein when Y is a 9-membered bicyclic heteroaryl group it is benzothienyl or benzothiazolyl.

22. A compound according to claim 19 wherein W is COOR¹, COR¹, C₁₋₆alkoxy, C₁₋₆-alkylthio, fluoro, chloro, C₁₋₃alkoxyC₁₋₃alkoxy, —(CH₂)ₘ—NR²R³, —O(CH₂)ₙ—NR²R³, or 5- or 6-membered heteroaryl optionally substituted by C₁₋₃alkyl.

23. A compound according to claim 18 wherein W¹ is hydrogen.

24. A compound according to claim 18 wherein Z is C₂alkylene, C₂alkenylene or a bond.

25. A compound according to claim 24 wherein Z is a bond.

26. A compound according to claim 18 wherein Q is phenyl.

27. A compound according to claim 18 wherein T is halogen, COOR¹, COR¹, C₁₋₆alkyl, —(CH₂)ₘ—NR²R³ optionally substituted by 1 or 2 groups independently selected from C₁₋₃alkyl, fluoro and hydroxy, or a 5- to 10-membered heteroaryl group optionally substituted by C₁₋₃alkyl.

28. A compound according to claim 27 wherein when T is —(CH₂)ₘ—NR²R³, m is 0 and R² and R³ together with the nitrogen to which they are attached form a 5- to 7-membered heterocyclic ring.

29. A compound according to claim 18 wherein T¹ and T² are hydrogen, halogen or hydroxy.

30. A compound according to claim 29 wherein T² is hydrogen.

31. A compound according to claim 18 wherein the substituents on the groups Y and Q are in the meta and/or para positions relative to the urea.

32. A compound selected from:
2-[3-(4-Fluorophenyl)ureido]-4-methylthiazole-5-carboxylic acid ethyl ester
2-[3-(3-Fluorophenyl)ureido]-4-methylthiazole-5-carboxylic acid ethyl ester
2-[3-(4-Ethoxycarbonylphenyl)ureido]-4-methylthiazole-5-carboxylic acid ethyl ester
4-Methyl-2-[3-(4-methylsulfanylphenyl)ureido]thiazole-5-carboxylic acid ethyl ester
1-(4-Acetylphenyl)-3-benzo[b]thiophen-2-ylurea
1-Benzo[b]thiophen-2-yl-3-(4-methanesulfonylphenyl)urea
4-[3-(4-Fluoro-2-methylphenyl)ureido]benzoic acid ethyl ester
4-[3-(2,4,6-Trifluorophenyl)ureido]benzoic acid ethyl ester
4-[3-(2,4-Difluorophenyl)ureido]benzoic acid ethyl ester
4-[3-(3,4-Difluorophenyl)ureido]benzoic acid ethyl ester
4-[3-(2-Chloro-4-fluorophenyl)ureido]benzoic acid ethyl ester
4-[3-(4-Fluoro-3-methylphenyl)ureido]benzoic acid ethyl ester
4-[3-(3-Chloro-4-fluorophenyl)ureido]benzoic acid ethyl ester
4-[3-(4-Fluoro-3-methoxyphenyl)ureido]benzoic acid ethyl ester
1-(4-Ethoxyphenyl)-3-(4-fluorophenyl)urea
4-[3-(4-Fluorophenyl)ureido]-3-methylbenzoic acid methyl ester
4-[3-(4-Fluorophenyl)ureido]-3-hydroxybenzoic acid methyl ester
1-(2-Thiophen-2-ylethyl)-3-(4-methylphenyl)urea
1-(4-Methoxyphenyl)-3-(2-thiophen-2-ylethyl)urea
1-(2-Thiophen-2-ylethyl)-3-(4-trifluoromethoxyphenyl)urea
1-(4-Difluoromethoxyphenyl)-3-(2-thiophen-2-ylethyl)urea
1-(4-Ethylphenyl)-3-(2-thiophen-2-ylethyl)urea
1-(2-Thiophen-2-ylethyl)-3-(4-trifluoromethylphenyl)urea
1-(3-Chlorophenyl)-3-(2-thiophen-2-ylethyl)urea
1-(4-Butylphenyl)-3-(2-thiophen-2-ylethyl)urea
1-(4-Acetylphenyl)-3-(2-thiophen-2-ylethyl)urea
1-(3-Ethylphenyl)-3-(2-thiophen-2-ylethyl)urea
1-(4-Fluorophenyl)-3-(2-thiophen-2-ylethyl)urea
1-(4-Chlorophenyl)-3-(2-thiophen-2-ylethyl)urea
1-(4-Methylsulfanylphenyl)-3-(2-thiophen-2-ylethyl)urea
1-(4-Isopropylphenyl)-3-(2-thiophen-2-ylethyl)urea
4-(3-Benzothiazol-6-ylureido)benzoic acid ethyl ester
4-[3-(4-Imidazol-1-ylphenyl)ureido]benzoic acid ethyl ester
4-[3-(6-Fluorobenzothiazol-2-yl)ureido]benzoic acid ethyl ester
5-[3-(4-Ethoxycarbonylphenyl)ureido]furan-2-carboxylic acid methyl ester
4-[3-(1H-Indol-6-yl)ureido]benzoic acid ethyl ester
4-[3-(3-Methoxycarbonylphenyl)ureido]benzoic acid ethyl ester
2-[3-(4-Ethoxycarbonylphenyl)ureido]thiophene-3-carboxylic acid methyl ester
4-{3-[2-(1-Methyl-1H-pyrrol-2-yl)ethyl]ureido}benzoic acid ethyl ester
4-[3-(6-Methoxypyridin-3-yl)ureido]benzoic acid ethyl ester
6-[3-(4-Ethoxycarbonylphenyl)ureido]nicotinic acid methyl ester
4-[3-(6-Chloropyridin-3-yl)ureido]benzoic acid ethyl ester
4-[3-(4-Carboxymethylphenyl)ureido]benzoic acid ethyl ester
4-[3-(1H-Indol-5-yl)ureido]benzoic acid ethyl ester
1-(4-Fluorophenyl)-3-(4-morpholin-4-ylphenyl)urea
1-Benzothiazol-6-yl-3-(4-fluorophenyl)urea
1-(4-Fluorophenyl)-3-(4-imidazol-1-ylphenyl)urea
6-[3-(4-Fluorophenyl)ureido]nicotinic acid methyl ester
1-(6-Chlorobenzothiazol-2-yl)-3-(4-fluorophenyl)urea
1-(6-Fluorobenzothiazol-2-yl)-3-(4-fluorophenyl)urea
1-(4,6-Difluorobenzothiazol-2-yl)-3-(4-fluorophenyl)urea
1-(4-Fluorophenyl)-3-(6-methoxybenzothiazol-2-yl)urea
1-(4-Fluorophenyl)-3-[3-(2-methylpyrimidin-4-yl)phenyl]urea
3-[3-(4-Fluorophenyl)ureido]benzoic acid methyl ester
1-(4-Fluorophenyl)-3-(2-fluorophenyl)urea
3-[3-(4-Fluorophenyl)ureido]benzoic acid ethyl ester
1-(4-Fluoro-3-methylphenyl)-3-(4-fluorophenyl)urea
4-{3-[3-(2-Methylpyrimidin-4-yl)phenyl]ureido}benzoic acid ethyl ester
4-[3-(1-Oxoindan-5-yl)ureido]benzoic acid ethyl ester
4-[3-(6-Morpholin-4-ylpyridin-3-yl)ureido]benzoic acid ethyl ester
2-[3-(4-Ethoxycarbonylphenyl)ureido]thiazole-5-carboxylic acid methyl ester
4-[3-(3-Ethoxycarbonylphenyl)ureido]benzoic acid ethyl ester
4-[3-(4-Ethoxycarbonylphenyl)ureido]benzoic acid propyl ester
4-[3-(4-Ethoxycarbonylphenyl)ureido]benzoic acid pentyl ester
4-[3-(4-Ethoxycarbonylphenyl)ureido]benzoic acid isobutyl ester
4-[3-(4-Ethoxycarbonylphenyl)ureido]benzoic acid phenyl ester
4-{3-[4-(1,1,2,2-Tetrafluoroethoxy)phenyl]ureido}benzoic acid ethyl ester
4-[3-(3-Oxazol-5-ylphenyl)ureido]benzoic acid ethyl ester
4-[3-(4-Ethoxycarbonylmethylphenyl)ureido]benzoic acid ethyl ester
4-[3-(4-[1,2,3]Thiadiazol-4-ylphenyl)ureido]benzoic acid ethyl ester
4-[3-(4-Propionylphenyl)ureido]benzoic acid ethyl ester
4-[3-(4-Acetylphenyl)ureido]benzoic acid ethyl ester
4-[3-(4-Benzoylphenyl)ureido]benzoic acid ethyl ester
4-{3-[4-(4,5-Dihydrooxazol-2-yl)phenyl]ureido}benzoic acid ethyl ester
4-{3-[4-(2-Methylpyrimidin-4-yl)phenyl]ureido}benzoic acid ethyl ester
1-(4-Fluorophenyl)-3-(4-pyrrol-1-ylphenyl)urea
1-(4-Fluorophenyl)-3-(2-methylbenzothiazol-5-yl)urea
1-(4-Fluorophenyl)-3-(3-oxazol-5-ylphenyl)urea
1-(4-Fluorophenyl)-3-(4-propionylphenyl)urea
1-(4-Fluorophenyl)-3-[4-(2-methylpyrimidin-4-yl)phenyl]urea
4-[3-(4-Ethoxycarbonylphenyl)ureido]benzoic acid butyl ester
2-[3-(4-Ethoxycarbonylphenyl)ureido]-4-methylpyrimidine-5-carboxylic acid ethyl ester
4-[3-(4-Oxazol-5-yl phenyl)ureido]benzoic acid ethyl ester
2-Chloro-4-[3-(4-ethoxycarbonylphenyl)ureido]benzoic acid ethyl ester
4-[3-(4-Ethoxycarbonylphenyl)ureido]-2-methoxybenzoic acid ethyl ester
4-[3-(4-Ethoxycarbonylphenyl)ureido]-3-methoxybenzoic acid ethyl ester
6-[3-(4-Ethoxycarbonylphenyl)ureido]nicotinic acid ethyl ester
4-[3-(4-Fluorophenyl)ureido]-3-hydroxy benzoic acid ethyl ester
4-[3-(3-Acetylphenyl)ureido]benzoic acid ethyl ester
4-[3-(4-Butyrylphenyl)ureido]benzoic acid ethyl ester
4-{3-[4-(1H-Pyrazol-3-yl)phenyl]ureido}benzoic acid ethyl ester
4-[3-(4-Fluorophenyl)ureido]benzoic acid propyl ester
4-[3-(4-Fluorophenyl)ureido]benzoic acid pentyl ester
4-[3-(4-Fluorophenyl)ureido]benzoic acid isobutyl ester
4-[3-(4-Fluorophenyl)ureido]benzoic acid phenyl ester
{4-[3-(4-Fluorophenyl)ureido]phenyl}acetic acid ethyl ester
1-(4-Benzoylphenyl)-3-(4-fluorophenyl)urea
1-(4-Butyrylphenyl)-3-(4-fluorophenyl)urea
4-[3-(4-Fluorophenyl)ureido]benzoic acid butyl ester
2-Chloro-4-[3-(4-fluorophenyl)ureido]benzoic acid ethyl ester
1-[2-(3-Fluorophenyl)ethyl]-3-(4-isopropylphenyl)urea
1-[2-(2-Fluorophenyl)ethyl]-3-(4-isopropylphenyl)urea
1-[2-(3-Fluorophenyl)ethyl]-3-(4-trifluoromethylphenyl)urea
1-(4-Isopropylphenyl)-3-thiazol-2-ylurea
1-(4-Acetylphenyl)-3-(4-bromophenyl)urea
1-[2-(4-Chlorophenyl)ethyl]-3-[3-(2-methylpyrimidin-4-yl)phenyl]urea
1-(4-Chlorophenyl)-3-(3-pyrrol-1-ylphenyl)urea
1-(4-Chlorophenyl)-3-(4-pyrrol-1-ylphenyl)urea
1-(4-Chlorophenyl)-3-[3-(2-methylpyrimidin-4-yl)phenyl]urea
1-(4-Chlorophenyl)-3-[4-(3,4-dihydro-1H-isoquinolin-2-yl)-3-fluorophenyl]urea
1-(3,4-Dichlorophenyl)-3-(3-fluoro-4-piperidin-1-ylphenyl)urea
1-(4-Chlorophenyl)-3-[3-(6-pyrrolidin-1-ylpyridin-2-yl)-phenyl]urea
1-(4-Azepan-1-yl-3-fluorophenyl)-3-(4-chlorophenyl)urea
1-(4-Chlorophenyl)-3-(3-fluoro-4-pyrrolidin-1-ylphenyl)urea
1-(3-Fluoro-4-piperidin-1-ylphenyl)-3-[4-(2-morpholin-4-ylethoxy)phenyl]urea
1-(3-Fluoro-4-piperidin-1-ylphenyl)-3-[4-(2-methoxyethoxy)phenyl]urea
1-(4-Chlorophenyl)-3-[3-(2-isopropylpyrimidin-4-yl)phenyl]urea
1-(4-Chlorophenyl)-3-(3-fluoro-4-[1,4]oxazepan-4-ylphenyl)urea
1-(3-Fluoro-4-piperidin-1-ylphenyl)-3-[4-(morpholine-4-carbonyl)phenyl]urea
1-(3-Fluoro-4-piperidin-1-ylphenyl)-3-(4-pyrrol-1-ylphenyl)urea
4-[3-(3-Fluoro-4-piperidin-1-ylphenyl)ureido]benzoic acid ethyl ester
1-(3-Fluoro-4-piperidin-1-ylphenyl)-3-[3-(2-methoxyethoxy)phenyl]urea
1-(3-Fluoro-4-piperidin-1-ylphenyl)-3-[3-(2-morpholin-4-ylethoxy)phenyl]urea
1-(4-Chlorophenyl)-3-(4-pyridin-3-ylphenyl)urea
1-(4-Chlorophenyl)-3-[3-(6-methylpyrimidin-4-yl)phenyl]urea
1-(4-Chlorophenyl)-3-[3-fluoro-4-(3-hydroxypiperidin-1-yl)phenyl]urea
1-(4-Chlorophenyl)-3-(4-pyridin-2-yl-phenyl)urea
1-(4-Chlorophenyl)-3-(4-pyridin-4-ylphenyl)urea
1-(4-Chlorophenyl)-3-[3-(2-piperidin-1-ylpyrimidin-4-yl)phenyl]urea
1-(3-Fluoro-4-piperidin-1-ylphenyl)-3-[4-(2-morpholin-4-ylethyl)phenyl]urea
1-(3-Fluoro-4-piperidin-1-ylphenyl)-3-(4-morpholin-4-ylmethylphenyl)urea
1-(2,3-Dihydrobenzofuran-6-yl)-3-(3-fluoro-4-piperidin-1-ylphenyl)urea
1-(3,5-Dimethoxyphenyl)-3-(3-fluoro-4-piperidin-1-ylphenyl)urea
1-(4-Chlorophenyl)-3-(3-pyrazol-1-ylphenyl)urea
1-(4-Chlorophenyl)-3-[3-(3,4,5,6-tetrahydro-2H-[1,2′]bipyridinyl-6′-yl)phenyl]urea
1-(4-Morpholin-4-ylmethylphenyl)-3-(4-pyrrol-1-ylphenyl)urea
1-(4-Morpholin-4-ylmethylphenyl)-3-(3-pyrrol-1-ylphenyl)urea
1-(4-Chlorophenyl)-3-[4-(4,4-difluoropiperidin-1-yl)-3-fluorophenyl]urea
1-(4-Butyrylphenyl)-3-[4-(morpholine-4-carbonyl)phenyl]urea
1-(1-Methyl-1H-indazol-5-yl)-3-(4-morpholin-4-ylmethylphenyl)urea
1-(4-Morpholin-4-ylmethylphenyl)-3-(4-pyrazol-1-ylphenyl)urea
1-(2,3-Dihydrobenzo[1,4]dioxin-6-yl)-3-(3-fluoro-4-piperidin-1-ylphenyl)urea
1-(3,5-Dichlorophenyl)-3-(3-fluoro-4-piperidin-1-ylphenyl)urea
1-(3-Chloro-4-fluorophenyl)-3-(3-fluoro-4-piperidin-1-ylphenyl)urea
1-(4-Ethylphenyl)-3-(3-fluoro-4-piperidin-1-ylphenyl)urea
1-[2-(4-Chlorophenyl)ethyl]-3-[3-(2-methyl-2H-pyrazol-3-yl)phenyl]urea
1-(4-Chlorophenyl)-3-[3-fluoro-4-(4-hydroxypiperidin-1-yl)phenyl]urea
1-(4-Chlorophenyl)-3-[3-fluoro-4-(3-methylpiperidin-1-yl)phenyl]urea
1-Benzo[1,3]dioxol-5-yl-3-(3-fluoro-4-piperidin-1-ylphenyl)urea
1-(4-Chlorophenyl)-3-[3-fluoro-4-(2-methylpiperidin-1-yl)phenyl]urea
1-(3-Fluoro-4-piperidin-1-ylphenyl)-3-(4-methoxyphenyl)urea
1-(4-Chloro-2-hydroxyphenyl)-3-(3-fluoro-4-piperidin-1-ylphenyl)urea
1-(3-Fluoro-4-piperidin-1-ylphenyl)-3-[3-(2-methylpyrimidin-4-yl)phenyl]urea
1-(4-Chlorophenyl)-3-[3-fluoro-4-(4-trifluoromethylpiperidin-1-yl)phenyl]urea
1-(4-Chlorophenyl)-3-[3-fluoro-4-(4-methylpiperidin-1-yl)phenyl]urea
1-(3-Fluoro-4-piperidin-1-ylphenyl)-3-(4-phenoxyphenyl)urea
1-(3-Fluoro-4-piperidin-1-ylphenyl)-3-(3-phenoxyphenyl)urea
1-(4-Fluorophenyl)-3-(3-fluoro-4-piperidin-1-ylphenyl)urea
1-(3-Fluoro-4-piperidin-1-ylphenyl)-3-(3-methoxyphenyl)urea
1-(4-Cyanophenyl)-3-(3-fluoro-4-piperidin-1-ylphenyl)urea
1-(4-Chlorophenyl)-3-(4-morpholin-4-ylmethylphenyl)urea
1-(4-Chloro-3-trifluoromethylphenyl)-3-(3-fluoro-4-piperidin-1-ylphenyl)urea
1-(3-Fluoro-4-piperidin-1-ylphenyl)-3-(4-trifluoromethylphenyl)urea
1-(3-Chlorophenyl)-3-(3-fluoro-4-piperidin-1-ylphenyl)urea
1-(4-Chlorophenyl)-3-(3,4,5,6-tetrahydro-2H-[1,2′]bipyridinyl-5′-yl)urea
1-(4-Chlorophenyl)-3-(3-dimethylaminophenyl)urea
1-(4-Chlorophenyl)-3-(3-fluoro-4-morpholin-4-ylphenyl)urea
1-[2-(4-Chlorophenyl)ethyl]-3-(3-pyrrol-1-ylphenyl)urea
1-(3,5-Dichlorophenyl)-3-(3,4,5,6-tetrahydro-2H-[1,2′]bipyridinyl-5′-yl)urea
1-(3-Chlorophenyl)-3-(3,4,5,6-tetrahydro-2H-[1,2′]bipyridinyl-5′-yl)urea
1-(3,5-Bis-trifluoromethylphenyl)-3-(3,4,5,6-tetrahydro-2H-[1,2]bipyridinyl-5′-yl)urea
1-(4-Acetylphenyl)-3-[4-(morpholine-4-carbonyl)phenyl]urea
1-(4-Acetylphenyl)-3-[3-(6-methoxypyridin-2-yl)phenyl]urea
1-(4-Acetylphenyl)-3-(4-morpholin-4-ylmethylphenyl)urea
1-(4-Chlorophenyl)-3-(3-fluoro-4-piperidin-1-ylphenyl)urea
1-(3-Chloro-4-morpholin-4-ylphenyl)-3-(4-chlorophenyl)urea
1-(4-Chlorophenyl)-3-(4-piperidin-1-ylphenyl)urea
1-(4-Acetylphenyl)-3-(3,4,5,6-tetrahydro-2H-[1,2]bipyridinyl-5′-yl)urea
1-(4-Butyrylphenyl)-3-(4-piperidin-1-ylphenyl)urea
1-[2-(4-Chlorophenyl)ethyl]-3-(4-morpholin-4-ylmethylphenyl)urea
1-(4-Chlorophenyl)-3-(1-methyl-1H-indazol-5-yl)urea
1-(4-Chlorophenyl)-3-[3-(2-pyrrolidin-1-ylpyrimidin-4-yl)phenyl]urea
1-(4-Chlorophenyl)-3-(4-pyrazol-1-ylphenyl)urea
1-[2-(4-Chlorophenyl)ethyl]-3-[4-(morpholine-4-carbonyl)phenyl]urea
and pharmaceutically acceptable salts thereof.

33. A pharmaceutical composition comprising a compound of formula (I) as defined in claim 18, or a pharmaceutically acceptable salt thereof, in combination with a pharmaceutically acceptable carrier.

34. A process for the production of a compound of formula (I) as defined in claim 18 which comprises:
a) combining an amine of formula (II) with an isocyanate of formula (III) in a suitable solvent: 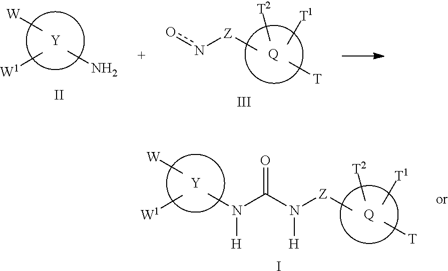
b) combining an amine of formula (IV) with an isocyanate of formula (V) in a suitable solvent: [chemical structure]
